(19) United States
(12) Patent Application Publication
(10) Pub. No.: US 2023/0407320 A1
(43) Pub. Date: Dec. 21, 2023

(54) METHODS AND COMPOSITIONS FOR GENOME MODIFICATION

(71) Applicant: PIONEER HI-BRED INTERNATIONAL, INC., JOHNSTON, IA (US)

(72) Inventors: MAREN L. ARLING, DES MOINES, IA (US); WILLIAM JAMES GORDON-KAMM, URBANDALE, IA (US); JOSHUA K. YOUNG, JOHNSTON, IA (US)

(73) Assignee: PIONEER HI-BRED INTERNATIONAL, INC., JOHNSTON, IA (US)

(21) Appl. No.: 18/250,469

(22) PCT Filed: Oct. 27, 2021
(86) PCT No.: PCT/US21/72049

§ 371 Date: Apr. 25, 2023

Related U.S. Application Data
(60) Provisional application No. 63/107,363, filed on Oct. 29, 2020.

(51) Int. Cl. C12N 15/82; C12N 9/22

(52) U.S. Cl. CPC C12N 15/8205; C12N 2310/20; C12N 9/22

(57) ABSTRACT

Methods and compositions are provided for the transient expression and activity of site-specific DNA modifying agent, morphogenic factors or developmental genes, either alone or in combination for eukaryotic cells. The morphogenic factor and DNA modifying agent may be provided to the same cell or to a different cell than that which was originally transformed. The morphogenic factor, or the double-strand-break-inducing agent, may further comprise a cell penetrating peptide. Exogenously provided DNA modifying agents and/or morphogenic factors need not be segregated away in future generations due to transient activities in the desired cell.

### FIELD

The disclosure relates to the field of molecular biology compositions and methods for modifying a polynucleotide sequence, including the genome of a cell.

### BACKGROUND

Recombinant DNA technology has made it possible to insert DNA sequences at targeted genomic locations and/or modify specific endogenous chromosomal sequences. Site-specific integration techniques, which employ site-specific recombination systems, as well as other types of recombination technologies, have been used to generate targeted insertions of genes of interest in a variety of organism. Genome-editing techniques such as designer zinc finger nucleases (ZFNs), transcription activator-like effector nucleases (TALENs), or homing meganucleases, are available for producing targeted genome perturbations, but these systems employ designed nucleases that need to be redesigned for each target site, which renders them costly and time-consuming to prepare.

Newer technologies utilizing archaeal or bacterial adaptive immunity systems have been identified, called CRISPR (Clustered Regularly Interspaced Short Palindromic Repeats), which comprise different domains of effector proteins that encompass a variety of activities (DNA recognition, binding, and optionally cleavage).

Irrespective of the method used for CRISPR-based genome editing, undesired sequences (for example, helper genes, the endonuclease gene) can be integrated into the genome, requiring later removal through deletion or genetic segregation. There is a need for improved methods of genome editing and component delivery to cells. This is especially true in crops which have a long reproductive timeline, clonally propagated crops, crops which are recalcitrant to transformation, hybrids, or crops that have high genetic variation between generations. One of the benefits of methods described herein is that a genome can be edited. e.g., by introducing a new gene at a targeted locus (SDN3) without integration of a DNA modifying agent.

### SUMMARY

A method of modifying a target site in the genome of a plant cell, the method includes providing to the cell two distinct Rhizobiales bacteria, wherein the first of the two bacteria comprises a first vector, wherein the first vector comprises a heterologous polynucleotide, wherein the heterologous polynucleotide is flanked by polynucleotides comprising homology to a nucleotide sequence at the target site, wherein the second of the two bacteria comprises a second vector, wherein the second vector comprises a polynucleotide encoding a Cas endonuclease, a guide RNA, at least one morphogenic factor, and an anti-regeneration factor; wherein the ratio of the amounts of the first vector and the second vector or the ratio of the amount of the two distinct Rhizobiales bacteria is approximately 1:1 to about 10:1, and wherein the Cas endonuclease creates a double-strand break at or near the target site of the plant cell but the polynucleotide encoding the Cas endonuclease does not integrate into the genome of the same plant cell.

A method of modifying a target site in the genome of a plant cell, the method includes providing to the cell at least two distinct Rhizobiales bacteria, wherein the first of the two bacteria comprises a first vector, wherein the first vector comprises a gene encoding a guide RNA and a heterologous polynucleotide, wherein the heterologous polynucleotide is flanked by polynucleotides comprising homology to a nucleotide sequence at the target site, wherein the second of the two bacteria comprises a second vector, wherein the second vector comprises one or more polynucleotides encoding a Cas endonuclease, at least one morphogenic factor, and a gene conferring anti-regeneration properties; wherein the ratio of the amounts of the first vector and the second vector or the ratio of the amount of the two distinct Rhizobiales bacteria is approximately 1:1 to about 10:1, and wherein the Cas endonuclease creates a double-strand break at or near the target site of the genome of the plant cell but the T-DNA comprising the Cas endonuclease does not integrate into the genome of the same plant cell.

A method of modifying a target site in the genome of a plant cell, the method includes providing to the cell one Rhizobiales strain comprising first and second binary plasmids, wherein each of the two binary plasmids comprise distinct T-DNA sequences, wherein the first binary plasmid comprises a low-copy ORI, and a T-DNA encoding a Cas endonuclease and a morphogenic factor, wherein the second binary plasmid comprises a higher copy ORI and comprises a T-DNA encoding a heterologous polynucleotide, wherein the ratio of the amounts of the first plasmid and the second plasmid is approximately 1:1 to about 1:10, and wherein the Cas endonuclease creates a double-strand break at or near the target site of the plant cell but does not integrate into the genome of the same plant cell.

In certain embodiment, the heterologous polynucleotide is integrated through site-directed homologous recombination. In certain embodiment, the plant cell is a monocot cell or a dicot cell. In certain embodiment, the morphogenic factor is Wuschel. In certain embodiment, the Cas endonuclease comprises fewer than about 1500, 1200, 100, 800 and 500 amino acids. In certain embodiment, the heterologous polynucleotide is a template for homology-directed repair of a double-strand break at the target site. In certain embodiment, the at least one of the Rhizobiales is Agrobacterium. In certain embodiment, the heterologous polynucleotide is a donor polynucleotide for integration at the target site.

A synthetic composition comprising two distinct Rhizobiales bacteria, wherein the first of the two bacteria comprises a first vector, wherein the first vector comprises a heterologous polynucleotide, wherein the heterologous polynucleotide is flanked by polynucleotides comprising homology to a nucleotide sequence at the target site, wherein the second of the two bacteria comprises a second vector, wherein the second vector comprises one or more polynucleotides encoding a Cas endonuclease, a guide RNA, and at least one morphogenic factor; wherein the ratio of the amounts of the first vector and the second vector is approximately 1:1 to about 10:1.

A synthetic composition comprising two different Rhizobiales bacteria, wherein the first of the two bacteria comprises a first vector, wherein the first vector comprises a gene encoding a guide RNA and a heterologous polynucleotide, wherein the heterologous polynucleotide is flanked by polynucleotides comprising homology to a nucleotide sequence at the target site, wherein the second of the two bacteria comprises a second vector, wherein the second vector comprises one or more polynucleotides encoding a Cas endonuclease and at least one morphogenic factor; wherein the ratio of the amounts of the first vector and the second vector is approximately 1:1 to about 10:1.

A synthetic composition comprising one Rhizobiales strain comprising first and second plasmids, wherein each of the two plasmids comprise one or more distinct T-DNA sequences, wherein the first plasmid comprises a low-copy ORI and comprises a T-DNA encoding a Cas endonuclease and a morphogenic factor, wherein the second plasmid comprises a higher copy ORI and comprises a T-DNA encoding a heterologous polynucleotide, wherein the ratio of the amounts of the first plasmid and the second plasmid is approximately 1:1 to about 1:10. In certain embodiments, the synthetic composition is a plant cell. In certain embodiments, the plant cell is a monocot cell or a dicot cell. In certain embodiment, the at least one of the Rhizobiales is Agrobacterium. In certain embodiment, the Cas endonuclease comprises fewer than about 1500, 1200, 100, 800 and 500 amino acids. In certain embodiments, the synthetic composition further includes an anti-regeneration factor. In certain embodiments, the plant cell comprising in its genome the polynucleotide encoding the Cas endonuclease is not selected due to the presence or activity of the anti-regeneration factor. In certain embodiments, the Cas endonuclease is a Type II or Type V CRISPR-Cas endonuclease.

In certain embodiments, the plant cell is a haploid plant cell. In certain embodiments, the plant cell is a haploid plant cell and the DNA modification happens before or during chromosome doubling stage. In certain embodiments, the at least one of the Cas endonuclease, the morphogenic factor, and the anti-regeneration factor the plant cell comprises a heterologous cell penetrating peptide (CPP) or an intracellular transfusion domain.

A method of modifying a target site in the genome of a plant cell through endogenous activation of one or more genes, the method comprising providing to the cell (a) a first polynucleotide encoding a deactivated CRISPR-Cas polypeptide (dCas) that is capable of site-specifically binding to an endogenous target site comprising a polynucleotide involved in cellular regeneration, but incapable of introducing a double-strand break at the endogenous target site, wherein the dCas is operably linked to a transcriptional activator, the transcriptional activator capable of increasing the expression of the polynucleotide involved in cellular regeneration; (b) providing a second polynucleotide comprising a heterologous polynucleotide flanked by polynucleotides comprising homology to a nucleotide sequence at the target site to be modified; (c) providing a third polynucleotide encoding a Cas endonuclease, a guide RNA, and an anti-regeneration factor; wherein the ratio of the amounts of the polynucleotide comprising the Cas endonuclease and the polynucleotide comprising the heterologous polynucleotide is approximately 1:1 to about 1:10, and wherein the Cas endonuclease creates a double-strand break at or near the target site of the plant cell but the polynucleotide encoding the Cas endonuclease does not integrate into the genome of the same plant cell.

In certain embodiments, the dCas is fused to a transcriptional activation domain capable of initiating transcription from an endogenous morphogenic gene is WUSCHEL, BBM or a combination thereof. In certain embodiments, the polynucleotides are provided through one or more strains of Rhizobiales. In certain embodiments, the polynucleotides are provided through particle bombardment. In certain embodiments, the polynucleotides are present in one or more T-DNAs in the same Agrobacterium strain or two or more distinct Agrobacteria strains. In certain embodiments, the dCas polypeptide is a Type II or a Type V CRISPR-Cas polypeptide.

A synthetic composition comprising (a) a first polynucleotide encoding a deactivated CRISPR-Cas polypeptide (dCas) that is capable of site-specifically binding to an endogenous target site comprising a polynucleotide involved in cellular regeneration, but incapable of introducing a double-strand break at the endogenous target site, wherein the dCas is operably linked to a transcriptional activator, the transcriptional activator capable of increasing the expression of the polynucleotide involved in cellular regeneration; (b) providing a second polynucleotide comprising a heterologous polynucleotide flanked by polynucleotides comprising homology to a nucleotide sequence at the target site to be modified; (c) providing a third polynucleotide encoding a Cas endonuclease, a guide RNA, and an anti-regeneration factor. In certain embodiments, the one or more of the dCas is fused to a cell penetrating peptide.

A method of regeneration of a genome modified plant, the method comprising providing one or more polynucleotides that encode a site-specific DNA modifying agent and optionally, a morphogenic factor to a plant cell, wherein the DNA modifying agent and optionally, the morphogenic factor diffuses or is transported into one or more adjacent plant cells; modifying the genome of the one or more adjacent plant cells; regenerating the one or more adjacent plant cells into one or more genome modified plants, wherein the one or more genome modified plants do not comprise the polynucleotide sequence encoding the DNA modifying agent and optionally the morphogenic factor in the absence of a separate segregation step by crossing with another plant. In certain embodiments the DNA modifying element, the morphogenic element and other exogenously introduced DNA sequences need not be removed (e.g., through targeted deletions, or through genetic segregation through back-crossing or other breeding methods) due to the altruistic nature of editing nearby or neighboring cells. These methods help reduce the amount of time required to develop a genome edited line for product development by reducing the need for genetic segregations.

In certain embodiments, the DNA modifying agent is a CRISPR-Cas polypeptide that is capable of moving from one cell to another as a polypeptide or in the form of an RNA sequence capable of being translated into a polypeptide. In certain embodiments, the DNA modifying agent is a CRISPR-Cas polypeptide and is operably linked to a cell penetrating peptide. In certain embodiments, the optional morphogenic factor is operably linked to a cell penetrating peptide. In certain embodiments, the cell that receives the polynucleotide encoding the DNA modifying agent is not selected, but the genome modified cell is regenerated into a genome modified plant. In certain embodiments, the DNA modifying agent is a CRISPR-Cas polypeptide and the guide RNA is encoded by the same polynucleotide that encodes the CRISPR-Cas polypeptide. In certain embodiments, the DNA modifying agent is a base editor.

In certain embodiments, the optional morphogenic factor is encoded by an expression construct that is distinct from the polynucleotide that encodes the DNA modifying agent. In certain embodiments, the polynucleotide that encodes the DNA modifying agent, a guide RNA and the optional morphogenic factor are present in one or more distinct expression cassettes. In certain embodiments, the DNA modifying agent is a CRISPR-Cas deactivated polypeptide (dCas). In certain embodiments, the methods or the compositions disclosed herein further include a heterologous donor polynucleotide.

In certain embodiments, polynucleotide that encodes the DNA modifying agent and the optional morphogenic factor are present in the plant cell at a ratio of about 1:1 to about 1:5, 1:8, 1:10 and to about 1:100.

In certain embodiments, a heterologous polynucleotide is initially integrated into the nucleus where it is either transiently expressed, then degraded, or stably integrated at a specific locus. In some embodiments, a first viable plant containing a genomic DNA is provided that contains a donor DNA flanked by a plurality of recognition sequences and the plant genomic target locus, wherein the plant genomic target locus also contains at least one recognition sequence. In some embodiments, a second viable plant containing a site-specific nuclease is provided. In some embodiments, the first and second viable plants are crossed to produce F1 seed. In some embodiments, the site-specific nuclease is expressed and cleaves at least one site specific nuclease recognition sequence to release a donor polynucleotide and to create a double strand break within the plant genomic locus. In some embodiments, the donor DNA is integrated within the plant genomic locus. In some embodiments, the donor DNA is integrated within the plant genomic locus via a non-homologous end joining mechanism. In some embodiments, the donor DNA is integrated within the plant genomic locus via homology-directed repair.

### BRIEF DESCRIPTION OF THE DRAWINGS

The disclosure can be more fully understood from the following detailed description and the accompanying drawings, which form a part of this application.

### FIGURES

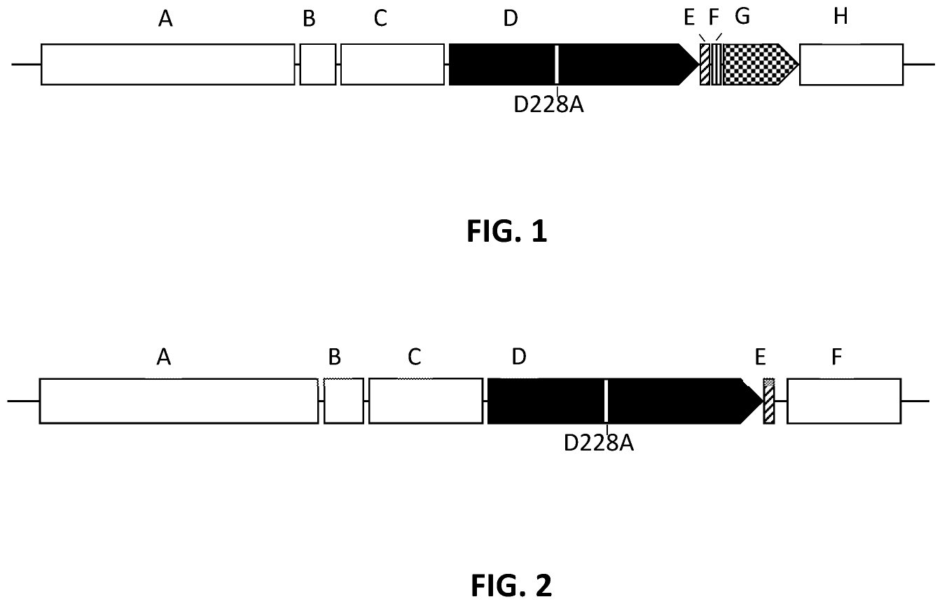

FIG. 1 is a schematic illustration of a transformation cassette used. The following references related to the identified parts of the figure. A: Zea mays UBI promoter; B: Zea mays UBI 5′ UTR; C: Zea mays UBI Intron 1; D: Zea mays optimized A40G+E81G+A87K+D228A+T335R nuclease inactive or dead (d) cas-alpha 10 gene including ST-LS1 Intron 2. A40G+E81G+A87K+D228A+T335R is comprised of alterations that enhance the cellular activity of Cas-alpha 10, A40G+E81G+A87K+T335R, plus an additional change that abolishes target cleavage, D228A; E: Zea mays optimized sequence encoding SV40 NLS; F: Zea mays optimized sequence encoding a glycine, arginine and alanine linker; G: Zea mays optimized sequence encoding the carboxyl terminal acidic transcriptional activation domain of the cold binding factor 1 (CBF1) protein from Arabidopsis thaliana; and H: Zea mays UBI terminator.

FIG. 2 is a schematic illustration of a cassette used in the methods disclosed herein. The following references related to the identified parts of the figure. A: Zea mays UBI promoter; B: Zea mays UBI 5′ UTR; C: Zea mays UBI Intron 1; D: Zea mays optimized A40G+E81G+A87K+D228A+T335R nuclease inactive or dead (d) cas-alpha 10 gene including ST-LS1 Intron 2. A40G+E81G+A87K+D228A+T335R is comprised of alterations that enhance the cellular activity of Cas-alpha 10, A40G+E81G+A87K+T335R, plus an additional change that abolishes target cleavage, D228A; E: Zea mays optimized sequence encoding SV40 NLS and F: Zea mays UBI terminator.

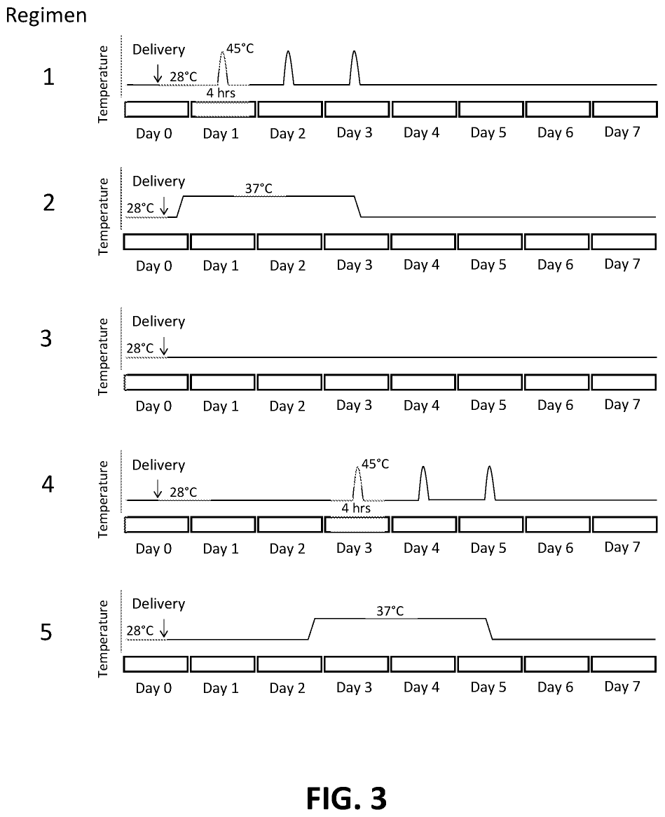

FIG. 3 shows five different transformation regimens that were tested for delivery of constructs herein. Each regimen 1-5 indicates heat treatment duration, number of heat treatments, and when (days after construct delivery) heat treatment (if any) was administered.

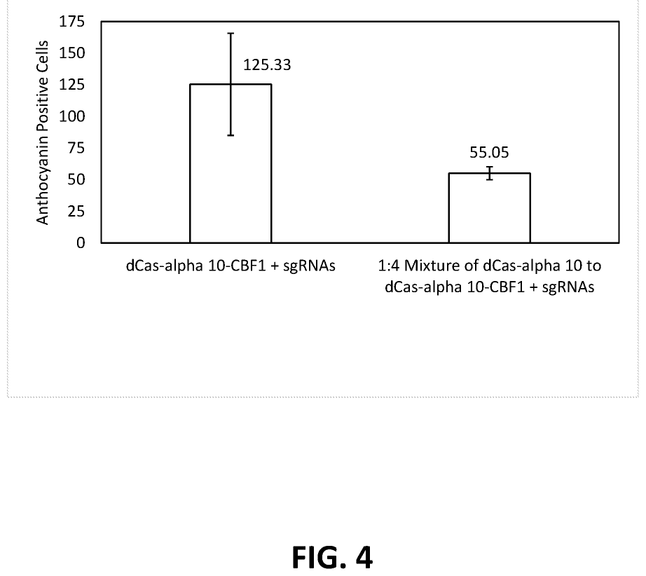

FIG. 4 shows the average number of anthocyanin positive cells expressing dCas-alpha 10-CBF1 resulting in a dimer comprised solely of dCas-alpha 10-CBF1 monomers as compared to a mixture (heterodimers) of dCas-alpha 10 and dCas-alpha 10-CBF1, each with respective sgRNAs.

### REFERENCE TO SEQUENCE LISTING SUBMITTED ELECTRONICALLY

The official copy of the sequence listing is submitted electronically via EFS-Web as an ASCII formatted sequence listing with a file named 8189-WO-PCT_ST25.txt created on Oct. 26, 2021 and having a size of 648 kilobytes and is filed concurrently with the specification. The sequence listing comprised in this ASCII formatted document is part of the specification and is herein incorporated by reference in its entirety.

### DETAILED DESCRIPTION

Methods and compositions are provided for the transient expression and activity of site-specific DNA modifying agent, morphogenic factors or developmental genes, either alone or in combination for eukaryotic cells. The morphogenic factor and DNA modifying agent may be provided to the same cell or to a different cell than that which was originally transformed. The morphogenic factor, or the double-strand-break-inducing agent, may further comprise a cell penetrating peptide. Stable integration of DNA is inhibited through the use of a low constitutive promoter driving an anti-regeneration gene. Transient expression of the anti-regeneration gene does not kill the cell, but stable integration inhibits regeneration, or is lethal. Transient expression of and activity of both double-strand-break-inducing agent and morphogenic factors are sufficient to provide editing for SNP conferring resistance, SDN2, or SDN3 of a selectable marker and trait of interest.

Compositions are provided for Cas endonuclease-mediated genome modification. The present disclosure further includes compositions and methods for genome modification of a target sequence in the genome of a cell, for gene editing, and for inserting a polynucleotide of interest into the genome of a cell.

In certain embodiments, the amount of the various components (DNA modifying agent: morphogenic factor; or donor polynucleotide: morphogenic factor; or Cas enzyme/gRNA: morphogenic factor; or donor polynucleotide and Cas enzyme: morphogenic factor; or further combinations thereof) described herein can range in a ratio of about 1:1; 2:1; 4:1; 9:1; 10:1; 20:1; 50:1; 100:1 to about 1:1, 1:2, 1:5 and to about 1:10. These ratio can be adapted to a variety of factors that influence the amount of DNA delivered into a cell. These include for example, copy numbers of plasmids, Agrobacterium concentration, viral DNA amount if viral infection is the mode of delivery, origin of replication differences, Rhizobiales strain differences, concentration of DNA on particles for particle bombardment, and any other form of delivery where the amount or concentration of the components delivered can be controlled, measured or otherwise modulated.

Disclosed herein are improved Agrobacterium-mediated genome editing solutions for target cell genomes, wherein helper genes (such as morphogenic factors, e.g., Wuschel) and the Cas endonuclease are provided by separate Agrobacteria and T-DNAs, to provide such components without their integration into the target cell's genome. Other Rhizobiales-mediated transformation, such as the use of Ochrobacterium, is further contemplated.

In a transformation with two different Agrobacteria, at some frequency there will be infection of both into a single cell. While first 1st donor T-strand does integrate stably, the second is able to transiently express genes, but not integrate into the genome to produce a viable plant. This method filters out/selects against any events where the second helper T-strand stably integrates. This method is useful for any plant, for example any crop plant, or in plants where segregating unwanted loci out of the germplasm is difficult (e.g., sugarcane, tree crops, clonally propagated crops, crops with perfect flowers, etc.) This novel method eliminates the need for segregation of unwanted DNA, leaving only the modified locus. In some aspects, the method relies on allowing transformation using a mixture of two Rhizobiales strains (comprising two different T-DNA sequences) to drive homologous recombination editing or base-pair editing with no integration of any accessory component, such as a morphogenic factor or the Cas endonuclease genes. By adjusting the ratio of the two Rhizobiales strains, targeted genomic modifications can be recovered with no random integration of any accessory component. In some aspects, the ratio of the two Rhizobiales (WUS and/or Cas9 Agro to HDR template Agro) is 9:1, 1:1, 1:9, 1:99. In some aspects, the Rhizobiales strain is Agrobacterium tumefaciens.

In certain embodiments, the T-DNA delivery can be influenced by mixing Agrobacteria (or for example different strains of Rhizobiales) at various ratios where the two Agrobacteria contain a plasmid with the same origin of replication but with two different T-DNAs (e.g., T-DNA 1 and T-DNA2). This ratio can extend from 1:1 up to 1:100 where either Agrobacterium can be present at the higher ratio.

In certain embodiments, T-DNA delivery can also be influenced by using two T-DNA containing plasmids in the same Agrobacterium cell, where different origins of replication impact the relative copy numbers of the plasmids within the same Agro. Different origins of replication have been demonstrated to result in low to high copy number (e.g., Zhi et al. 2015, Plant cell Reports 34, 745-754). For example, within the AGL0 strain, a vector containing a RK2 ORI had a relative copy number of 1 plasmid copy per cell. Whereas, a low copy number repABC-containing vector had a relative copy number of 0.22 plasmid copies per cell. Likewise, a AGL0 containing a high copy ORI pVS1 had a relative copy number of 2.65 plasmid copies per cell.

T-DNA delivery can also be influenced by using two agrobacteria, each containing its own T-DNA plasmid, where one agrobacterium contains a low copy number origin of replication and the other contains a high copy number origin of replication.

T-DNA delivery can also be influenced where one plasmid has reduced TDNA delivery due to a RB sequence which does not match the corresponding VIRD2 strain of origin. The second plasmid has a T-DNA where the RB sequence and VIRD2 come from the same strain of origin, and thus will have higher T-DNA delivery.

Double-strand breaks induced by double-strand-break-inducing agents, such as endonucleases that cleave the phosphodiester bond within a polynucleotide chain, can result in the induction of DNA repair mechanisms, including the non-homologous end-joining pathway, and homologous recombination. Endonucleases include a range of different enzymes, including restriction endonucleases (see e.g., Roberts et al., (2003) Nucleic Acids Res 1:418-20), Roberts et al., (2003) Nucleic Acids Res 31:1805-12, and Belfort et al., (2002) in Mobile DNA II, pp. 761-783, Eds. Craigie et al., (ASM Press, Washington, DC)), meganucleases (see e.g., WO 2009/114321; Gao et al. (2010) Plant Journal 1:176-187), TAL effector nucleases or TALENs (see e.g., US20110145940, Christian, M., T. Cermak, et al. 2010. Targeting DNA double-strand breaks with TAL effector nucleases. Genetics 186(2): 757-61 and Boch et al., (2009), Science 326(5959): 1509-12), Zinc Finger Nucleases (see e.g., Kim, Y. G., J. Cha, et al. (1996). “Hybrid restriction enzymes: zinc finger fusions to FokI cleavage”), and CRISPR-Cas endonucleases (see e.g., WO2007/025097 application published Mar. 1, 2007).

Once a double-strand break is induced in the genome, cellular DNA repair mechanisms are activated to repair the break. There are two DNA repair pathways. One is termed nonhomologous end-joining (NHEJ) pathway (Bleuyard et al., (2006) DNA Repair 5:1-12) and the other is homology-directed repair (HDR). The structural integrity of chromosomes is typically preserved by NHEJ, but deletions, insertions, or other rearrangements (such as chromosomal translocations) are possible (Siebert and Puchta, 2002, Plant Cell 14:1121-31; Pacher et al., 2007, Genetics 175:21-9. The HDR pathway is another cellular mechanism to repair double-stranded DNA breaks, and includes homologous recombination (HR) and single-strand annealing (SSA) (Lieber. 2010 Annu. Rev. Biochem. 79:181-211).

Terms used in the claims and specification are defined as set forth below unless otherwise specified. It must be noted that, as used in the specification and the appended claims, the singular forms “a,” “an” and “the” include plural referents unless the context clearly dictates otherwise.

“Genome” as it applies to a prokaryotic and eukaryotic cell or organism cells encompasses not only chromosomal DNA found within the nucleus, but organelle DNA found within subcellular components (e.g., mitochondria, or plastid) of the cell.

“Target site”, “target sequence”, “target site sequence, “target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, a locus, or any other DNA molecule in the genome (including chromosomal, chloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature. terms “endogenous target sequence” and “native target sequence” are used interchangeable herein to refer to a target sequence that is endogenous or native to the genome of a cell and is at the endogenous or native position of that target sequence in the genome of the cell. “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

“Polynucleotide” is not intended to limit the present disclosure to polynucleotides comprising DNA or RNA. Such deoxyribonucleotides and ribonucleotides include both naturally occurring molecules and synthetic analogues. The polynucleotides disclosed herein also encompass all forms of sequences including, but not limited to, single-stranded forms, double-stranded forms, hairpins, stem-and-loop structures, and the like.

“Isolated” nucleic acid molecule (or DNA) is used herein to refer to a nucleic acid sequence (or DNA) that is no longer in its natural environment, for example in an in vitro or in a heterologous recombinant bacterial or plant host cell. An isolated nucleic acid molecule, or biologically active portion thereof, is substantially free of other cellular material or culture medium when produced by recombinant techniques, or substantially free of chemical precursors or other chemicals when chemically synthesized. An isolated nucleic acid is free of sequences (optimally protein encoding sequences) that naturally flank the nucleic acid (i.e., sequences located at the 5′ and 3′ ends of the nucleic acid) in the genomic DNA of the organism from which the nucleic acid is derived.

“Variants” is intended to mean substantially similar sequences. For polynucleotides, a variant comprises a polynucleotide having a deletion (i.e., truncations) at the 5′ and/or 3′ end and/or a deletion and/or addition of one or more nucleotides at one or more internal sites within the native polynucleotide and/or a substitution of one or more nucleotides at one or more sites in the native polynucleotide.

“Functional variant”, “variant that is functionally equivalent” and “functionally equivalent variant” of a protein, including any described herein, are used interchangeably herein, and refer to a variant of the protein in which the protein's native ability is retained either partially or completely. In some aspects, the function may be entirely new.

“Fragment” is intended a portion of the polynucleotide or a portion of the amino acid sequence and hence protein encoded thereby. Fragments of a polynucleotide may encode protein fragments that retain the biological activity of the native protein and hence influence male fertility. Alternatively, fragments of a polynucleotide that are useful as hybridization probes generally do not encode fragment proteins retaining biological activity. Thus, fragments of a nucleotide sequence may range from at least about 20 nucleotides, about 50 nucleotides, about 100 nucleotides, and up to the full-length polynucleotide encoding the polypeptides disclosed herein.

“Functional fragment,” “fragment that is functionally equivalent,” and “functionally equivalent fragment” are used interchangeably herein. These terms refer to a portion or subsequence of a polypeptide sequence of the present disclosure in which its native ability is retained.

“Heterologous” in reference to a sequence is a sequence that originates from a foreign species or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

“Percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100).

“Introducing” is intended to mean presenting to the host cell, plant, plant cell or plant part the polynucleotide or polypeptide in such a manner that the sequence gains access to the interior of a cell of the plant or organism.

“Crossed” or “cross” refers to a sexual cross and involved the fusion of two haploid gametes via pollination to produce diploid progeny (e.g., cells, seeds or plants). encompasses both the pollination of one plant by another and selfing (or self-pollination, e.g., when the pollen and ovule are from the same plant).

“Closely linked”, in the present application, means that recombination between two linked loci occurs with a frequency of equal to or less than about 10% (i.e., are separated on a genetic map by not more than 10 cM). Put another way, the closely linked loci co-segregate at least 90% of the time. Marker loci are especially useful with respect to the subject matter of the current disclosure when they demonstrate a significant probability of co-segregation (linkage) with a desired trait (e.g., resistance to southern corn rust). Closely linked loci such as a marker locus and a second locus can display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination a frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “proximal to” each other. In some cases, two different markers can have the same genetic map coordinates. In that case, the two markers are in such close proximity to each other that recombination occurs between them with such low frequency that it is undetectable.

“Line” or “strain” is a group of individuals of identical parentage that are generally inbred to some degree and that are generally homozygous and homogeneous at most loci (isogenic or near isogenic). “subline” refers to an inbred subset of descendents that are genetically distinct from other similarly inbred subsets descended from the same progenitor.

“Elite line” is any line that has resulted from breeding and selection for superior agronomic performance.

“Enhanced trait” as used in describing the aspects of the present disclosure includes improved or enhanced water use efficiency or drought tolerance, osmotic stress tolerance, high salinity stress tolerance, heat stress tolerance, enhanced cold tolerance, including cold germination tolerance, increased yield, enhanced nitrogen use efficiency, early plant growth and development, late plant growth and development, enhanced seed protein, and enhanced seed oil production

“Genetic markers” are nucleic acids that are polymorphic in a population and where the alleles of which can be detected and distinguished by one or more analytic methods, e.g., RFLP, AFLP, isozyme, SNP, SSR, and the like. also refers to nucleic acid sequences complementary to the genomic sequences, such as nucleic acids used as probes. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also known for the detection of expressed sequence tags (ESTs) and SSR markers derived from EST sequences and randomly amplified polymorphic DNA (RAPD).

“Genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

“Germplasm” refers to genetic material of or from an individual (e.g., a plant), a group of individuals (e.g., a plant line, variety or family), or a clone derived from a line, variety, species, or culture, or more generally, all individuals within a species or for several species (e.g., maize germplasm collection or Andean germplasm collection). The germplasm can be part of an organism or cell, or can be separate from the organism or cell. In general, germplasm provides genetic material with a specific molecular makeup that provides a physical foundation for some or all of the hereditary qualities of an organism or cell culture. germplasm includes cells, seed or tissues from which new plants may be grown, or plant parts, such as leafs, stems, pollen, or cells, that can be cultured into a whole plant.

“Grain” is intended the mature seed produced by commercial growers for purposes other than growing or reproducing the species. Progeny, variants, and mutants of the regenerated plants are also included within the scope of the disclosure, provided that these parts comprise the introduced nucleic acid sequences

“Haplotype” is the genotype of an individual at a plurality of genetic loci, i.e. a combination of alleles. Typically, the genetic loci described by a haplotype are physically and genetically linked, i.e., on the same chromosome segment.

“Heterogeneity” is used to indicate that individuals within the group differ in genotype at one or more specific loci.

“Heterosis”, or The heterotic response of material, can be defined by performance which exceeds the average of the parents (or high parent) when crossed to other dissimilar or unrelated groups.

“Heterotic group” comprises a set of genotypes that perform well when crossed with genotypes from a different heterotic group (Hallauer et al. (1998) Corn breeding, p. 463-564. In G. F. Sprague and J. W. Dudley (ed.) Corn and corn improvement). Inbred lines are classified into heterotic groups, and are further subdivided into families within a heterotic group, based on several criteria such as pedigree, molecular marker-based associations, and performance in hybrid combinations (Smith et al. (1990) Theor. Appl. Gen. 80:833-840). The two most widely used heterotic groups in the United States are referred to as “Iowa Stiff Stalk Synthetic” (also referred to herein as “stiff stalk”) and “Lancaster” or “Lancaster Sure Crop” (sometimes referred to as NSS, or non-Stiff Stalk).

“Hybrid” refers to the progeny obtained between the crossing of at least two genetically dissimilar parents.

“Inbred” refers to a line that has been bred for genetic homogeneity.

“Introgression” refers to the transmission of a desired allele of a genetic locus from one genetic background to another. For example, introgression of a desired allele at a specified locus can be transmitted to at least one progeny plant via a sexual cross between two parent plants, where at least one of the parent plants has the desired allele within its genome. Alternatively, for example, transmission of an allele can occur by recombination between two donor genomes, e.g., in a fused protoplast, where at least one of the donor protoplasts has the desired allele in its genome. The desired allele can be, e.g., a transgene, a modified (mutated or edited) native allele, or a selected allele of a marker or QTL.

“Linkage disequilibrium” is most commonly assessed using the measure r2, which is calculated using the formula described by Hill, W. G. and Robertson, A, Theor. Appl. Genet. 38:226-231(1968). When r2=1, complete LD exists between the two marker loci, meaning that the markers have not been separated by recombination and have the same allele frequency. The r2 value will be dependent on the population used. Values for r2 above ⅓ indicate sufficiently strong LD to be useful for mapping (Ardlie et al., Nature Reviews Genetics 3:299-309 (2002)). Hence, alleles are in linkage disequilibrium when r2 values between pairwise marker loci are greater than or equal to 0.33, 0.4, 0.5, 0.6, 0.7, 0.8, 0.9, or 1.0. Linkage disequilibrium refers to a non-random segregation of genetic loci or traits (or both). In either case, linkage disequilibrium implies that the relevant loci are within sufficient physical proximity along a length of a chromosome so that they segregate together with greater than random (i.e., non-random) frequency. Markers that show linkage disequilibrium are considered linked. Linked loci co-segregate more than 50% of the time, e.g., from about 51% to about 100% of the time. In other words, two markers that co-segregate have a recombination frequency of less than 50% (and by definition, are separated by less than 50 cM on the same linkage group.) linkage can be between two markers, or alternatively between a marker and a locus affecting a phenotype. A marker locus can be “associated with” (linked to) a trait. The degree of linkage of a marker locus and a locus affecting a phenotypic trait is measured, e.g., as a statistical probability of co-segregation of that molecular marker with the phenotype (e.g., an F statistic or LOD score).

“Linkage” is used to describe the degree with which one marker locus is associated with another marker locus or some other locus. The linkage relationship between a molecular marker and a locus affecting a phenotype is given as “probability” or “adjusted probability”. Linkage can be expressed as a desired limit or range. For example, in some embodiments, any marker is linked (genetically and physically) to any other marker when the markers are separated by less than 50, 40, 30, 25, 20, or 15 map units (or cM) of a single meiosis map (a genetic map based on a population that has undergone one round of meiosis, such as e.g. an F2; the IBM2 maps consist of multiple meiosis). In some aspects, it is advantageous to define a bracketed range of linkage, for example, between 10 and 20 cM, between 10 and 30 cM, or between 10 and 40 cM. The more closely a marker is linked to a second locus, the better an indicator for the second locus that marker becomes. Thus, “closely linked loci” such as a marker locus and a second locus display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “in proximity to” each other. Since one cM is the distance between two markers that show a 1% recombination frequency, any marker is closely linked (genetically and physically) to any other marker that is in close proximity, e.g., at or less than 10 cM distant. Two closely linked markers on the same chromosome can be positioned 9, 8, 7, 6, 5, 4, 3, 2, 1, 0.75, 0.5 or 0.25 cM or less from each other.

“Plant” generically includes whole plants, plant organs, plant tissues, seeds, plant cells, seeds and progeny of the same. The plant is a monocot or dicot. Plant cells include, without limitation, cells from seeds, suspension cultures, embryos, meristematic regions, callus tissue, leaves, roots, shoots, gametophytes, sporophytes, pollen and microspores. A “plant element” is intended to reference either a whole plant or a plant component, which may comprise differentiated and/or undifferentiated tissues, for example but not limited to plant tissues, parts, and cell types. In one embodiment, a plant element is one of the following: whole plant, seedling, meristematic tissue, ground tissue, vascular tissue, dermal tissue, seed, leaf, root, shoot, stem, flower, fruit, stolon, bulb, tuber, corm, keiki, shoot, bud, tumor tissue, and various forms of cells and culture (e.g., single cells, protoplasts, embryos, callus tissue). It should be noted that a protoplast is not technically “intact” plant cell (as naturally found with all components), as protoplasts lack a cell wall. plant organ” refers to plant tissue or a group of tissues that constitute a morphologically and functionally distinct part of a plant. A plant element is synonymous with a portion of a plant, and refers to any part of the plant, and can include distinct tissues and/or organs, and may be used interchangeably with tissue” throughout. Similarly, a plant reproductive element” is intended to generically reference any part of a plant that is able to initiate other plants via either sexual or asexual reproduction of that plant, for example but not limited to: seed, seedling, root, shoot, cutting, scion, graft, rhizome, stolon, bulb, tuber, corm, keiki, or bud. The plant element may be in plant or in a plant organ, tissue culture, or cell culture.

“Plant part” refers to plant cells, plant protoplasts, plant cell tissue cultures from which plants can be regenerated, plant calli, plant clumps, and plant cells that are intact in plants or parts of plants such as embryos, pollen, ovules, egg cells, seeds, leaves, flowers, branches, fruit, kernels, ears, cobs, husks, stalks, roots, root tips, anthers, and the like, as well as the parts themselves. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

“Plant-optimized nucleotide sequence” is a nucleotide sequence that has been optimized for expression in plants, particularly for increased expression in plants. A plant-optimized nucleotide sequence includes a codon-optimized gene. A plant-optimized nucleotide sequence can be synthesized by modifying a nucleotide sequence encoding a protein such as, for example, a Cas endonuclease as disclosed herein, using one or more plant-preferred codons for improved expression. See, for example, Campbell and Gowri (1990) Plant Physiol. 92:1-11 for a discussion of host-preferred codon usage.

“Progeny” comprises any subsequent generation of a plant.

“Target” site or plant or cell means one that is the recipient of an intended introduction of a molecule, or one in which a polynucleotide modification has been affected.

“Stable transformation” is a transformation in which the polynucleotide construct introduced into a plant integrates into the genome of the plant and is capable of being inherited by the progeny thereof.

“Transient transformation” means that a polynucleotide is introduced into the plant and does not integrate into the genome of the plant

The meaning of abbreviations is as follows: “sec” means second(s), “min” means minute(s), “h” means hour(s), “d” means day(s), “uL” means microliter(s), “mL” means milliliter(s), “L” means liter(s), “uM” means micromolar, “mM” means millimolar, “M” means molar, “mmol” means millimole(s), “umole” or “umole” mean micromole(s), “g” means gram(s), “ug” or “ug” means microgram(s), “ng” means nanogram(s), “U” means unit(s), “bp” means base pair(s) and “kb” means kilobase(s).

### Cas Endonucleases

Endonucleases are enzymes that cleave the phosphodiester bond within a polynucleotide chain, and include restriction endonucleases that cleave DNA at specific sites without damaging the bases. Examples of endonucleases include restriction endonucleases, meganucleases, TAL effector nucleases (TALENs), zinc finger nucleases, and Cas (CRISPR-associated) effector endonucleases.

As used herein, the term “Cas gene” refers to a gene that is generally coupled, associated or close to or in the vicinity of flanking CRISPR loci.

“CRISPR” (Clustered Regularly Interspaced Short Palindromic Repeats) loci refers to certain genetic loci encoding components of DNA cleavage systems, for example, used by bacterial and archaeal cells to destroy foreign DNA (Horvath and Barrangou, 2010, Science 327:167-170; WO2007025097, published 1 Mar. 2007). A CRISPR locus can consist of a CRISPR array, comprising short direct repeats (CRISPR repeats) separated by short variable DNA sequences (called spacers), which can be flanked by diverse Cas (CRISPR-associated) genes.

Cas endonucleases, either as single effector proteins or in an effector complex with other components, unwind the DNA duplex at the target sequence and optionally cleave at least one DNA strand, as mediated by recognition of the target sequence by a polynucleotide (such as, but not limited to, a crRNA or guide RNA) that is in complex with the Cas effector protein. Such recognition and cutting of a target sequence by a Cas endonuclease typically occurs if the correct protospacer-adjacent motif (PAM) is located at or adjacent to the 3′ end of the DNA target sequence. Alternatively, a Cas endonuclease herein may lack DNA cleavage or nicking activity, but can still specifically bind to a DNA target sequence when complexed with a suitable RNA component. (See also U.S. Patent Application US20150082478 published 19 Mar. 2015 and US20150059010 published 26 Feb. 2015). Cas endonucleases may occur as individual effectors (Class 2 CRISPR systems) or as part of larger effector complexes (Class I CRISPR systems).

The Cas endonuclease is guided by a single CRISPR RNA (crRNA) through direct RNA-DNA base-pairing to recognize a DNA target site that is in close vicinity to a protospacer adjacent motif (PAM) (Jore, M. M. et al., 2011, Nat. Struct. Mol. Biol. 18:529-536, Westra, E. R. et al., 2012, Molecular Cell 46:595-605, and Sinkunas, T. et al., 2013, EMBO J. 32:385-394).

The guide polynucleotide enables target recognition, binding, and optionally cleavage by the Cas endonuclease, and can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide RNA” or “gRNA” (US20150082478 published 19 Mar. 2015 and US20150059010 published 26 Feb. 2015). A guide polynucleotide may be engineered or synthetic.

The guide polynucleotide includes a chimeric non-naturally occurring guide RNA comprising regions that are not found together in nature (i.e., they are heterologous with each other). For example, a chimeric non-naturally occurring guide RNA comprising a first nucleotide sequence domain (referred to as Variable Targeting domain or VT domain) that can hybridize to a nucleotide sequence in a target DNA, linked to a second nucleotide sequence that can recognize the Cas endonuclease, such that the first and second nucleotide sequence are not found linked together in nature.

The guide polynucleotide can be a double molecule (also referred to as duplex guide polynucleotide) comprising a crNucleotide sequence (such as a crRNA) and a tracrNucleotide (such as a tracrRNA) sequence. The guide polynucleotide can also be a single molecule (also referred to as single guide polynucleotide) comprising a crNucleotide sequence linked to a tracrNucleotide sequence. The single guide polynucleotide comprises a first nucleotide sequence domain (referred to as Variable Targeting domain or VT domain) that can hybridize to a nucleotide sequence in a target DNA and a Cas endonuclease recognition domain (CER domain), that interacts with a Cas endonuclease polypeptide.

The process for editing a genomic sequence at a Cas-gRNA double-strand-break site with a modification template generally comprises: providing a host cell with a Cas-gRNA system, either as individual components or as a complex, that recognizes a target sequence in the genome of the host cell and is able to induce a double-strand-break in the genomic sequence. The Cas endonuclease recognizes a “PAM” (Protospacer Adjacent Motif) on the target strand, and cleaves at or near the target site. Genome editing using double-strand-break-inducing agents, such as Cas9-gRNA complexes, has been described, for example in US20150082478 published on 19 Mar. 2015, WO2015026886 published on 26 Feb. 2015, WO2016007347 published 14 Jan. 2016, and WO2016025131 published on 18 Feb. 2016.

A guide polynucleotide/Cas endonuclease complex that can cleave both strands of a DNA target sequence typically comprises a Cas protein that has all of its endonuclease domains in a functional state (e.g., wild type endonuclease domains or variants thereof retaining some or all activity in each endonuclease domain). Thus, a wild type Cas protein (e.g., a Cas protein disclosed herein), or a variant thereof retaining some or all activity in each endonuclease domain of the Cas protein, is a suitable example of a Cas endonuclease that can cleave both strands of a DNA target sequence.

A guide polynucleotide/Cas endonuclease complex that can cleave one strand of a DNA target sequence can be characterized herein as having nickase activity (e.g., partial cleaving capability). A Cas nickase typically comprises one functional endonuclease domain that allows the Cas to cleave only one strand (i.e., make a nick) of a DNA target sequence. For example, a Cas9 nickase may comprise (i) a mutant, dysfunctional RuvC domain and (ii) a functional HNH domain (e.g., wild type HNH domain). As another example, a Cas9 nickase may comprise (i) a functional RuvC domain (e.g., wild type RuvC domain) and (ii) a mutant, dysfunctional HNH domain. Non-limiting examples of Cas9 nickases suitable for use herein are disclosed in US20140189896 published on 3 Jul. 2014. A pair of Cas nickases can be used to increase the specificity of DNA targeting. In general, this can be done by providing two Cas nickases that, by virtue of being associated with RNA components with different guide sequences, target and nick nearby DNA sequences on opposite strands in the region for desired targeting. Such nearby cleavage of each DNA strand creates a double-strand break (i.e., a DSB with single-stranded overhangs), which is then recognized as a substrate for non-homologous-end-joining, NHEJ (prone to imperfect repair leading to mutations) or homologous recombination, HR.

To facilitate optimal expression and nuclear localization for eukaryotic cells, the gene comprising the Cas endonuclease may be optimized as described in WO2016186953 published 24 Nov. 2016, and then delivered into cells as DNA expression cassettes by methods known in the art. In some aspects, the Cas endonuclease is provided as a polypeptide. In some aspects, the Cas endonuclease is provided as a polynucleotide encoding a polypeptide. In some aspects, the guide RNA is provided as a DNA molecule encoding one or more RNA molecules. In some aspects, the guide RNA is provide as RNA or chemically-modified RNA. In some aspects, the Cas endonuclease protein and guide RNA are provided as a ribonucleoprotein complex (RNP).

A Cas endonuclease, effector protein, or functional fragment thereof, for use in the disclosed methods, can be isolated from a native source, or from, a recombinant source where the genetically modified host cell is modified to express the nucleic acid sequence encoding the protein. Alternatively, the Cas protein can be produced using cell free protein expression systems, or be synthetically produced. Effector Cas nucleases may be isolated and introduced into a heterologous cell, or may be modified from its native form to exhibit a different type or magnitude of activity than what it would exhibit in its native source. Such modifications include but are not limited to: fragments, variants, substitutions, deletions, and insertions.

Fragments and variants of Cas endonucleases and Cas effector proteins can be obtained via methods such as site-directed mutagenesis and synthetic construction. Methods for measuring endonuclease activity are well known in the art such as, but not limiting to, WO2013166113 published 7 Nov. 2013, WO2016186953 published 24 Nov. 2016, and WO2016186946 published 24 Nov. 2016.

The Cas endonuclease can comprise a modified form of the Cas polypeptide. The modified form of the Cas polypeptide can include an amino acid change (e.g., deletion, insertion, or substitution) that reduces the naturally occurring nuclease activity of the Cas protein. For example, in some instances, the modified form of the Cas protein has less than 50%, less than 40%, less than 30%, less than 20%, less than 10%, less than 5%, or less than 1% of the nuclease activity of the corresponding wild-type Cas polypeptide (US20140068797 published 6 Mar. 2014). In some cases, the modified form of the Cas polypeptide has no substantial nuclease activity and is referred to as catalytically “inactivated Cas” or “deactivated Cas (dCas).” An inactivated Cas/deactivated Cas includes a deactivated Cas endonuclease (dCas). A catalytically inactive Cas effector protein can be fused to a heterologous sequence to induce or modify activity.

A Cas endonuclease can be part of a fusion protein comprising one or more heterologous protein domains (e.g., 1, 2, 3, or more domains in addition to the Cas protein). Such a fusion protein may comprise any additional protein sequence, and optionally a linker sequence between any two domains, such as between Cas and a first heterologous domain. Examples of protein domains that may be fused to a Cas protein herein include, without limitation, epitope tags (e.g., histidine [His], V5, FLAG, influenza hemagglutinin [HA], myc, VSV-G, thioredoxin [Trx]), reporters (e.g., glutathione-5-transferase [GST], horseradish peroxidase [RP], chloramphenicol acetyltransferase [CAT], beta-galactosidase, beta-glucuronidase [GUS], luciferase, green fluorescent protein [GFP], HcRed, DsRed, cyan fluorescent protein [CFP], yellow fluorescent protein [YFP], blue fluorescent protein [BFP]), and domains having one or more of the following activities: methylase activity, demethylase activity, transcription activation activity (e.g., VP16 or VP64), transcription repression activity, transcription release factor activity, histone modification activity, RNA cleavage activity and nucleic acid binding activity. A Cas protein can also be in fusion with a protein that binds DNA molecules or other molecules, such as maltose binding protein (MBP), S-tag, Lex A DNA binding domain (DBD), GAL4A DNA binding domain, and herpes simplex virus (HSV) VP16.

A catalytically active and/or inactive Cas endonuclease can be fused to a heterologous sequence (US20140068797 published 6 Mar. 2014). Suitable fusion partners include, but are not limited to, a polypeptide that provides an activity that indirectly increases transcription by acting directly on the target DNA or on a polypeptide (e.g., a histone or other DNA-binding protein) associated with the target DNA. Additional suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity. Further suitable fusion partners include, but are not limited to, a polypeptide that directly provides for increased transcription of the target nucleic acid (e.g., a transcription activator or a fragment thereof, a protein or fragment thereof that recruits a transcription activator, a small molecule/drug-responsive transcription regulator, etc.). A partially active or catalytically inactive Cas endonuclease can also be fused to another protein or domain, for example Clo51 or FokI nuclease, to generate double-strand breaks (Guilinger et al. Nature Biotechnology, volume 32, number 6, June 2014).

### Morphogenic Factors

The present disclosure comprises methods and compositions for producing genomic modifications in an organism using a double-strand-break agent and a morphogenic factor. Morphogenic factors can enhance the rate, efficiency, and efficacy of targeted polynucleotide modification by a number of mechanisms, some of which are related to the capability of stimulating growth of a cell or tissue, including but not limited to: promoting progression through the cell cycle, inhibiting cell death, such as apoptosis, stimulating cell division, and/or stimulating embryogenesis. The polynucleotides can fall into several categories, including but not limited to, cell cycle stimulatory polynucleotides, developmental polynucleotides, anti-apoptosis polynucleotides, hormone polynucleotides, transcription factors, or silencing constructs targeted against cell cycle repressors or pro-apoptotic factors. Methods and compositions for rapid and efficient transformation of plants by transforming cells of plant explants with an expression construct comprising a heterologous nucleotide encoding a morphogenic factor are described in US Patent Application Publication No. US2017/0121722 (published 4 May 2017). A suitable plant explant includes for example, a mature seed, seed-derived mature embryos, apical meristems, axillary meristems, leaf tissue, leaf bases, stems, vascular cambium, the immature inflorescence, immature ears, immature tassels, immature embryos, immature cotyledons, mature or imbibed cotyledons, imbibed seed or seed-derived parts. The above explants can be derived from a diploid 2N plant or a haploid 1N plant or a plant part.

A morphogenic factor (gene or protein) may involved in plant metabolism, organ development, stem cell development, cell growth stimulation, organogenesis, somatic embryogenesis initiation, accelerated somatic embryo maturation, initiation and/or development of the apical meristem, initiation and/or development of shoot meristem, or a combination thereof.

In some aspects, the morphogenic factor is a molecule selected from one or more of the following categories: 1) cell cycle stimulatory polynucleotides including plant viral replicase genes such as RepA, cyclins, E2F, prolifera, cdc2 and cdc25; 2) developmental polynucleotides such as Lec1, Kn1 family, WUSCHEL, Zwille, BBM, Aintegumenta (ANT), FUS3, and members of the Knotted family, such as Kn1, STM, OSH1, and SbHl; 3) anti-apoptosis polynucleotides such as CED9, Bcl2, Bcl-X(L), Bcl-W, A1, McL-1, Mac1, Boo, and Bax-inhibitors; 4) hormone polynucleotides such as IPT, TZS, and CKI-1; and 5) silencing constructs targeted against cell cycle repressors, such as Rb, CK1, prohibitin, and weel, or stimulators of apoptosis such as APAF-1, bad, bax, CED-4, and caspase-3, and repressors of plant developmental transitions, such as Pickle and WD polycomb genes including FIE and Medea. The polynucleotides can be silenced by any known method such as antisense, RNA interference, cosuppression, chimerplasty, or transposon insertion.

In some aspects, the morphogenic gene is a member of the WUS/WOX gene family (WUS1, WUS2, WUS3, WOX2A, WOX4, WOX5, or WOX9) see U.S. Pat. Nos. 7,348,468 and 7,256,322 and United States Patent Application publications 20170121722 and 20070271628. Modulation of WUS/WOX is expected to modulate plant and/or plant tissue phenotype including plant metabolism, organ development, stem cell development, cell growth stimulation, organogenesis, somatic embryogenesis initiation, accelerated somatic embryo maturation, initiation and/or development of the apical meristem, initiation and/or development of shoot meristem, or a combination thereof. Expression of Arabidopsis WUS can induce stem cells in vegetative tissues, which can differentiate into somatic embryos (Zuo, et al. (2002) Plant J 30:349-359). Also of interest in this regard would be a MYB118 gene (see U.S. Pat. No. 7,148,402), MYB 115 gene (see Wang et al. (2008) Cell Research 224-235), a BABYBOOM gene (BBM; see Boutilier et al. (2002) Plant Cell 14:1737-1749), or a CLAVATA gene (see, for example, U.S. Pat. No. 7,179,963).

In some embodiments, the morphogenic gene or protein is a member of the AP2/ERF family of proteins. The AP2/ERF family of proteins is a plant-specific class of putative transcription factors that regulate a wide variety of developmental processes and are characterized by the presence of an AP2 DNA binding domain that is predicted to form an amphipathic alpha helix that binds DNA (PFAM Accession PF00847).

Members of the APETALA2 (AP2) family of proteins function in a variety of biological events, including but not limited to, development, plant regeneration, cell division, embryogenesis, and morphogenic (see, e.g., Riechmann and Meyerowitz (1998) Biol Chem 379:633-646; Saleh and Pages (2003) Genetika 35:37-50 and Database of Arabidopsis Transciption Factors at daft.cbi.pku.edu.cn). The AP2 family includes, but is not limited to, AP2, ANT, Glossy15, AtBBM, BnBBM, and maize ODP2/BBM-morphogenic genes.

Other morphogenic genes useful in the present disclosure include, but are not limited to, Ovule Development Protein 2 (ODP2) polypeptides, and related polypeptides, e.g., Babyboom (BBM) protein family proteins. In an aspect, the polypeptide comprising the two AP2-DNA binding domains is an ODP2, BBM2, BMN2, or BMN3 polypeptide. The ODP2 polypeptides of the disclosure contain two predicted APETALA2 (AP2) domains and are members of the AP2 protein family (PFAM Accession PF00847). The AP2 domain has now been found in a variety of proteins. The ODP2 polypeptides share homology with several polypeptides within the AP2 family, e.g., see FIG. 1 of U.S. Pat. No. 8,420,893, which is incorporated herein by reference in its entirety, provides an alignment of the maize and rice ODP2 polypeptides with eight other proteins having two AP2 domains.

In some embodiments, the morphogenic factor is a babyboom (BBM) polypeptide, which is a member of the AP2 family of transcription factors. The BBM protein from Arabidopsis (AtBBM) is preferentially expressed in the developing embryo and seeds and has been shown to play a central role in regulating embryo-specific pathways. Overexpression of AtBBM has been shown to induce spontaneous formation of somatic embryos and cotyledon-like structures on seedlings. See, Boutiler et al. (2002) The Plant Cell 14:1737-1749. The maize BBM protein also induces embryogenesis and promotes transformation (See, U.S. Pat. No. 7,579,529, which is herein incorporated by reference in its entirety). Thus, BBM polypeptides stimulate proliferation, induce embryogenesis, enhance the regenerative capacity of a plant, enhance transformation, and as demonstrated herein, enhance rates of targeted polynucleotide modification. As used herein “regeneration” refers to a morphogenic response that results in the production of new tissues, organs, embryos, whole plants or parts of whole plants that are derived from a single cell or a group of cells. Regeneration may proceed indirectly via a callus phase or directly, without an intervening callus phase. “Regenerative capacity” refers to the ability of a plant cell to undergo regeneration.

Other morphogenic genes useful in the present disclosure include, but are not limited to, LEC1 (Lotan et al., 1998, Cell 93:1195-1205), LEC2 (Stone et al., 2008, PNAS 105:3151-3156; Belide et al., 2013, Plant Cell Tiss. Organ Cult 113:543-553), KN1/STM (Sinha et al., 1993. Genes Dev 7:787-795), the IPT gene from Agrobacterium (Ebinuma and Komamine, 2001, In vitro Cell. Dev Biol—Plant 37:103-113), MONOPTEROS-DELTA (Ckurshumova et al., 2014, New Phytol. 204:556-566), the Agrobacterium AV-6b gene (Wabiko and Minemura 1996, Plant Physiol. 112:939-951), the combination of the Agrobacterium IAA-h and IAA-m genes (Endo et al., 2002, Plant Cell Rep., 20:923-928), the Arabidopsis SERK gene (Hecht et al., 2001, Plant Physiol. 127:803-816), the Arabiopsis AGL15 gene (Harding et al., 2003, Plant Physiol. 133:653-663), and the FUSCA gene (Castle and Meinke, Plant Cell 6:25-41), and the PICKLE gene (Ogas et al., 1999, PNAS 96:13839-13844).

The morphogenic factor can be derived from a monocot. In various aspects, the morphogenic factor is derived from barley, maize, millet, oats, rice, rye, Setaria sp., sorghum, sugarcane, switchgrass, triticale, turfgrass, or wheat.

The morphogenic factor can be derived from a dicot. The morphogenic factor can be derived from kale, cauliflower, broccoli, mustard plant, cabbage, pea, clover, alfalfa, broad bean, tomato, cassava, soybean, canola, alfalfa, sunflower, safflower, tobacco, Arabidopsis, poplar, apple, Amaranthus, or cotton. The morphogenic factor can also be derived from a gymnosperm, more specifically from a species within the Cycadophyta (with examples such as Encephalartos spp, Cycas, spp, Bownenia spp, and Macrozamia spp.), Ginkgophyta (with examples such as Ginko biloba), Gnetophyta (with examples such as Gnetum gnomen, Ephedra spp. and Welwitschia spp), and Pinophyta (with examples such as Pinus spp, Abies spp, Agathis spp, Tsuga spp, Pseudotsuga menziesii, Chamaecyparis spp, Juniperus spp, Taxodium spp, Cupressus spp, Metasequoia, and Sequoia).

The present disclosure encompasses isolated or substantially purified polynucleotide or polypeptide morphogenic factor compositions.

The morphogenic factor may be altered in various ways including amino acid substitutions, deletions, truncations, and insertions. Methods for such manipulations are generally known. For example, amino acid sequence variants of the morphogenic proteins can be prepared by mutations in the DNA. Methods for mutagenesis and nucleotide sequence alterations are well known in the art. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

In some aspects, the morphogenic gene is selected from the group consisting of: SEQ ID NOs:1-5, 11-16, 22, and 23-47. In some aspects, the morphogenic protein is selected from the group consisting of: SEQ ID NOs: 6-10, 17-21, and 48-73.

In some aspects, a plurality of morphogenic factors is selected. When multiple morphogenic factors are used, the polynucleotides encoding each of the factors can be present on the same expression cassette or on separate expression cassettes. Likewise, the polynucleotide(s) encoding the morphogenic factor(s) and the polynucleotide encoding the double-strand break-inducing agent can be located on the same or different expression cassettes. When two or more factors are coded for by separate expression cassettes, the expression cassettes can be provided to the organism simultaneously or sequentially.

In some aspects, the expression of the morphogenic factor is transient. In some aspects, the expression of the morphogenic factor is constitutive. In some aspects, the expression of the morphogenic factor is specific to a particular tissue or cell type. In some aspects, the expression of the morphogenic factor is temporally regulated. In some aspects, the expression of the morphogenic factor is regulated by an environmental condition, such as temperature, time of day, or other factor. In some aspects, the expression of the morphogenic factor is stable. In some aspects, expression of the morphogenic factor is controlled. The controlled expression may be a pulsed expression of the morphogenic factor for a particular period of time. Alternatively, the morphogenic factor may be expressed in only some transformed cells and not expressed in others. The control of expression of the morphogenic factor can be achieved by a variety of methods as disclosed herein.

### Polynucleotides and Plant Traits of Interest

Polynucleotides of interest are further described herein and include polynucleotides reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for genetic engineering will change accordingly.

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802, and 5,990,389.

Polynucleotide sequences of interest may encode proteins involved in providing disease or pest resistance. By “disease resistance” or “pest resistance” is intended that the plants avoid the harmful symptoms that are the outcome of the plant-pathogen interactions. Pest resistance genes may encode resistance to pests that have great yield drag such as rootworm, cutworm, European Corn Borer, and the like. Disease resistance and insect resistance genes such as lysozymes or cecropins for antibacterial protection, or proteins such as defensins, glucanases or chitinases for antifungal protection, or Bacillus thuringiensis endotoxins, protease inhibitors, collagenases, lectins, or glycosidases for controlling nematodes or insects are all examples of useful gene products. Genes encoding disease resistance traits include detoxification genes, such as against fumonisin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like. Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser et al. (1986) Gene 48:109); and the like.

An “herbicide resistance protein” or a protein resulting from expression of an “herbicide resistance-encoding nucleic acid molecule” includes proteins that confer upon a cell the ability to tolerate a higher concentration of an herbicide than cells that do not express the protein, or to tolerate a certain concentration of an herbicide for a longer period of time than cells that do not express the protein. Herbicide resistance traits may be introduced into plants by genes coding for resistance to herbicides that act to inhibit the action of acetolactate synthase (ALS, also referred to as acetohydroxyacid synthase, AHAS), in particular the sulfonylurea (UK: sulphonylurea) type herbicides, genes coding for resistance to herbicides that act to inhibit the action of glutamine synthase, such as phosphinothricin or basta (e.g., the bar gene), glyphosate (e.g., the EPSP synthase gene and the GAT gene), HPPD inhibitors (e.g, the HPPD gene) or other such genes known in the art. See, for example, U.S. Pat. Nos. 7,626,077; 5,310,667; 5,866,775; 6,225,114, 6,248,876; 7,169,970; 6,867,293, and 9,187,762. The bar gene encodes resistance to the herbicide basta, the nptII gene encodes resistance to the antibiotics kanamycin and geneticin, and the ALS-gene mutants encode resistance to the herbicide chlorsulfuron.

Furthermore, it is recognized that the polynucleotide of interest may also comprise antisense sequences complementary to at least a portion of the messenger RNA (mRNA) for a targeted gene sequence of interest. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, 80%, or 85% sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides, or greater may be used.

In addition, the polynucleotide of interest may also be used in the sense orientation to suppress the expression of endogenous genes in plants. Methods for suppressing gene expression in plants using polynucleotides in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, generally greater than about 65% sequence identity, about 85% sequence identity, or greater than about 95% sequence identity. See U.S. Pat. Nos. 5,283,184 and 5,034,323.

The polynucleotide of interest can also be a phenotypic marker. A phenotypic marker is screenable or a selectable marker that includes visual markers and selectable markers whether it is a positive or negative selectable marker. Any phenotypic marker can be used. Specifically, a selectable or screenable marker comprises a DNA segment that allows one to identify, or select for or against a molecule or a cell that comprises it, often under particular conditions. These markers can encode an activity, such as, but not limited to, production of RNA, peptide, or protein, or can provide a binding site for RNA, peptides, proteins, inorganic and organic compounds or compositions and the like.

Examples of selectable markers include, but are not limited to, DNA segments that comprise restriction enzyme sites; DNA segments that encode products which provide resistance against otherwise toxic compounds including antibiotics, such as, spectinomycin, ampicillin, kanamycin, tetracycline, Basta, neomycin phosphotransferase II (NEO) and hygromycin phosphotransferase (HPT)); DNA segments that encode products which are otherwise lacking in the recipient cell (e.g., tRNA genes, auxotrophic markers); DNA segments that encode products which can be readily identified (e.g., phenotypic markers such as 0-galactosidase, GUS; fluorescent proteins (such as green fluorescent protein (GFP), cyan (CFP), yellow (YFP), red (RFP), and blue (BFP), (Shaner et al., 2005, Nature Methods 2:905-909)), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying agent, chemical, etc.; and, the inclusion of a DNA sequences required for a specific modification (e.g., methylation) that allows its identification.

Additional selectable markers include genes that confer resistance to herbicidal compounds, such as sulphonylureas, glufosinate ammonium, bromoxynil, imidazolinones, and 2,4-dichlorophenoxyacetate (2,4-D). See for example, Acetolactase synthase (ALS) for resistance to sulfonylureas, imidazolinones, triazolopyrimidine sulfonamides, pyrimidinylsalicylates and sulphonylaminocarbonyl-triazolinones (Shaner and Singh, 1997, Herbicide Activity: Toxicol Biochem Mol Biol 69-110); glyphosate resistant 5-enolpyruvylshikimate-3-phosphate (EPSPS) (Saroha et al. 1998, J. Plant Biochemistry & Biotechnology Vol 7:65-72).

### Recombinant Constructs for Transformation of Cells

The disclosed guide polynucleotides, Cas endonucleases, polynucleotide modification templates, donor DNAs, guide polynucleotide/Cas endonuclease systems disclosed herein, and any one combination thereof, optionally further comprising one or more polynucleotide(s) of interest, can be introduced into a cell. Cells include, but are not limited to, human, non-human, animal, bacterial, fungal, insect, yeast, non-conventional yeast, and plant cells as well as pollen, plants and seeds produced by the methods described herein.

Standard recombinant DNA and molecular cloning techniques used herein are well known in the art and are described more fully in Sambrook et al., Molecular Cloning: A Laboratory Manual; Cold Spring Harbor Laboratory: Cold Spring Harbor, NY (1989). Transformation methods are well known to those skilled in the art and are described infra.

Vectors and constructs include circular plasmids, and linear polynucleotides, comprising a polynucleotide of interest and optionally other components including linkers, adapters, regulatory or analysis. In some examples a recognition site and/or target site can be comprised within an intron, coding sequence, 5′ UTRs, 3′ UTRs, and/or regulatory regions.

Various methods and compositions can be employed to obtain a cell or organism having a polynucleotide of interest inserted in a target site for a Cas endonuclease. Such methods can employ homologous recombination (HR) to provide integration of the polynucleotide of interest at the target site. In one method described herein, a polynucleotide of interest is introduced into the organism cell via a donor DNA construct. The donor DNA construct further comprises a first and a second region of homology that flank the polynucleotide of interest. The first and second regions of homology of the donor DNA share homology to a first and a second genomic region, respectively, present in or flanking the target site of the cell or organism genome.

The donor DNA can be tethered to the guide polynucleotide. Tethered donor DNAs can allow for co-localizing target and donor DNA, useful in genome editing, gene insertion, and targeted genome regulation, and can also be useful in targeting post-mitotic cells where function of endogenous HR machinery is expected to be highly diminished (Mali et al., 2013, Nature Methods Vol. 10: 957-963).

The amount of homology or sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, between 98% and 99%, 99%, between 99% and 100%, or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, New York).

### Optimization of Sequences for Expression in Plants

Methods are available in the art for synthesizing plant-preferred genes. See, for example, U.S. Pat. Nos. 5,380,831, and 5,436,391, and Murray et al. (1989) Nucleic Acids Res. 17:477-498. Additional sequence modifications are known to enhance gene expression in a plant host. These include, for example, elimination of: one or more sequences encoding spurious polyadenylation signals, one or more exon-intron splice site signals, one or more transposon-like repeats, and other such well-characterized sequences that may be deleterious to gene expression. The G-C content of the sequence may be adjusted to levels average for a given plant host, as calculated by reference to known genes expressed in the host plant cell. When possible, the sequence is modified to avoid one or more predicted hairpin secondary mRNA structures. Thus, “a plant-optimized nucleotide sequence” of the present disclosure comprises one or more of such sequence modifications.

### Expression Elements

Any polynucleotide encoding a Cas protein or other CRISPR system component disclosed herein may be functionally linked to a heterologous expression element, to facilitate transcription or regulation in a host cell. Such expression elements include but are not limited to: promoter, leader, intron, and terminator. Expression elements may be “minimal”—meaning a shorter sequence derived from a native source, that still functions as an expression regulator or modifier. Alternatively, an expression element may be “optimized”—meaning that its polynucleotide sequence has been altered from its native state in order to function with a more desirable characteristic in a particular host cell. Alternatively, an expression element may be “synthetic”—meaning that it is designed in silico and synthesized for use in a host cell. Synthetic expression elements may be entirely synthetic, or partially synthetic (comprising a fragment of a naturally-occurring polynucleotide sequence).

It has been shown that certain promoters are able to direct RNA synthesis at a higher rate than others. These are called “strong promoters”. Certain other promoters have been shown to direct RNA synthesis at higher levels only in particular types of cells or tissues and are often referred to as “tissue specific promoters”, or “tissue-preferred promoters” if the promoters direct RNA synthesis preferably in certain tissues but also in other tissues at reduced levels.

A plant promoter includes a promoter capable of initiating transcription in a plant cell. For a review of plant promoters, see, Potenza et al., 2004, In vitro Cell Dev Biol 40:1-22; Porto et al., 2014, Molecular Biotechnology (2014), 56(1), 38-49.

Constitutive promoters include, for example, the core CaMV 35S promoter (Odell et al., (1985) Nature 313:810-2); rice actin (McElroy et al., (1990) Plant Cell 2:163-71); ubiquitin (Christensen et al., (1989) Plant Mol Biol 12:619-32; ALS promoter (U.S. Pat. No. 5,659,026) and the like.

Tissue-preferred promoters can be utilized to target enhanced expression within a particular plant tissue. Tissue-preferred promoters include, for example, WO2013103367 published 11 Jul. 2013, Kawamata et al., (1997) Plant Cell Physiol 38:792-803; Hansen et al., (1997) Mol Gen Genet 254:337-43; Russell et al., (1997) Transgenic Res 6:157-68; Rinehart et al., (1996) Plant Physiol 112:1331-41; Van Camp et al., (1996) Plant Physiol 112:525-35; Canevascini et al., (1996) Plant Physiol 112:513-524; Lam, (1994) Results Probl Cell Differ 20:181-96; and Guevara-Garcia et al., (1993) Plant J4:495-505. Leaf-preferred promoters include, for example, Yamamoto et al., (1997) Plant J 12:255-65; Kwon et al., (1994) Plant Physiol 105:357-67; Yamamoto et al., (1994) Plant Cell Physiol 35:773-8.

Seed-preferred promoters include both seed-specific promoters active during seed development, as well as seed-germinating promoters active during seed germination. See, Thompson et al., (1989) BioEssays 10:108. Seed-preferred promoters include, but are not limited to, Cim1 (cytokinin-induced message); cZ19B1 (maize 19 kDa zein); and milps (myo-inositol-1-phosphate synthase); and for example, those disclosed in WO2000011177 published 2 Mar. 2000 and U.S. Pat. No. 6,225,529. For dicots, seed-preferred promoters include, but are not limited to, bean β-phaseolin, napin, β-conglycinin, soybean lectin, cruciferin, and the like. For monocots, seed-preferred promoters include, but are not limited to, maize 15 kDa zein, 22 kDa zein, 27 kDa gamma zein, waxy, shrunken 1, shrunken 2, globulin 1, oleosin, and nuc1. See also, WO2000012733 published 9 Mar. 2000, where seed-preferred promoters from END1 and END2 genes are disclosed.

Chemical inducible (regulated) promoters can be used to modulate the expression of a gene in a prokaryotic and eukaryotic cell or organism through the application of an exogenous chemical regulator. The promoter may be a chemical-inducible promoter, where application of the chemical induces gene expression, or a chemical-repressible promoter, where application of the chemical represses gene expression. Chemical-inducible promoters include, but are not limited to, the maize In2-2 promoter, activated by benzene sulfonamide herbicide safeners (De Veylder et al., (1997) Plant Cell Physiol 38:568-77), the maize GST promoter (GST-II-27, WO1993001294 published 21 Jan. 1993), activated by hydrophobic electrophilic compounds used as pre-emergent herbicides, and the tobacco PR-1a promoter (Ono et al., (2004) Biosci Biotechnol Biochem 68:803-7) activated by salicylic acid. Other chemical-regulated promoters include steroid-responsive promoters (see, for example, the glucocorticoid-inducible promoter (Schena et al., (1991) Proc. Natl. Acad. Sci. USA 88:10421-5; McNellis et al., (1998) Plant J 14:247-257); tetracycline-inducible and tetracycline-repressible promoters (Gatz et al., (1991)Mol Gen Genet 227:229-37; U.S. Pat. Nos. 5,814,618 and 5,789,156).

Another example of an inducible promoter useful in plant cells, is the ZmCAS1 promoter, described in US20130312137 published 21 Nov. 2013. New promoters of various types useful in plant cells are constantly being discovered; numerous examples may be found in the compilation by Okamuro and Goldberg, (1989) In The Biochemistry ofPlants, Vol. 115, Stumpf and Conn, eds (New York, NY: Academic Press), pp. 1-82.

### Polynucleotide Constructs

One or more of the polynucleotide compositions herein may be provided to a cell as part of a expression construct, plasmid, or vector. Said construct may comprise one or more expression cassettes for transcription in the cell.

The transcriptional cassette will include in the 5′-3′ direction of transcription, a transcriptional and translational initiation region, a DNA sequence of interest, and a transcriptional and translational termination region functional in plants. The expression cassettes may contain one or more than one gene or nucleic acid sequence to be transferred and expressed in the transformed plant. Thus, each nucleic acid sequence will be operably linked to 5′ and 3′ regulatory sequences. Alternatively, multiple expression cassettes may be provided.

### Modification of a Target Polynucleotide in a Plant Cell

According to the presently disclosed methods, morphogenic factors are used to enhance the modification of a target site polynucleotide within a cell that is effected by DSB agent activity. Once a single or double-strand break is induced in the DNA, the cell's DNA repair mechanism is activated to repair the break. Error-prone DNA repair mechanisms can produce mutations at double-strand break sites. The most common repair mechanism to bring the broken ends together is the nonhomologous end-joining (NHEJ) pathway (Bleuyard et al., (2006) DNA Repair 5:1-12).

Homology-directed repair (HDR) is a mechanism in cells to repair double-stranded and single stranded DNA breaks. Homology-directed repair includes homologous recombination (HR) and single-strand annealing (SSA) (Lieber. 2010 Annu. Rev. Biochem. 79:181-211). The most common form of HDR is called homologous recombination (HR), which has the longest sequence homology requirements between the donor and acceptor DNA. Other forms of HDR include single-stranded annealing (SSA) and breakage-induced replication, and these require shorter sequence homology relative to HR. Homology-directed repair at nicks (single-stranded breaks) can occur via a mechanism distinct from HDR at double-strand breaks (Davis and Maizels. PNAS (0027-8424), 111 (10), p. E924-E932).

Alteration of the genome of a prokaryotic and eukaryotic cell or organism cell, for example, through homologous recombination (HR), is a powerful tool for genetic engineering.

### Target Sites

The target site may be part of the organism's native genome or integrated therein or may be present on an episomal polynucleotide. The genomic target sequence may be on any region of any chromosome, and may or may not be in a region encoding a protein or RNA. The target site may be native to the cell or heterologous. In some embodiments, the heterologous target sequence may have been transgenically inserted into the organism's genome, and may be on any region of any chromosome, including an artificial or satellite chromosome, and may or may not be in a region encoding a protein or RNA. In some aspects, the target site polynucleotide is of nuclear origin (genomic), and may be either endogenous to the cell or may be heterologous (e.g. an introduced transgene). In some aspects, the target site polynucleotide is a plasmid or vector that exists within the cell or has been introduced. In some aspects, the target site polynucleotide exists in the cytoplasm of the cell (extra-nuclear). In some aspects, the target site polynucleotide exists in another organelle of the cell (e.g. plastid or mitochondrion).It is recognized that the cell or the organism may comprise multiple target sites, which may be located at one or multiple loci within or across chromosomes. Multiple independent manipulations of each target site in the organism can be performed using the presently disclosed methods.

The target site comprises at least one recognition sequence. The length of the recognition site sequence can vary, and includes, for example, sequences that are at least about 3, 4, 6, 8, 10, 12, 14, 16, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 80, 90, 100, or more nucleotides in length. In some embodiments, the recognition site is of a sufficient length to only be present in a genome of an organism one time. In some embodiments, the recognition site is palindromic, that is, the sequence on one strand reads the same in the opposite direction on the complementary strand. The double-strand break-inducing agent recognizes the recognition sequence and introduces a double-strand break at or near the recognition sequence. The nick/cleavage site could be within the sequence that is specifically recognized by the agent or the nick/cleavage site could be outside of the sequence that is specifically recognized by the agent. In some embodiments, the double-strand break is introduced about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, 200, or more nucleotides away from the recognition sequence.

In some embodiments, the cleavage occurs at nucleotide positions immediately opposite each other to produce a blunt end cut or, in alternative embodiments, the cuts are staggered to produce single-stranded overhangs, also called “sticky ends”, which can be either 5′ overhangs, or 3′ overhangs. The recognition sequence can be endogenous (native) or heterologous to the plant cell. When the recognition site is an endogenous sequence, it may be recognized by a naturally-occurring, or native double-strand break-inducing agent. Alternatively, an endogenous recognition sequence may be recognized and/or bound by a modified or engineered double-strand break-inducing agent designed or selected to specifically recognize the endogenous recognition sequence to produce a double-strand break.

### Gene Targeting

The guide polynucleotide/Cas systems described herein can be used for gene targeting.

In general, DNA targeting can be performed by cleaving one or both strands at a specific polynucleotide sequence in a cell with a Cas protein associated with a suitable polynucleotide component. Once a single or double-strand break is induced in the DNA, the cell's DNA repair mechanism is activated to repair the break via nonhomologous end-joining (NHEJ) or Homology-Directed Repair (HDR) processes which can lead to modifications at the target site.

The length of the DNA sequence at the target site can vary, and includes, for example, target sites that are at least 12, 13, 14, 15, 16, 17, 18, 19, 20, 21, 22, 23, 24, 25, 26, 27, 28, 29, 30, or more than 30 nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other cases, the incisions could be staggered to produce single-stranded overhangs, also called “sticky ends” or “staggered end”, which can be either 5′ overhangs, or 3′ overhangs. Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by a Cas endonuclease.

Assays to measure the single or double-strand break of a target site by an endonuclease are known in the art and generally measure the overall activity and specificity of the agent on DNA substrates comprising recognition sites.

A targeting method herein can be performed in such a way that two or more DNA target sites are targeted in the method, for example. Such a method can optionally be characterized as a multiplex method. Two, three, four, five, six, seven, eight, nine, ten, or more target sites can be targeted at the same time in certain embodiments. A multiplex method is typically performed by a targeting method herein in which multiple different RNA components are provided, each designed to guide a guide polynucleotide/Cas endonuclease complex to a unique DNA target site.

The process for editing a genomic sequence combining DSB and modification templates generally comprises: introducing into a host cell a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB. Genome editing using DSB-inducing agents, such as Cas-gRNA complexes, has been described, for example in US20150082478 published on 19 Mar. 2015, WO2015026886 published on 26 Feb. 2015, WO2016007347 published 14 Jan. 2016, and WO2016025131 published on 18 Feb. 2016.

Some uses for guide RNA/Cas endonuclease systems have been described (see for example: US20150082478A1 published 19 Mar. 2015, WO2015026886 published 26 Feb. 2015, and US20150059010 published 26 Feb. 2015) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

Proteins may be altered in various ways including amino acid substitutions, deletions, truncations, and insertions. Methods for such manipulations are generally known. For example, amino acid sequence variants of the protein(s) can be prepared by mutations in the DNA. Methods for mutagenesis and nucleotide sequence alterations include, for example, Kunkel, (1985) Proc. Natl. Acad. Sci. USA 82:488-92; Kunkel et al., (1987) Meth Enzymol 154:367-82; U.S. Pat. No. 4,873,192; Walker and Gaastra, eds. (1983) Techniques in Molecular Biology (MacMillan Publishing Company, New York) and the references cited therein. Guidance regarding amino acid substitutions not likely to affect biological activity of the protein is found, for example, in the model of Dayhoff et al., (1978) Atlas of Protein Sequence and Structure (Natl Biomed Res Found, Washington, D.C.). Conservative substitutions, such as exchanging one amino acid with another having similar properties, may be preferable. Conservative deletions, insertions, and amino acid substitutions are not expected to produce radical changes in the characteristics of the protein, and the effect of any substitution, deletion, insertion, or combination thereof can be evaluated by routine screening assays. Assays for double-strand-break-inducing activity are known and generally measure the overall activity and specificity of the agent on DNA substrates comprising target sites.

Described herein are methods for genome editing with CRISPR Associated (Cas) endonucleases. Following characterization of the guide RNA (or guide polynucleotide) and PAM sequence, a ribonucleoprotein (RNP) complex comprising the Cas endonuclease and the guide RNA (or guide polynucleotide) may be utilized to modify a target polynucleotide, including but not limited to: synthetic DNA, isolated genomic DNA, or chromosomal DNA in other organisms, including plants. To facilitate optimal expression and nuclear localization (for eukaryotic cells), the gene comprising the Cas endonuclease may be optimized, and then delivered into cells as DNA expression cassettes by methods known in the art. The components necessary to comprise an active RNP may also be delivered as RNA with or without modifications that protect the RNA from degradation or as mRNA capped or uncapped (Zhang, Y. et al., 2016, Nat. Commun. 7:12617) or Cas protein guide polynucleotide complexes (WO2017070032 published 27 Apr. 2017), or any combination thereof. Additionally, a part or part(s) of the complex may be expressed from a DNA construct while other components are delivered as RNA with or without modifications that protect the RNA from degradation or as mRNA capped or uncapped (Zhang et al. 2016 Nat. Commun. 7:12617) or Cas protein guide polynucleotide complexes (WO2017070032 published 27 Apr. 2017) or any combination thereof. To produce crRNAs in-vivo, tRNA derived elements may also be used to recruit endogenous RNAses to cleave crRNA transcripts into mature forms capable of guiding the complex to its DNA target site, as described, for example, in WO2017105991 published 22 Jun. 2017. Furthermore, the cleavage activity of the Cas endonuclease may be deactivated by altering key catalytic residues in its cleavage domain (Sinkunas, T. et al., 2013, EMBO J. 32:385-394) resulting in a RNA guided helicase that may be used to enhance homology directed repair, induce transcriptional activation, or remodel local DNA structures. Moreover, the activity of the Cas cleavage and helicase domains may both be knocked-out and used in combination with other DNA cutting, DNA nicking, DNA binding, transcriptional activation, transcriptional repression, DNA remodeling, DNA deamination, DNA unwinding, DNA recombination enhancing, DNA integration, DNA inversion, and DNA repair agents.

The transcriptional direction of the tracrRNA for the CRISPR-Cas system (if present) and other components of the CRISPR-Cas system (such as variable targeting domain, crRNA repeat, loop, anti-repeat) can be deduced as described in WO2016186946 published 24 Nov. 2016, and WO2016186953 published 24 Nov. 2016.

In one embodiment, the invention describes a method for modifying a target site in the genome of a cell, the method comprising introducing into a cell at least one PGEN described herein, and identifying at least one cell that has a modification at said target, wherein the modification at said target site is selected from the group consisting of (i) a replacement of at least one nucleotide, (ii) a deletion of at least one nucleotide, (iii) an insertion of at least one nucleotide, and (iv) any combination of (i)-(iii).

The nucleotide to be edited can be located within or outside a target site recognized and cleaved by a Cas endonuclease. In one embodiment, the at least one nucleotide modification is not a modification at a target site recognized and cleaved by a Cas endonuclease. In another embodiment, there are at least 1, 2, 3, 4, 5, 6, 7, 8, 9, 10, 11, 12, 13, 14, 15, 16, 17, 18, 19, 20, 21, 22, 23, 24, 25, 26, 27, 30, 40, 50, 100, 200, 300, 400, 500, 600, 700, 900 or 1000 nucleotides between the at least one nucleotide to be edited and the genomic target site.

A knock-out may be produced by an indel (insertion or deletion of nucleotide bases in a target DNA sequence through NHEJ), or by specific removal of sequence that reduces or destroys the function of sequence at or near the targeting site.

A guide polynucleotide/Cas endonuclease induced targeted mutation can occur in a nucleotide sequence that is located within or outside a genomic target site that is recognized and cleaved by the Cas endonuclease.

The method for editing a nucleotide sequence in the genome of a cell can be a method without the use of an exogenous selectable marker by restoring function to a non-functional gene product.

In one embodiment, the invention describes a method for modifying a target site in the genome of a cell, the method comprising introducing into a cell at least one PGEN described herein and at least one donor DNA, wherein said donor DNA comprises a polynucleotide of interest, and optionally, further comprising identifying at least one cell that said polynucleotide of interest integrated in or near said target site.

In one aspect, the methods disclosed herein may employ homologous recombination (HR) to provide integration of the polynucleotide of interest at the target site.

Various methods and compositions can be employed to produce a cell or organism having a polynucleotide of interest inserted in a target site via activity of a CRISPR-Cas system component described herein. In one method described herein, a polynucleotide of interest is introduced into the organism cell via a donor DNA construct. As used herein, “donor DNA” is a DNA construct that comprises a polynucleotide of interest to be inserted into the target site of a Cas endonuclease. The donor DNA construct further comprises a first and a second region of homology that flank the polynucleotide of interest. The first and second regions of homology of the donor DNA share homology to a first and a second genomic region, respectively, present in or flanking the target site of the cell or organism genome.

The donor DNA can be tethered to the guide polynucleotide. Tethered donor DNAs can allow for co-localizing target and donor DNA, useful in genome editing, gene insertion, and targeted genome regulation, and can also be useful in targeting post-mitotic cells where function of endogenous HR machinery is expected to be highly diminished (Mali et al., 2013, Nature Methods Vol. 10: 957-963).

The guide polynucleotide/Cas endonuclease system can be used in combination with at least one polynucleotide modification template to allow for editing (modification) of a genomic nucleotide sequence of interest. (See also US20150082478, published 19 Mar. 2015 and WO2015026886 published 26 Feb. 2015).

Polynucleotides of interest and/or traits can be stacked together in a complex trait locus as described in WO2012129373 published 27 Sep. 2012, and in WO2013112686, published 1 Aug. 2013. The guide polynucleotide/Cas endonuclease system described herein provides for an efficient system to generate double-strand breaks and allows for traits to be stacked in a complex trait locus.

In addition to the double-strand break inducing agents, site-specific base conversions can also be achieved to engineer one or more nucleotide changes to create one or more edits into the genome. These include for example, a site-specific base edit mediated by an C·G to T·A or an A·T to G·C base editing deaminase enzymes (Gaudelli et al., Programmable base editing of A·T to G·C in genomic DNA without DNA cleavage.” Nature (2017); Nishida et al. “Targeted nucleotide editing using hybrid prokaryotic and vertebrate adaptive immune systems.” Science 353 (6305) (2016); Komor et al. “Programmable editing of a target base in genomic DNA without double-stranded DNA cleavage.” Nature 533 (7603) (2016):420-4. A catalytically “dead” or inactive Cas9 (dCas9), for example a catalytically inactive “dead” version of a Cas9 ortholog disclosed herein, fused to a cytidine deaminase or an adenine deaminase protein becomes a specific base editor that can alter DNA bases without inducing a DNA break. Base editors convert C->T (or G->A on the opposite strand) or an adenine base editor that would convert adenine to inosine, resulting in an A->G change within an editing window specified by the gRNA.

### Introduction of Components

The methods and compositions described herein do not depend on a particular method for introducing a sequence into an organism or cell, only that the polynucleotide or polypeptide gains access to the interior of at least one cell of the organism. Introducing includes reference to the incorporation of a nucleic acid into a eukaryotic or prokaryotic cell where the nucleic acid may be incorporated into the genome of the cell, and includes reference to the transient (direct) provision of a nucleic acid, protein or polynucleotide-protein complex (PGEN, RGEN) to the cell. This does not include transient expression of genes from a T-strand where the gene is not integrated. In this case, the T-strand has moved from the Agrobacterium into the nucleus of a cell where only transient expression but not integration occurs.

Methods for introducing polynucleotides or polypeptides or a polynucleotide-protein complex into cells or organisms are known in the art including, but not limited to, microinjection, electroporation, stable transformation methods, transient transformation methods, ballistic particle acceleration (particle bombardment), whiskers mediated transformation, Agrobacterium-mediated transformation, direct gene transfer, viral-mediated introduction, transfection, transduction, cell-penetrating peptides, mesoporous silica nanoparticle (MSN)-mediated direct protein delivery, topical applications, sexual crossing, sexual breeding, and any combination thereof.

For example, the guide polynucleotide (guide RNA, crNucleotide+tracrNucleotide, guide DNA and/or guide RNA-DNA molecule) can be introduced into a cell directly (transiently) as a single stranded or double stranded polynucleotide molecule. The guide RNA (or crRNA+tracrRNA) can also be introduced into a cell indirectly by introducing a recombinant DNA molecule comprising a heterologous nucleic acid fragment encoding the guide RNA (or crRNA+tracrRNA), operably linked to a specific promoter that is capable of transcribing the guide RNA (crRNA+tracrRNA molecules) in said cell. The specific promoter can be, but is not limited to, a RNA polymerase III promoter, which allow for transcription of RNA with precisely defined, unmodified, 5′- and 3′-ends (Ma et al., 2014, Mol. Ther. Nucleic Acids 3:e161; DiCarlo et al., 2013, Nucleic Acids Res. 41: 4336-4343; WO2015026887, published 26 Feb. 2015). Any promoter capable of transcribing the guide RNA in a cell can be used and includes a heat shock/heat inducible promoter operably linked to a nucleotide sequence encoding the guide RNA.

The Cas endonuclease, such as the Cas endonuclease described herein, can be introduced into a cell by directly introducing the Cas polypeptide itself (referred to as direct delivery of Cas endonuclease), the mRNA encoding the Cas protein, and/or the guide polynucleotide/Cas endonuclease complex itself, using any method known in the art. The Cas endonuclease can also be introduced into a cell indirectly by introducing a recombinant DNA molecule that encodes the Cas endonuclease. The endonuclease can be introduced into a cell transiently or can be incorporated into the genome of the host cell using any method known in the art. Uptake of the endonuclease and/or the guided polynucleotide into the cell can be facilitated with a Cell Penetrating Peptide (CPP) as described in WO2016073433 published 12 May 2016. Any promoter capable of expressing the Cas endonuclease in a cell can be used and includes a heat shock/heat inducible promoter operably linked to a nucleotide sequence encoding the Cas endonuclease.

Direct delivery of a polynucleotide modification template into plant cells can be achieved through particle mediated delivery, and any other direct method of delivery, such as but not limiting to, polyethylene glycol (PEG)-mediated transfection to protoplasts, whiskers mediated transformation, electroporation, particle bombardment, cell-penetrating peptides, or mesoporous silica nanoparticle (MSN)-mediated direct protein delivery can be successfully used for delivering a polynucleotide modification template in eukaryotic cells, such as plant cells.

The donor DNA can be introduced by any means known in the art. The donor DNA may be provided by any transformation method known in the art including, for example, Agrobacterium-mediated transformation or biolistic particle bombardment. The donor DNA may be present transiently in the cell or it could be introduced via a viral replicon. In the presence of the Cas endonuclease and the target site, the donor DNA is inserted into the transformed plant's genome.

Direct delivery of any one of the guided Cas system components can be accompanied by direct delivery (co-delivery) of other mRNAs that can promote the enrichment and/or visualization of cells receiving the guide polynucleotide/Cas endonuclease complex components. For example, direct co-delivery of the guide polynucleotide/Cas endonuclease components (and/or guide polynucleotide/Cas endonuclease complex itself) together with mRNA encoding phenotypic markers (such as but not limiting to transcriptional activators such as CRC (Bruce et al. 2000 The Plant Cell 12:65-79) can enable the selection and enrichment of cells without the use of an exogenous selectable marker by restoring function to a non-functional gene product as described in WO2017070032 published 27 Apr. 2017.

The crc gene, when stably transformed into a cell, can render that cell unable to regenerate (confer anti-regeneration properties upon that cell). Transient transformation of a cell with crc would enable the cell to be selected, as it would serve as a marker for successful non-integration of the vector components included with the crc gene. Other examples of polynucleotides or genes that confer a useful phenotype (such as a stimulated growth or morphogenic response) during the period after transformation but before regeneration of a plant include but are not limited to WUS, BBM (or ODP2), BBM2, LEC2, KN1, IPT, MONOPTERUS-DELTA, LEC1, FUSCA, ABI3, SERK, and AGL15. Anti-regeneration genes can include genes from Agrobacterium that stimulate a growth response such as IPT, AV-6b, IAAm, and IAAh. Anti-regeneration genes can also include plant cell cycle genes such as CycA, CycB, CycD, and viral genes that simulate the cell cycle such as Wheat Dwarf Virus RepA, or the Malvastrum Yellow Vien Virus C4 gene. Generally, factors that impede normal regeneration and normal plant fertility can be used to provide the anti-regeneration property.

Introducing a guide RNA/Cas endonuclease complex described herein, into a cell includes introducing the individual components of said complex either separately or combined into the cell, and either directly (direct delivery as RNA for the guide and protein for the Cas endonuclease and Cas protein subunits, or functional fragments thereof) or via recombination constructs expressing the components (guide RNA, Cas endonuclease, Cas protein subunits, or functional fragments thereof). Introducing a guide RNA/Cas endonuclease complex (RGEN) into a cell includes introducing the guide RNA/Cas endonuclease complex as a ribonucleotide-protein into the cell. The ribonucleotide-protein can be assembled prior to being introduced into the cell as described herein. The components comprising the guide RNA/Cas endonuclease ribonucleotide protein (at least one Cas endonuclease, at least one guide RNA, at least one Cas protein subunits) can be assembled in vitro or assembled by any means known in the art prior to being introduced into a cell (targeted for genome modification as described herein).

Plant cells differ from human and animal cells in that plant cells comprise a plant cell wall which may act as a barrier to the direct delivery of the RGEN ribonucleoproteins and/or of the direct delivery of the RGEN components.

Direct delivery of the RGEN ribonucleoproteins into plant cells can be achieved through particle mediated delivery (particle bombardment. Based on the experiments described herein, a skilled artesian can now envision that any other direct method of delivery, such as but not limiting to, polyethylene glycol (PEG)-mediated transfection to protoplasts, electroporation, cell-penetrating peptides, or mesoporous silica nanoparticle (MSN)-mediated direct protein delivery, can be successfully used for delivering RGEN ribonucleoproteins into plant cells.

Direct delivery of the RGEN ribonucleoprotein, allows for genome editing at a target site in the genome of a cell which can be followed by rapid degradation of the complex, and only a transient presence of the complex in the cell. This transient presence of the RGEN complex may lead to reduced off-target effects. In contrast, delivery of RGEN components (guide RNA, Cas endonuclease) via plasmid DNA sequences can result in constant expression of RGENs from these plasmids which can intensify off target effects (Cradick, T. J. et al. (2013) Nucleic Acids Res 41:9584-9592; Fu, Y et al. (2014) Nat. Biotechnol. 31:822-826).

Direct delivery can be achieved by combining any one component of the guide RNA/Cas endonuclease complex (RGEN) (such as at least one guide RNA, at least one Cas protein, and at least one Cas protein), with a particle delivery matrix comprising a microparticle (such as but not limited to of a gold particle, tungsten particle, and silicon carbide whisker particle) (see also WO2017070032 published 27 Apr. 2017).

In one aspect, the guide polynucleotide/Cas endonuclease complex is a complex wherein the guide RNA and Cas endonuclease protein forming the guide RNA/Cas endonuclease complex are introduced into the cell as RNA and protein, respectively. In one aspect, the guide polynucleotide/Cas endonuclease complex is a complex wherein the guide RNA and Cas endonuclease protein and the at least one protein subunit of a Cas protein forming the guide RNA/Cas endonuclease complex are introduced into the cell as RNA and proteins, respectively. In one aspect, the guide polynucleotide/Cas endonuclease complex is a complex wherein the guide RNA and Cas endonuclease protein and the at least one protein subunit of a Cascade forming the guide RNA/Cas endonuclease complex (cleavage ready cascade) are preassembled in vitro and introduced into the cell as a ribonucleotide-protein complex.

Alternatively, polynucleotides may be introduced into plant or plant cells by contacting cells or organisms with a virus or viral nucleic acids. Generally, such methods involve incorporating a polynucleotide within a viral DNA or RNA molecule. In some examples a polypeptide of interest may be initially synthesized as part of a viral polyprotein, which is later processed by proteolysis in vivo or in vitro to produce the desired recombinant protein.

The polynucleotide or recombinant DNA construct can be provided to or introduced into a prokaryotic and eukaryotic cell or organism using a variety of transient transformation methods. Such transient transformation methods include, but are not limited to, the introduction of the polynucleotide construct directly into the plant.

Nucleic acids and proteins can be provided to a cell by any method including methods using molecules to facilitate the uptake of anyone or all components of a guided Cas system (protein and/or nucleic acids), such as cell-penetrating peptides and nanocarriers. See also US20110035836 published 10 Feb. 2011, and EP2821486A1 published 7 Jan. 2015.

Other methods of introducing polynucleotides into a prokaryotic and eukaryotic cell or organism or plant part can be used, including plastid transformation methods, and the methods for introducing polynucleotides into tissues from seedlings or mature seeds.

Stable transformation is intended to mean that the nucleotide construct introduced into an organism integrates into a genome of the organism and is capable of being inherited by the progeny thereof. Transient transformation is intended to mean that a polynucleotide is introduced into the organism and does not integrate into a genome of the organism or a polypeptide is introduced into an organism. Transient transformation indicates that the introduced composition is only temporarily expressed or present in the organism.

A variety of methods are available to identify those cells having an altered genome at or near a target site without using a screenable marker phenotype. Such methods can be viewed as directly analyzing a target sequence to detect any change in the target sequence, including but not limited to PCR methods, sequencing methods, nuclease digestion, Southern blots, and any combination thereof.

The presently disclosed polynucleotides and polypeptides can be introduced into a cell. Cells include, but are not limited to, human, non-human, animal, mammalian, bacterial, protist, fungal, insect, yeast, non-conventional yeast, and plant cells, as well as plants and seeds produced by the methods described herein. In some aspects, the cell of the organism is a reproductive cell, a somatic cell, a meiotic cell, a mitotic cell, a stem cell, or a pluripotent stem cell.

### Cells and Plants

The presently disclosed polynucleotides and polypeptides can be introduced into a plant cell. Plant cells include, well as plants and seeds produced by the methods described herein. Any plant can be used with the compositions and methods described herein, including monocot and dicot plants, and plant elements.

The compositions herein may be used to edit the genome of a plant cell in various ways. In one aspect, it may be desirable to delete one or more nucleotides. In another aspect, it may be desirable to insert one or more nucleotides. In one aspect, it may be desirable to replace one or more nucleotides. In another aspect, it may be desirable to modify one or more nucleotides via a covalent or non-covalent interaction with another atom or molecule. In some aspects, the cell is diploid. In some aspects, the cell is haploid.

Genome modification via a Cas endonuclease may be used to effect a genotypic and/or phenotypic change on the target organism. Such a change is preferably related to an improved trait of interest or an agronomically-important characteristic, the correction of an endogenous defect, or the expression of some type of expression marker. In some aspects, the trait of interest or agronomically-important characteristic is related to the overall health, fitness, or fertility of the plant, the yield of a plant product, the ecological fitness of the plant, or the environmental stability of the plant. In some aspects, the trait of interest or agronomically-important characteristic is selected from the group consisting of: agronomics, herbicide resistance, insecticide resistance, disease resistance, nematode resistance, microbial resistance, fungal resistance, viral resistance, fertility or sterility, grain characteristics, commercial product production. In some aspects, the trait of interest or agronomically-important characteristic is selected from the group consisting of: disease resistance, drought tolerance, heat tolerance, cold tolerance, salinity tolerance, metal tolerance, herbicide tolerance, improved water use efficiency, improved nitrogen utilization, improved nitrogen fixation, pest resistance, herbivore resistance, pathogen resistance, yield improvement, health enhancement, vigor improvement, growth improvement, photosynthetic capability improvement, nutrition enhancement, altered protein content, altered starch content, altered carbohydrate content, altered sugar content, altered fiber content, altered oil content, increased biomass, increased shoot length, increased root length, improved root architecture, modulation of a metabolite, modulation of the proteome, increased seed weight, altered seed carbohydrate composition, altered seed oil composition, altered seed protein composition, altered seed nutrient composition, as compared to an isoline plant not comprising a modification derived from the methods or compositions herein.

Examples of monocot plants that can be used include, but are not limited to, corn (Zea mays), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana), wheat (Triticum species, for example Triticum aestivum, Triticum monococcum), sugarcane (Saccharum spp.), oats (Avena), barley (Hordeum), switchgrass (Panicum virgatum), pineapple (Ananas comosus), banana (Musa spp.), palm, ornamentals, turfgrasses, and other grasses.

Examples of dicot plants that can be used include, but are not limited to, soybean (Glycine max), Brassica species (for example but not limited to: oilseed rape or Canola) (Brassica napus, B. campestris, Brassica rapa, Brassica juncea), alfalfa (Medicago sativa), tobacco (Nicotiana tabacum), Arabidopsis (Arabidopsis thaliana), sunflower (Helianthus annuus), cotton (Gossypium arboreum, Gossypium barbadense), peanut (Arachis hypogaea), tomato (Solanum lycopersicum), potato (Solanum tuberosum), various fruit trees, and cannabis.

Additional plants that can be used include safflower (Carthamus tinctorius), sweet potato (Ipomoea batatus), cassava (Manihot esculenta), coffee (Coffea spp.), coconut (Cocos nucifera), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Caricapapaya), cashew (Anacardium occidentale), macadamia (Macadamia integrifolia), almond (Prunus amygdalus), sugar beets (Beta vulgaris), vegetables, ornamentals, and conifers.

Vegetables that can be used include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be used include pines such as loblolly pine (Pinus taeda), slash pine (Pinus elliotii), ponderosa pine (Pinus ponderosa), lodgepole pine (Pinus contorta), and Monterey pine (Pinus radiata); Douglas fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow cedar (Chamaecyparis nootkatensis).

In certain embodiments of the disclosure, a fertile plant is a plant that produces viable male and female gametes and is self-fertile. Such a self-fertile plant can produce a progeny plant without the contribution from any other plant of a gamete and the genetic material comprised therein. Other embodiments of the disclosure can involve the use of a plant that is not self-fertile because the plant does not produce male gametes, or female gametes, or both, that are viable or otherwise capable of fertilization.

The present disclosure finds use in the breeding of plants comprising one or more introduced traits, or edited genomes.

A non-limiting example of how two traits can be stacked into the genome at a genetic distance of, for example, 5 cM from each other is described as follows: A first plant comprising a first transgenic target site integrated into a first DSB target site within the genomic window and not having the first genomic locus of interest is crossed to a second transgenic plant, comprising a genomic locus of interest at a different genomic insertion site within the genomic window and the second plant does not comprise the first transgenic target site. About 5% of the plant progeny from this cross will have both the first transgenic target site integrated into a first DSB target site and the first genomic locus of interest integrated at different genomic insertion sites within the genomic window. Progeny plants having both sites in the defined genomic window can be further crossed with a third transgenic plant comprising a second transgenic target site integrated into a second DSB target site and/or a second genomic locus of interest within the defined genomic window and lacking the first transgenic target site and the first genomic locus of interest. Progeny are then selected having the first transgenic target site, the first genomic locus of interest and the second genomic locus of interest integrated at different genomic insertion sites within the genomic window. Such methods can be used to produce a transgenic plant comprising a complex trait locus having at least 1, 2, 3, 4, 5, 6, 7, 8, 9, 10, 11, 12, 13, 14, 15, 16, 17, 19, 19, 20, 21, 22, 23, 24, 25, 26, 27, 28, 29, 30, 31 or more transgenic target sites integrated into DSB target sites and/or genomic loci of interest integrated at different sites within the genomic window. In such a manner, various complex trait loci can be generated.

While the invention has been particularly shown and described with reference to a preferred embodiment and various alternate embodiments, it will be understood by persons skilled in the relevant art that various changes in form and details can be made therein without departing from the spirit and scope of the invention. For instance, while the particular examples below may illustrate the methods and embodiments described herein using a specific plant, the principles in these examples may be applied to any plant. Therefore, it will be appreciated that the scope of this invention is encompassed by the embodiments of the inventions recited herein and in the specification rather than the specific examples that are exemplified below. All cited patents and publications referred to in this application are herein incorporated by reference in their entirety, for all purposes, to the same extent as if each were individually and specifically incorporated by reference.

### EXAMPLES

The following are examples of specific embodiments of some aspects of the invention. The examples are offered for illustrative purposes only, and are not intended to limit the scope of the invention in any way. Efforts have been made to ensure accuracy with respect to numbers used (e.g., amounts, temperatures, etc.), but some experimental error and deviation should, of course, be allowed for.

The meaning of abbreviations is as follows: “sec” means second(s), “min” means minute(s), “h” means hour(s), “d” means day(s), “μL” or “uL” or “μl” or “ul” means microliter(s), “mL” means milliliter(s), “L” means liter(s), “μM” means micromolar, “mM” means millimolar, “M” means molar, “mmol” means millimole(s), “μmole” or “umole” mean micromole(s), “g” means gram(s), “μg” or “ug” means microgram(s), “ng” means nanogram(s), “U” means unit(s), “bp” means base pair(s) and “kB” means kilobase(s).

### Example 1: Plasmids

Plasmids comprising T-DNA described in Table 1 were used in experiments described herein below.

TABLE 1
Plasmid Compositions:

| SEQ | | |
|---|---|---|
| ID | | |
| NO: | Plasmid ID | T-DNA comprises these components: |
| 1 | PHP21139 | ZM-IN2-2 PRO::ZM-WUS2::ZM-IN2-1 TERM |
| 2 | PHP22297 | FRT1:BAR + UBI PRO::CFP::PINII TERM + FRT87 |
| 3 | PHP5096 | UBI PRO::FLP::PINII TERM |
| 4 | PHP21875 | UBI PRO::ZM-ODP2::PINII |
| 5 | PHP89030 | PLTP PRO::ZM-ODP2::OS-T28 TERM |
| 6 | PHP89179 | PLTP PRO::ZM-WUS2::IN2-1 TERM |
| 7 | T-DNA from | RB + BUFFER1 + MINI-ALLSTOPS3 + PSA2 + LOXP + ATTB4 + |
| | RV022904 | ZM-SEQ11 (GENOMIC) (EDH5G) + UBI1ZM PRO::NPTII (MO1): |
| | | SB-UBI TERM + ZM-SEQ12 (GENOMIC) (EDH5G) + ATTB2 + |
| | | SB-UBI PRO + SB-UBI INTRON1::ZS-GREEN1: OS-UBI TERM + |
| | | MINI-ALLSTOPS + ATTB3 + LOXP + MINI-ALLSTOPS + PSB1 + |
| | | MINI-ALLSTOPS + MINI-ALLSTOPS4 + LB |
| 8 | T-DNA from | RB + BUFFER1 + PSE(3.1) + ATTB4 + FMV ENHANCER + PCSV |
| | RV022905 | ENH + MMV ENH + ZM-PLTP PRO::ZM-WUS2 (ALT1):IN2-1 |
| | | TERM + PSW1 + ATTB1 + UBI1ZM PRO::SV40 NLS: CAS9 |
| | | EXON1 (SP) (MO):ST-LS1 INTRON2: CAS9 EXON2 (SP) |
| | | (MO):VIRD2 NLS:SB-UBI TERM + PSN1 + ZM-U6 POLIII CHR8 |
| | | PRO: ZM CHR1-53.66-45CR1: GUIDE RNA: ZM-U6 POLIII CHR8 |
| | | TERM + PSN2 + ATTB2 + NOS PRO::CRC (ALT6):SB-GKAF |
| | | TERM (MOD1) |
| 9 | T-DNA from | RB + BUFFER1 + PSE (3.1) + ATTB4 + FMV ENHANCER + |
| | RV022901 | PCSV ENH + MMV ENH + ZM-PLTP PRO::ZM-WUS2 |
| | | (ALT1):IN2-1 TERM + PSW1 + ATTB1 + UBI1ZM PRO::SV40 |
| | | NLS: CAS9 EXON1 (SP) (MO):ST-LS1 INTRON2: CAS9 EXON2 |
| | | (SP) (MO):VIRD2 NLS:SB-UBI TERM + PSN1 + ATTB2 + NOS |
| | | PRO::CRC (ALT6): SB-GKAF TERM (MOD1) + ATTB3 + PSH + |
| | | LB |
| 10 | T-DNA from | RB + BUFFER1 + MINI-ALLSTOPS3 + PSA2 + LOXP + ATTB4 + |
| | RV022903 | ZM-U6 POLIII CHR8 PRO: ZM CHR1-53.66-45CR1 + GUIDE |
| | | RNA:ZM-U6 POLIII CHR8 TERM + PSN2 + ZM-SEQ11 |
| | | (GENOMIC) (EDH5G) + UBI1ZM PRO::NPTII (MO1): SB-UBI |
| | | TERM + ZM-SEQ12 (GENOMIC) (EDH5G) + ATTB2 + SB-UBI |
| | | PRO::ZS-GREEN1:OS-UBI TERM + MINI-ALLSTOPS + ATTB3 + |
| | | LOXP + MINI-ALLSTOPS + PSB1 + MINI-ALLSTOPS + MINI- |
| | | ALLSTOPS4 + LB |
| 11 | T-DNA from | RB + BUFFER1 + PSE (3.1) + ATTB4 + FMV ENHANCER + |
| | RV022900 | PCSV ENH + MMV ENH + ZM-PLTP PRO::ZM-WUS2 |
| | | (ALT1)(TR2)-V1:TAV-T2A:(MO1)SV40 NLS:CAS9 EXON1 (SP) |
| | | (MO):ST-LS1 INTRON2: CAS9 EXON2 (SP) (MO):VIRD2 |
| | | NLS:SB-UBI TERM + PSN1 + ATTB2 + NOS PRO::CRC |
| | | (ALT6):SB-GKAF TERM (MOD1) + ATTB3 + PSH + LB |
| 12 | T-DNA from | RB + BUFFER1 + MINI-ALLSTOPS3 + PSA2 + LOXP + ATTB4 + |
| | PHV00025 | ZM CHR1-53.66-45CR1 TARGET SITE + ZM-SEQ11 (GENOMIC) |
| | | (EDH5G) + UBI1ZM PRO::NPTII (MO1): SB-UBI TERM + ZM- |
| | | SEQ12 (GENOMIC) (EDH5G) + ZM CHR1-53.66-45CR1 TARGET |
| | | SITE + ATTB2 + SB-UBI PRO + SB-UBI INTRON1::ZS-GREEN1: |
| | | OS-UBI TERM + MINI-ALLSTOPS + ATTB3 + LOXP + MINI- |
| | | ALLSTOPS + PSB1 + MINI-ALLSTOPS + MINI-ALLSTOPS4 + |
| | | LB |
| 13 | Synthetic plasmid | REP A (AR) (ALT1) + REP B (AR) + REP C (AR) (MOD1) + SPC |
| | PHV00026 | (VER2) + NPTII + PUC ORI + nopalineRB + BUFFER1 + MINI- |
| | | ALLSTOPS3 + PSA2 + FMV ENHANCER + PCSV ENH + MMV |
| | | ENH + ZM-PLTP PRO::ZM-WUS2 (ALT1):IN2-1 TERM + PSW1 + |
| | | ATTB1 + UBI1ZM PRO::SV40 NLS: CAS9 EXON1 (SP) (MO):ST- |
| | | LS1 INTRON2: CAS9 EXON2 (SP) (MO):VIRD2 NLS:SB-UBI |
| | | TERM + PSN1 + ATTB2 + ZM-U6 POLIII CHR8 PRO: ZM CHR1- |
| | | 53.66-45CR1: GUIDE RNA: ZM-U6 POLIII CHR8 TERM + PSN2 + |
| | | NOS PRO::CRC (ALT6):SB-GKAF TERM (MOD1) + ATTB3 + |
| | | PSH + LB |
| 14 | Synthetic plasmid | GENTAMYCIN + PVS1 ORI + RB + BUFFER1 + MINI- |
| | PHV00027 | ALLSTOPS3 + PSA2 + ZM-SEQ11 (GENOMIC) (EDH5G) + |
| | | UBI1ZM PRO::NPTII (MO1): SB-UBI TERM + ZM-SEQ12 |
| | | (GENOMIC) (EDH5G) + ATTB2 + SB-UBI PRO::ZS-GREEN1:OS- |
| | | UBI TERM + MINI-ALLSTOPS + ATTB3 + LOXP + MINI- |
| | | ALLSTOPS + PSB1 + MINI-ALLSTOPS + MINI-ALLSTOPS4 + |
| | | LB |
| 15 | T-DNA from | RB + LOXP + NOS PRO::ZM-WUS2::IN2-1 TERM + FMV |
| | PHV00022 | ENHANCER::PCSV ENH::MMV ENH::UBI1ZM PRO::UBI1ZM |
| | | 5UTR::UBI1ZM INTRON1::ZM-ODP2::OS-T28 TERM + HSP17.7 |
| | | PRO::CRE EXON1::ST-LS1 INTRON2::CRE EXON2::PINII TERM + |
| | | LOXP + UBI1ZM PRO::UBI1ZM 5UTR::UBI1ZM |
| | | INTRON1::SV40 NLS:CAS9 EXON1 (MO)::ST-LSI |
| | | INTRON2::CAS9 EXON2 (MO):VIRD2 NLS::PINII TERM + |
| | | UBI1ZM PRO::UBI1ZM 5UTR::UBI1ZM INTRON1::NPTII::PINII |
| | | TERM + SB-UBI PRO::SB-UBI INTRON1::ZS-GREEN::OS-UBI |
| | | TERM + LB |
| 16 | T-DNA from | RB + LOXP + NOS PRO::ZM-WUS2::IN2-1 TERM + FMV |
| | PHV00023 | ENHANCER::PCSV ENH::MMV ENH::UBI1ZM PRO::UBI1ZM |
| | | 5UTR::UBI1ZM INTRON1::ZM-ODP2::OS-T28 TERM + HSP17.7 |
| | | PRO::CRE EXON1::ST-LS1 INTRON2::CRE EXON2::PINII TERM + |
| | | LOXP + UBI1ZM PRO::UBI1ZM 5UTR::UBI1ZM |
| | | INTRON1::SV40 NLS:CAS9 EXON1 (MO)::ST-LSI |
| | | INTRON2::CAS9 EXON2 (MO):VIRD2 NLS::PINII TERM + ZM- |
| | | U6 POLIII CHR8 PRO::ZM-ALS-CR7 GRNA::ZM-U6 POLIII |
| | | CHR8 TERM + ZM-U6 POLIII CHR8 PRO::ZM-WXY-CR4 |
| | | GRNA::ZM-U6 POLIII CHR8 TERM + ZM-U6 POLIII CHR8 |
| | | PRO::ZM-WXY-CR8 GRNA::ZM-U6 POLIII CHR8 TERM + SB- |
| | | UBI PRO::SB-UBI INTRON1::ZS-GREEN::OS-UBI TERM + LB |
| 17 | T-DNA from | RB + ZM-U6 POLIII CHR8 PRO::ZM-WXY-CR4 GRNA::ZM-U6 |
| | PHV00024 | POLIII CHR8 TERM + + ZM-U6 POLIII CHR8 PRO::ZM-WXY- |
| | | CR8 GRNA::ZM-U6 POLIII CHR8 TERM + ZM-U6 POLIII CHR8 |
| | | PRO::ZM-ALS-CR7 GRNA::ZM-U6 POLIII CHR8 TERM + |
| | | UBI1ZM PRO::UBI1ZM 5UTR::UBI1ZM INTRON1::DS- |
| | | RED2::PINII TERM + LB |
| 18 | CBF1A | Maize-optimized CBF1a activation domain coding sequence |
| 19 | CBF1A | Encoded CBF1A activation domain |
| 20 | PHP86489 | NOS PRO::CRC::SB-GKAF TERM |
| 21 | PHP665 synthetic | CAMV 35S ENH::CAMV 35S PRO::ADH INTRON1::C1::PINII |
| | construct | TERM |
| 22 | PV225 synthetic | UBI1ZM PRO::UBI1ZM 5UTR::UBI1ZM INTRON1::SV40 |
| | construct | NLS:CAS9 EXON1 (MO)::ST-LSI INTRON2::CAS9 EXON2 |
| | | (MO):VIRD2 NLS::PINII TERM |
| 23 | PV521 synthetic | ZM-U6 POLIII CHR8 PRO::ZM-R1 GRNA::ZM-U6 POLIII CHR8 |
| | construct | TERM |
| 24 | PV523 synthetic | ZM-U6 POLIII CHR8 PRO::ZM-R2 GRNA::ZM-U6 POLIII CHR8 |
| | construct | TERM |
| 25 | PV528 synthetic | ZM-U6 POLIII CHR8 PRO::ZM-R3 GRNA::ZM-U6 POLIII CHR8 |
| | construct | TERM |
| 26 | CAS-ALPHA10 | Synthetic sequence encoding the CAS-ALPHA10 (ALT23) |
| | (ALT23) | |
| 27 | dCAS-ALPHA10 | Encoded protein CAS-ALPHA10 (ALT23). |
| | (ALT23) | A40G + E81G + A87K + T335R Cas-alpha endonuclease. Optimal at |
| | | 37C. |
| 28 | dCAS-ALPHA10 | Synthetic sequence encoding the Dead-CAS-ALPHA10 (dALT23) |
| | (ALT23) | |
| 29 | dCAS-ALPHA10 | Encoded protein Dead-CAS-ALPHA10 (dALT23). |
| | (ALT23) | A40G + E81G + A87K + T335R + D228A dCas-alpha endonuclease. |
| | | Optimal at 37C. |
| 30 | RV045092 | ZM1UBI PRO::dCAS-ALPHA10:CBF1a::UBI TERM |
| 31 | RV045093 | ZM1UBI PRO::dCAS-ALPHA10:CBF1a::UBI TERM |
| 32 | RV045094 | ZM1UBI PRO::dCAS-ALPHA10:CBF1a::UBI TERM |
| 33 | RV045095 | ZM1UBI PRO::dCAS-ALPHA10:CBF1a::UBI TERM |
| 34 | RV045096 | ZM1UBI PRO::dCAS-ALPHA10:CBF1a::UBI TERM |
| 35 | RV045097 | ZM1UBI PRO::dCAS-ALPHA10:CBF1a::UBI TERM |
| 36 | PHV00012 | U6 PRO::gRNAr1::U6 TERM |
| 37 | PHV00013 | U6 PRO::gRNAr2::U6 TERM |
| 38 | PHV00014 | U6 PRO::gRNAr3::U6 TERM |
| 39 | Cas-alpha10 variant | Engineered variant of Cas-alpha10 |
| 40 | CAS-ALPHA10 | Synthetic sequence encoding the CAS-ALPHA10 (ALT35) |
| | (ALT35) | |
| 41 | CAS-ALPHA10 | Encoded protein CAS-ALPHA10 (ALT35). |
| | (ALT35) | A40G + E81G + A87K + T335R + T190K + T217H + K298S + H306F + |
| | | I405N Cas-alpha endonuclease. Optimal at 30C and above. |
| 42 | PHV00015 | RB + 3xENH::PLTP PRO::WUS2 + NOS::CRC + UBI1ZM |
| | | PRO::KN1CPP: PROTEIN LINKER1: CAS-ALPHA10 (ALT 35) + |
| | | U6 PRO: gRNA-w1 + U6 PRO:gRNA-w2 + LB |
| 43 | Protein Linker1 | Nucleic acid sequence encoding Protein Linker1 |
| 44 | KN1CPP | Nucleic acid sequence encoding Knotted1 Cell Penetrating Peptide |
| 45 | CAS-ALPHA10 | Synthetic sequence CAS-ALPHA10 (ALT 30C-1) |
| | (ALT 30C-1) | |
| 46 | CAS-ALPHA10 | Synthetic sequence CAS-ALPHA10 (ALT 30C-2) |
| | (ALT 30C-2) | |
| 47 | CAS-ALPHA10 | Synthetic sequence CAS-ALPHA10 (ALT 30C-3) |
| | (ALT 30C-3) | |
| 48 | CAS-ALPHA10 | Synthetic sequence CAS-ALPHA10 (ALT 30C-4) |
| | (ALT 30C-4) | |
| 49 | CAS-ALPHA10 | Synthetic sequence CAS-ALPHA10 (ALT 30C-5) |
| | (ALT 30C-5) | |
| 50 | CAS-ALPHA10 | Synthetic sequence CAS-ALPHA10 (ALT 30C-6) |
| | (ALT 30C-6) | |
| 51 | CAS-ALPHA10 | Synthetic sequence CAS-ALPHA10 (ALT 30C-7) |
| | (ALT 30C-7) | |
| 52 | CAS-ALPHA10 | Synthetic sequence CAS-ALPHA10 (ALT 30C-8) |
| | (ALT 30C-8) | |
| 53 | CAS-ALPHA10 | Synthetic sequence CAS-ALPHA10 (ALT 30C-9) |
| | (ALT 30C-9) | |
| 54 | PHV00016 | RB + 3xENH::PLTP PRO::WUS2 + NOS::CRC + UBI1ZM |
| | | PRO::KN1CPP:PROTEIN LINKER1:CAS-ALPHA10 (ALT 4) + U6 |
| | | PRO: gRNA-w1 + U6 PRO:gRNA-w2 + NOS::CRC + LB |
| 55 | PHV00017 | RB + Hv-LTP2 PRO::CRC + UBI1ZM PRO::CAS-ALPHA10 (ALT |
| | | 4) + LB |
| 56 | PHV00018 | RB + 3xENH::PLTP PRO::WUS2 + U6 PRO: gRNA-w1 + U6 |
| | | PRO:gRNA-w2 + Si-UBI PRO::ZS-GREEN + LB |
| 57 | PHV00019 | RB + HSP17.7 PRO::WUS2 + UBI1ZM PRO::CAS-ALPHA10 (ALT |
| | | 4) + LTP2::CRC + LB |
| 58 | PHV00020 | RB + U6 PRO::gRNA-53.66 + CHR1-53.66 TARGET SITE::SEQ |
| | | 11-HR1:SI-UBI PRO::NPTII::SI_UBI TERM:SEQ12-HR2::CHR1- |
| | | 53.66 TARGET SITE + SB-UBI PRO::ZS-GREEN::SB-UBI TERM + |
| | | LB |
| 59 | CAS-ALPHA10 | Synthetic sequence encoding the CAS-ALPHA10 (ALT4) Exon2 |
| | EXON1 (MO1) | |
| 60 | CAS-ALPHA10 | Synthetic sequence encoding the CAS-ALPHA10 (ALT4) Exon2 |
| | EXON2 (MO1) | |
| | (TR1) (ALT4) | |
| 61 | CAS-ALPHA10 | Encoded protein CAS-ALPHA10 (ALT4). A40G + E81G + T335R Cas- |
| | (ALT4) | alpha endonuclease. Optimal at 37C |
| 62 | GRMZM2G028622- | WUS2 Genomic sequence from B73 |
| | WUS2genomic | |
| 63 | gRNA-a10-wus2-1 | gRNA1 for endogenous WUS2 promoter |
| 64 | gRNA-a10-wus2-2 | gRNA2 for endogenous WUS2 promoter |
| 65 | gRNA-a10-wus2-3 | gRNA3 for endogenous WUS2 promoter |
| 66 | gRNA-a10-wus2-4 | gRNA4 for endogenous WUS2 promoter |
| 67 | gRNA-a10-wus2-5 | gRNA5 for endogenous WUS2 promoter |
| 68 | gRNA-a10-wus2-6 | gRNA6 for endogenous WUS2 promoter |
| 69 | PVH00021 | RB + UBI1ZM PRO::dCAS9-ALPHA10:CBF1A::PINII TERM + U6 |
| | | PRO:gRNA-wus1 + U6 PRO:gRNA-wus2 + U6 PRO:gRNA-wus3 + + |
| | | U6 PRO:gRNA-bbm1 + U6 PRO:gRNA-bbm2 + U6 PRO:gRNA- |
| | | bbm3 + SB-UBI PRO::ZS-GREEN::OS-UBI TERM + LB |
| 70 | gRNA-a10-odp2-1 | gRNA1 for endogenous ODP2 promoter |
| 71 | gRNA-a10-odp2-2 | gRNA2 for endogenous ODP2 promoter |
| 72 | gRNA-a10-odp2-3 | gRNA3 for endogenous ODP2 promoter |
| 73 | gRNA-a10-odp2-4 | gRNA4 for endogenous ODP2 promoter |
| 74 | gRNA-a10-odp2-5 | gRNA5 for endogenous ODP2 promoter |
| 75 | gRNA-a10-odp2-6 | gRNA6 for endogenous ODP2 promoter |

### Example 2: Culture Media

Various media are referenced in the Examples for use in transformation and cell culture. These media are described below in Tables 2-9.

TABLE 2
Sorghum Transformation Media Compositions

PHI-I: 4.3 g/l MS salts (Phytotechnology Laboratories, Shawnee Mission, KS,
catalog number M524), 0.5 mg/l nicotinic acid, 0.5 mg/l pyridoxine HCl, 1 mg/l
thiamine HC1, 0.1 g/l myo-inositol, 1 g/l casamino acids (Becton Dickinson and
Company, BD Diagnostic Systems, Sparks, MD, catalog number 223050), 1.5 mg/l
2,4-dichlorophenoxyacetic acid (2,4-D), 68.5 g/l sucrose, 36 g/l glucose, pH 5.2;
with 100 μM acetosyringone added before using.
PHI-T: PHI-I with 20 g/l sucrose, 10 g/l glucose, 2 mg/l 2,4-D, no casamino acids,
0.5 g/l MES buffer, 0.7 g/l L-proline, 10 mg/l ascorbic acid, 100 μM
acetosyringone, 8 g/l agar, pH 5.8.
PHI-U: PHI-T with 1.5 mg/1 2,4-D 100 mg/l carbenicillin, 30 g/l sucrose, no glucose
and acetosyringone; 5 mg/l PPT(GLUFOSINATE-NH4), pH 5.8.
PHI-UM: PHI-U with12.5 g/l mannose and 5 g/l maltose, no sucrose, no
PPT(GLUFOSINATE-NH4), pH 5.8
PHI-V: PHI-U with 10 mg/l PPT(GLUFOSINATE-NH4)
DBC3: 4.3 g/l MS salts, 0.25 g/l myo-inositol, 1.0 g/l casein hydrolysate, 1.0 mg/l
thiamine HCL, 1.0 mg/l 2,4-D, 30 g/l maltose, 0.69 g/l L-proline, 1.22 mg/l cupric
sulfate, 0.5 mg/l BAP, 3.5 g/l phytagel, pH 5.8
PHI-X: 4.3 g/l MS salts, 0.1 g/l myo-inositol, 5.0 ml MS vitamins stockª, 0.5 mg/l
zeatin, 700 mg/l L-proline, 60 g/l sucrose, 1 mg/l indole-3-acetic acid, 0.1 μM
abscisic acid, 0.1 mg/l thidiazuron, 100 mg/l carbenicillin, 5 mg/l
PPT(GLUFOSINATE-NH4), 8 g/l agar, pH 5.6.
PHI-XM: PHI-X with no PPT(GLUFOSINATE-NH4); added 1.25 mg/l cupric
sulfate, pH 5.6.
PHI-Z: 2.15 g/l MS salts, 0.05 g/l myo-inositol, 2.5 ml MS vitamins stockª, 20 g/l
sucrose, 3 g/l phytagel, pH 5.6
^{a}MS vitamins stock: 0.1 g/l nicotinic acid, 0.1 g/l pyridoxine HCl, 0.02 g/l thiamine HCl, 0.4 g/l glycine

TABLE 3
WI 4
WI 4

| DI water | 1000 | mL |
|---|---|---|
| MS salt + Vitamins(M519) | 4.43 | g |
| Maltose | 30 | g |
| Glucose | 10 | g |
| MES | 1.95 | g |
| 2,4-D (.5 mg/L) | 1 | ml |
| Picloram (10 mg/ml) | 200 | μl |
| BAP (1 mg/L) | .5 | ml |
| Adjust PH to 5.8 with KOH | | |
| Post sterilization add: | | |
| Acetosyringone (400 μM) | 400 | μl |

TABLE 4
WC # 10
WC # 10

| DI water | 1000 | mL |
|---|---|---|
| MS salt + Vitamins(M519) | 4.43 | g |
| Maltose | 30 | g |
| Glucose | 1 | g |
| MES | 1.95 | g |
| 2,4-D (.5 mg/L) | 1 | ml |
| Picloram (10 mg/ml) | 200 | μl |
| BAP (1 mg/L) | .5 | ml |
| 50X CuSO4 (.1M) | 49 | μl |
| Adjust PH to 5.8 with KOH and add 2.5 g/L | | |
| of Phytagel. | | |
| Post sterilization add: | | |
| Acetosyringone (400 μM) | 400 | μl |

TABLE 5
DBC4
DBC4

| dd H20 | 1000 | mL |
|---|---|---|
| MS salt | 4.3 | g |
| Maltose | 30 | g |
| Myo-inositol | 0.25 | g |
| N-Z-Amine-A | 1 | g |
| Proline | 0.69 | g |
| Thiamine-HCl (0.1 mg/mL) | 10 | mL |
| 50X CuSO4 (0.1M) | 49 | μL |
| 2,4-D (0.5 mg/mL) | 2 | mL |
| BAP | 1 | mL |
| Adjust PH to 5.8 with KOH and then add | | |
| 3.5 g/L of Phytagel. | | |
| Post sterilization add: | | |
| Cef(100 mg/ml) | 1 | ml |

TABLE 6
DBC6
DBC6

| dd H20 | 1000 | mL |
|---|---|---|
| MS salt | 4.3 | g |
| Maltose | 30 | g |
| Myo-inositol | 0.25 | g |
| N-Z-Amine-A | 1 | g |
| Proline | 0.69 | g |
| Thiamine-HCl (0.1 mg/mL) | 10 | mL |
| 50X CuSO4 (0.1M) | 49 | μL |
| 2,4-D (0.5 mg/mL) | 1 | mL |
| BAP | 2 | mL |
| Adjust PH to 5.8 with KOH and then add | | |
| 3.5 g/L of Phytagel. | | |
| Post sterilization add: | | |
| Cef(100 mg/ml) | 1 | ml |

TABLE 7
MSA
MSA

| dd H20 | 1000 | mL |
|---|---|---|
| MS salt + Vitamins(M519) | 4.43 | g |
| Sucorse | 20 | g |
| Myo-Inositol | 1 | g |
| Adjust PH to 5.8 with KOH and then add | | |
| 3.5 g/L of Phytagel. | | |
| Post steriliaztion add: | | |
| Cef(100 mg/ml) | 1 | ml |

TABLE 8
MSB
MSB

| dd H20 | 1000 | mL |
|---|---|---|
| MS salt + Vitamins(M519) | 4.43 | g |
| Sucorse | 20 | g |
| Myo-Inositol | 1 | g |
| Adjust PH to 5.8 with KOH and then add | | |
| 3.5 g/L of Phytagel. | | |
| Post steriliaztion add: | | |
| Cef(100 mg/ml) | 1 | ml |
| IBA | .5 | ml |

TABLE 9
Media Compositions-

| | Units | | | | | | | |
|---|---|---|---|---|---|---|---|---|
| Medium components | per liter | 12V | 810I | 700 | 710I | 605J | 605T | 289Q |
| MS BASAL SALT | g | | | 4.3 | 4.3 | 4.3 | 4.3 | 4.3 |
| MIXTURE | | | | | | | | |
| N6 MACRONUTRIENTS | ml | | | | | 60.0 | 60.0 | |
| 10X | | | | | | | | |
| POTASSIUM NITRATE | g | | | | | 1.7 | 1.7 | |
| B5H MINOR SALTS | ml | | | | | 0.6 | 0.6 | |
| 1000X | | | | | | | | |
| NaFe EDTA FOR B5H | ml | | | | | 6.0 | 6.0 | |
| 100X | | | | | | | | |
| ERIKSSON'S VITAMINS | ml | | | | | 0.4 | 0.4 | |
| 1000X | | | | | | | | |
| S&H VITAMIN STOCK | ml | | | | | 6.0 | 6.0 | |
| 100X | | | | | | | | |
| THIAMINE .HCL | mg | | | 10.0 | 10.0 | 0.5 | 0.5 | |
| L-PROLINE | g | | | | 0.7 | 2.0 | 2.0 | 0.7 |
| CASEIN | g | | | | | 0.3 | 0.3 | |
| HYDROLYSATE (ACID) | | | | | | | | |
| SUCROSE | g | | | 68.5 | 20.0 | 20.0 | 20.0 | 60.0 |
| GLUCOSE | g | 5.0 | | 36.0 | 10.0 | 0.6 | 0.6 | |
| MALTOSE | g | | | | | | | |
| 2,4-D | mg | | | 1.5 | 2.0 | 0.8 | 0.8 | |
| AGAR | g | 15.0 | 15.0 | | 8.0 | 6.0 | 6.0 | 8.0 |
| PHYTAGEL | g | | | | | | | |
| DICAMBA | g | | | | | 1.2 | 1.2 | |
| SILVER NITRATE | mg | | | | | 3.4 | 3.4 | |
| AGRIBIO Carbenicillin | mg | | | | | 100 | | |
| Timentin | mg | | | | | | 150 | 150 |
| Cefotaxime | mg | | | | | | 100 | 100 |
| MYO-INOSITOL | g | | | 0.1 | 0.1 | | | 0.1 |
| NICOTINIC ACID | mg | | | 0.5 | 0.5 | | | |
| PYRIDOXINE.HCL | mg | | | 0.5 | 0.5 | | | |
| VITAMIN ASSAY | g | | | 1.0 | | | | |
| CASAMINO ACIDS | | | | | | | | |
| MES BUFFER | g | | | | 0.5 | | | |
| ACETOSYRINGONE | uM | | | | 100 | | | |
| ASCORBIC ACID | mg | | | | 10.0 | | | |
| 10 MG/ML (7S) | | | | | | | | |
| MS VITAMIN STOCK | ml | | | | | | | 5.0 |
| SOL. | | | | | | | | |
| ZEATIN | mg | | | | | | | 0.5 |
| CUPRIC SULFATE | mg | | | | | | | 1.3 |
| IAA 0.5 MG/ML (28A) | ml | | | | | | | 2.0 |
| ABA 0.1 mm | ml | | | | | | | 1.0 |
| THIDIAZURON | mg | | | | | | | 0.1 |
| AGRIBIO Carbenicillin | mg | | | | | | | 100.0 |
| PPT(GLUFOSINATE- | mg | | | | | | | |
| NH4) | | | | | | | | |
| BAP | mg | | | | | | | 1.0 |
| YEAST EXTRACT (BD | g | | 5.0 | | | | | |
| Difco) | | | | | | | | |
| PEPTONE | g | | 10.0 | | | | | |
| SODIUM CHLORIDE | g | | 5.0 | | | | | |
| SPECTINOMYCIN | mg | 50.0 | 100 | | | | | |
| FERROUS | ml | 2.0 | | | | | | |
| SULFATE.7H20 | | | | | | | | |
| AB BUFFER 20X (12D) | ml | 50.0 | | | | | | |
| AB SALTS 20X (12E) | ml | 50.0 | | | | | | |
| Benomyl | mg | | | | | | | |
| pH | | | | | | | | 5.6 |
| | Units | | | | | | | |
| Medium components | per liter | 289R | 13158H | 13224B | 13266K | 272X | 272V | 13158 |
| MS BASAL SALT | g | 4.3 | 4.3 | | 4.3 | 4.3 | 4.3 | 4.3 |
| MIXTURE | | | | | | | | |
| N6 MACRONUTRIENTS | ml | | | 4.0 | 60.0 | | | |
| 10X | | | | | | | | |
| POTASSIUM NITRATE | g | | | | 1.7 | | | |
| B5H MINOR SALTS | ml | | | | 0.6 | | | |
| 1000X | | | | | | | | |
| NaFe EDTA FOR B5H | ml | | | | 6.0 | | | |
| 100X | | | | | | | | |
| ERIKSSON'S VITAMINS | ml | | | 1.0 | 0.4 | | | |
| 1000X | | | | | | | | |
| S&H VITAMIN STOCK | ml | | | | 6.0 | | | |
| 100X | | | | | | | | |
| THIAMINE .HCL | mg | | | 0.5 | 0.5 | | | |
| L-PROLINE | g | 0.7 | 0.7 | 2.9 | 2.0 | | | |
| CASEIN | g | | | | 0.3 | | | |
| HYDROLYSATE (ACID) | | | | | | | | |
| SUCROSE | g | 60.0 | 60.0 | 190.0 | 20.0 | 40.0 | 40.0 | 40.0 |
| GLUCOSE | g | | | | 0.6 | | | |
| MALTOSE | g | | | | | | | |
| 2,4-D | mg | | | | 1.6 | | | |
| AGAR | g | | 8.0 | 6.4 | 6.0 | 6.0 | 6.0 | 6.0 |
| PHYTAGEL | g | | | | | | | |
| DICAMBA | g | | | | 1.2 | | | |
| SILVER NITRATE | mg | | | 8.5 | 1.7 | | | |
| AGRIBIO Carbenicillin | mg | | | | 2.0 | | | |
| Timentin | mg | 150 | 150 | | | | | |
| Cefotaxime | mg | 100 | 100 | 25 | 25 | | | |
| MYO-INOSITOL | g | 0.1 | 0.1 | | | 0.1 | 0.1 | 0.1 |
| NICOTINIC ACID | mg | | | | | | | |
| PYRIDOXINE.HCL | mg | | | | | | | |
| VITAMIN ASSAY | g | | | | | | | |
| CASAMINO ACIDS | | | | | | | | |
| MES BUFFER | g | | | | | | | |
| ACETOSYRINGONE | uM | | | | | | | |
| ASCORBIC ACID | mg | | | | | | | |
| 10MG/ML (7S) | | | | | | | | |
| MS VITAMIN STOCK | ml | 5.0 | 5.0 | | | 5.0 | 5.0 | 5.0 |
| SOL. | | | | | | | | |
| ZEATIN | mg | 0.5 | 0.5 | | | | | |
| CUPRIC SULFATE | mg | 1.3 | 1.3 | | | | | |
| IAA 0.5 MG/ML (28A) | ml | 2.0 | 2.0 | | | | | |
| ABA 0.1 mm | ml | 1.0 | 1.0 | | | | | |
| THIDIAZURON | mg | 0.1 | 0.1 | | | | | |
| AGRIBIO Carbenicillin | mg | | | | | | | |
| PPT(GLUFOSINATE- | mg | | | | | | | |
| NH4) | | | | | | | | |
| BAP | mg | | | | | | | |
| YEAST EXTRACT (BD | g | | | | | | | |
| Difco) | | | | | | | | |
| PEPTONE | g | | | | | | | |
| SODIUM CHLORIDE | g | | | | | | | |
| SPECTINOMYCIN | mg | | | | | | | |
| FERROUS | ml | | | | | | | |
| SULFATE.7H20 | | | | | | | | |
| AB BUFFER 20X (12D) | ml | | | | | | | |
| AB SALTS 20X (12E) | ml | | | | | | | |
| Benomyl | mg | | | | | | | 100.0 |
| pH | | | | | | 0.5 | 5.6 | |

### Example 3: Particle Bombardment

Standard protocols for particle bombardment (Finer and McMullen, 1991, In Vitro Cell Dev. Biol.—Plant 27:175-182) can be used with the methods of the disclosure.

A. Particle-Mediated Delivery for Cas9-Mediated Donor Template Integration Via Homology-Dependent Repair (Hdr)

Four plasmids were typically used for each particle bombardment; 1) the donor plasmid (50 ng/μl) containing the donor cassette flanked by homology-arms (genomic sequence) for CRISPR/Cas9-mediated homology-dependent SDN3, 2) a plasmid (50 ng/μl) containing the expression cassette UBI PRO::Cas9::pinII plus an expression cassette ZM-U6 PRO::gRNA::U6 TERM, 3) a plasmid (10 ng/μl) containing the expression cassette 3×ENH::UBI PRO::ODP2, and 4) a plasmid (5 ng/μl) containing the expression cassette NOS::WUS2::IN2 TERM. To attach the DNA to 0.6 μm gold particles, the four plasmids were mixed by adding 10 μl of each plasmid together in a low-binding microfuge tube (Sorenson Bioscience 39640T) for a total of 40 μl. To this suspension, 50 μl of 0.6 μm gold particles (30 μg/μl) and 1.0 μl of Transit 20/20 (Cat No MIR5404, Mirus Bio LLC) were added, and the suspension was placed on a rotary shaker for 10 minutes. The suspension was centrifuged at 10,000 RPM (˜9400×g) and the supernatant was discarded. The gold particles were re-suspended in 120 μl of 100% ethanol, briefly sonicated at low power and 10 μl was pipetted onto each carrier disc. The carrier discs were then air-dried to evaporate away all the remaining ethanol. Particle bombardment was performed using a PDF-1000/IE Particle Delivery Device, at 27 inches Hg using a 600 PSI rupture disc.

A transgenic Pioneer Stiff-Stalk inbred PHH5E was used in this experiment. Hemizygous seed was selected based on seed-specific expression of AM-CYAN1 and was surface sterilized using 80% ethanol for 3 minutes, followed by incubation in a solution of 50% bleach+0.1% Tween-20 while agitating with a stir-bar for 20 minutes. The sterile seed were then rinsed 3 times in sterile double-distilled water. Surface-sterilized seed were germinated on 13158F solid medium under (120 μE m-2 s-1) lights using an 18-hour photoperiod at 25° C.

Alternatively, chlorine gas or oxidizing agents can be used for seed sterilization. Chlorine gas can be generated using a variety of compounds (or agents), including bleaching powders, calcium hypochlorite, sodium hypochlorite, industrial bleach, household bleach, chlorine dioxide monochloramine, dichloramine, and trichloramine. Oxidizing agents that can be used in the method include but are not limited to, ozone, hydrogen peroxide, hypochlorous acid, hypobromous acid, chlorine dioxide, and ethylene dioxide.

After 14 days, the 3 cm segment directly above the seedling mesocotyl was excised (containing the leaf-whorl tissue directly above the apical meristem region of the stem). The 3 cm segment was bisected longitudinally using a scalpel. Then the outer layer of leaf tissue (coleoptile) was discarded. For the leaf tissue derived from each seedling, the leaves were separated and laid flat within a 2 cm diameter in the middle of a culture plate containing one of the two following media; i) medium 13224 containing 12% sucrose for 3-4 hr before bombardment (10 plates, each containing tissue from one of 10 seedlings and, ii) medium 13224C containing 12% sucrose+0.1 mg/l ethametsulfuron for 2-3 hours before bombardment (10 plates, each containing tissue from one of 10 seedlings).

Plasmids PHP21875 (SEQ ID NO: 4), PHP21139 (SEQ ID NO: 1), PHP71193 (SEQ ID NO: 76), and PHP71788 (SEQ ID NO: 77) were used. Preparation of DNA-functionalized gold particles was done as follows. Stock solutions of plasmids PHP71193 and PHP71788 (100 ng/μl) were diluted to 50 ng/μl with sterile water. Stock solutions of PHP21875 and PHP21139 (100 ng/μl) were diluted to 25 ng/μl with sterile water. Using sterile, low-binding Eppendorf tubes. Ten ul each of the diluted plasmids PHP71788 (50 ng/μl), PHP71193 (50 ng/μl), PHP21875 (25 ng/μl), and PHP21139 (25 ng/μl), were added to a sterile, low-binding Eppendorf tube (final ratio of plasmids was 50:50:25:25, respectively). This DNA mixture was then added to a sterile-low-binding Eppendorf tube containing 50 ul of 0.6 uM gold particles at a stock concentration of 10 mg/ml) and gently agitated to mix the DNA and gold in the suspension. One ul of Transit 20/20 was added and the tube again gently agitating. The tube was then placed on a 125 RPM rotator shaker for 10 minutes at room temperature. The tube was then centrifuged at 10,000 RPM in a microfuge. The supernatant was discarded and after adding 120 ul of 95% EtOH, the tube was sonicated briefly on a low setting to resuspend the particles and then 10 ul of the DNA/gold/EtOH suspension was pipetted onto the center of the carrier disc. The carrier discs were left exposed to the sterile air low in the laminar flow hood for approximately 10 minutes to evaporate the EtOH. The carrier discs with dried gold/DNA were then used for particle bombardment. For particle bombardment, a PDS-1000/He Particle Delivery System (Bio-rad, Hercules, CA, USA) was used, with 425 psi rupture disc, and the petri dish containing the target tissue positioned two shelves below the carrier-holder, and a vacuum of approximately 27 mg Hg.

When expression of Wus2 and Odp2 was induced by addition of ethametsulfuron, somatic embryogenesis was stimulated in leaf tissue. Using this inducible Wus2/Odp2 germplasm as the starting point for a new experiment, seedling-derived leaf tissue was then used as the target explant for particle bombardment. As mentioned above, in one treatment the leaf tissue was incubated on culture medium with 12% sucrose (to plasmolyze the leaf cells) prior to particle bombardment, and in the second treatment the leaf segments were exposed to culture medium with 12% sucrose plus 0.1 mg/l ethametsulfuron prior to particle delivery (providing an earlier exposure to the inductive treatment to begin stimulation of Wus2/Odp2 expression). To further enhance morphogenesis (beyond that provided by inducible expression), plasmids containing constitutive Wus2 and ODP2 expression cassettes were co-delivered with Cas9 and gRNA, as well as the template DNA (the genomic-sequence-flanked NPTII expression cassette). After DNA delivery, successful NPTII coding sequence integration via homology-dependent recombination (HDR) permitted regeneration of HDR events using both the inducing ligand (0.1 mg/l ethametsulfuron) and G418 for selection. Due to high levels of Wus2 and Bbm expression (inducible-expression from pre-integrated 60850-T-DNA plus constitutive expression provided by PHP21875 and PHP21139), selection using NPTII and G418 became less efficient, resulting in escape (wild type) plants being recovered. Thus, at lower levels of G418 selective agent (150 or 200 mg/l), when leaf tissue from 9 seedlings was used as starting explants for each treatment, 46 and 34 TO plants containing the NPTII gene were recovered but none were observed to contain perfect HDR integrations. In contrast, when 9 seedlings were again used for particle delivery of the plasmids followed by increased selective pressure due to higher G418 (250 mg/l), selection became more stringent and three perfect HDR integration events were recovered from a total of 38 TO plants that were regenerated and analyzed.

Thus, using this combination of Wus2 and Odp2 expression cassettes to stimulate growth while also delivering the SDN3 donor DNA, the Cas9 expression cassette, and the guide-RNA expression cassette resulted in efficient homology-dependent targeted integration. Thus, three perfect HDR events were recovered from particle bombardment of leaf segments derived from only 34 starting seedlings.

In comparison, when wild-type maize Stiff-Stalk inbred PHH5G was transformed in a similar manner but without the use of Wus2 and Odp2, transgenic events were not recovered. Thus, particle delivery of the plasmids PHP71193 and PHP71788 into seedling-derived leaf tissue (with no Wus2 or Odp2) does not result in transgenic or edited TO plants.

B. Site-Specific Integration

Pioneer inbred PH184C (disclosed in U.S. Pat. No. 8,445,763, incorporated herein by reference in its entirety) that contains in chromosome-1 a pre-integrated Site-Specific Integration (SSI) target site (Chrom-1 target site) composed of UBI PRO:FRT1:NPTII::PINII TERM+FRT87 is used. Prior to bombardment, 10-12 DAP (days after pollination) immature embryos are isolated from ears of Pioneer inbred PH184C and placed on 605J culture medium plus 16% sucrose for three hours to plasmolyze the scutellar cells. Alternatively, the first 2-3 cm of seedling-derived leaf-whorl tissue is bisected longitudinally and sliced into approximately 0.5-3.0 mm leaf segments, and these leaf segments are plasmolyzed on 605J medium plus 16% sucrose for three hours prior to particle bombardment.

Four plasmids are typically used for each particle bombardment:
1) a donor plasmid (100 ng/μl) containing a FRT-flanked donor cassette for Recombinase-Mediated Cassette Exchange, for example a plasmid containing FRT1:PMI:: PINII TERM::CZ19B1 TERM+UBI1ZM PRO::UBI1ZM 5 UTR::UBI1ZM INTRON1::DS-RED2::PINII TERM+FRT6 (PHP8418-0004; SEQ ID NO: 78);
2) a plasmid (2.5 ng/μl) containing the expression cassette UBI1ZM PRO::UBI1ZM 5 UTR::UBI1ZM INTRON1::FLPm::PINII TERM (PHP5096);
3) a plasmid (10 ng/μl) containing the expression cassette ZM-PLTP PRO::ZM-ODP2::OS-T28 TERM+FMV & PCSV ENHANCERS (PHP89030); and
4) a plasmid (5 ng/μl) containing the expression cassette ZM-PLTP PRO::ZM-WUS2::IN2-1 TERM+PSW1+GZ-W64A TERM+FL2 TERM (PHP89179).

To attach the DNA to 0.6 μm gold particles, the four plasmids are mixed by adding 10 μl of each plasmid together in a low-binding microfuge tube (Sorenson Bioscience 39640T) for a total of 40 μl. To this suspension, 50 μl of 0.6 μm gold particles (30 μg/μl) and 1.0 μl of Transit 20/20 (Cat No MIR5404, Mirus Bio LLC) are added, and the suspension is placed on a rotary shaker for 10 minutes. The suspension is centrifuged at 10,000 RPM (˜9400×g) and the supernatant is discarded. The gold particles are re-suspended in 120 μl of 100% ethanol, briefly sonicated at low power and 10 μl is pipetted onto each carrier disc. The carrier discs are then air-dried to remove all remaining ethanol. Particle bombardment is performed using a Biolistics PDF-1000, at 28 inches of Mercury using a 200 PSI rupture disc. After particle bombardment, the immature embryos or leaf segments are selected on 605J medium modified to contain 12.5 g/l mannose and 5 g/l maltose and no sucrose. After 10-12 weeks on selection, plantlets are regenerated and analyzed using qPCR. It is expected that co-delivery of PLTP::ODP2 (PHP89030) and PLTP::WUS2 (PHP89179) along with the SSI components (Donor DNA (PHP8418-0004)+UBI::FLP (PHP5096)) will produce high frequencies of site-specific integration of the donor fragment into the Chrom-1 target site (i.e. at rates of 4-7% relative to the number of bombarded immature embryos).

### Example 4: Agrobacterium-Mediated Transformation of Corn

A. Preparation of Agrobacterium Master Plate.

Agrobacterium tumefaciens harboring a binary donor vector was streaked out from a −80° C. frozen aliquot onto solid 12R medium and cultured at 28° C. in the dark for 2-3 days to make a master plate.

B. Growing Agrobacterium on Solid Medium.

A single colony or multiple colonies ofAgrobacterium were picked from the master plate and streaked onto a second plate containing 810K medium and incubated at 28° C. in the dark overnight.

Agrobacterium infection medium (700A; 5 ml) and 100 mM 3′-5′-Dimethoxy-4′-hydroxyacetophenone (acetosyringone; 5 μL) were added to a 14 mL conical tube in a hood. About 3 full loops of Agrobacterium from the second plate were suspended in the tube and the tube was then vortexed to make an even suspension. The suspension (1 ml) was transferred to a spectrophotometer tube and the optical density (550 nm) of the suspension was adjusted to a reading of about 0.35-1.0. The Agrobacterium concentration was approximately 0.5 to 2.0×10⁹ cfu/mL. The final Agrobacterium suspension was aliquoted into 2 mL microcentrifuge tubes, each containing about 1 mL of the suspension. The suspensions were then used as soon as possible.

C. Growing Agrobacterium on Liquid Medium.

Alternatively, Agrobacterium can be prepared for transformation by growing in liquid medium. One day before infection, a 125 ml flask was prepared with 30 ml of 557A medium (10.5 g/l potassium phosphate dibasic, 4.5 g/l potassium phosphate monobasic anhydrous, 1 g/l ammonium sulfate, 0.5 g/l sodium citrate dehydrate, 10 g/l sucrose, 1 mM magnesium sulfate) and 30 μL spectinomycin (50 mg/mL) and 30 μL acetosyringone (20 mg/mL). A half loopful of Agrobacterium from a second plate was suspended into the flasks and placed on an orbital shaker set at 200 rpm and incubated at 28° C. overnight. The Agrobacterium culture was centrifuged at 5000 rpm for 10 min. The supernatant was removed and the Agrobacterium infection medium (700A) with acetosyringone solution was added. The bacteria were resuspended by vortex and the optical density (550 nm) of the Agrobacterium suspension was adjusted to a reading of about 0.35 to 2.0.

D. Maize Transformation.

1. Immature Embryo Transformation

Immature ears of a maize (Zea mays L.) cultivar were surface-sterilized for 15-20 min in 20% (v/v) bleach (5.25% sodium hypochlorite) plus 1 drop of Tween-20 followed by 3 washes in sterile water. Immature embryos (IEs) were isolated from ears and were placed in 2 ml of the Agrobacterium infection medium with acetosyringone solution. The optimal size of the embryos varies based on the inbred, but for transformation with WUS2 and ODP2 a wide size range of immature embryo sizes could be used. The solution was drawn off and 1 ml of Agrobacterium suspension was added to the embryos and the tube vortexed for 5-10 sec. The microfuge tube was allowed to stand for 5 min in the hood. The suspension of Agrobacterium and embryos were poured onto 7101 co-cultivation medium (see Table 9). Any embryos left in the tube were transferred to the plate using a sterile spatula. The Agrobacterium suspension was drawn off and the embryos placed axis side down on the media. The plate was sealed with Parafilm M® film (moisture resistant flexible plastic, available at Bemis Company, Inc., 1 Neenah Center 4th floor, PO Box 669, Neenah, WI 54957) and incubated in the dark at 21° C. for 1-3 days of co-cultivation.

Embryos were transferred to resting medium (605T medium) without selection. Three to 7 days later, they were transferred to maturation medium (289Q medium) supplemented with a selective agent.

2. Seedling-Derived Leaf Tissue Transformation

Mature maize seed was surface sterilized for 15-20 min in 20% (v/v) bleach (5.25% sodium hypochlorite) plus 1 drop of Tween 20 followed by 3 washes in sterile water, germinated and allowed to grow into seedlings for approximately 14 days, and then prepared to produce leaf fragments as described above. Leaf segments were placed in the Agrobacterium infection medium (700A) with 200 μM acetosyringone solution+0.02% Break-Thru® surfactant (Plant Health Technologies, P.O. Box 70013, Boise, ID 83707-0113). The Agrobacterium infection medium was drawn off and 1 ml of the Agrobacterium suspension was added to the leaf segments and was allowed to stand for 20 min. The suspension of Agrobacterium and leaf segments were poured through a sterile metal sieve and the liquid was discarded. The leaf segments collected on the metal sieve were transferred using a spatula onto a stack of 3 sterile Whatman #2 filter papers, used to wick off excess Agrobacterium-containing liquid, and then again a spatula was used to transfer the leaf segments onto a filter paper lying on co-cultivation medium. The plate was incubated in the dark at 21° C. for 1-3 days of co-cultivation.

The filter papers supporting the leaf segments were then transferred to resting medium (605T medium) without selection. Seven days later, the filter papers supporting the leaf segments were transferred to selection medium for three weeks. After selection, healthy growing somatic embryos were transferred using forceps onto maturation medium for two weeks in the dark, at which point the maturation plates were transferred in toto (still containing the maturing somatic embryos) into the light for an addition week. After one week in the light, regenerating plantlets were transferred to rooting medium. After rooting, plantlets were ready for transplanting to the greenhouse.

3. Transformation of Seedling-Derived Leaf Tissue in Maize Inbred PH85E with WUS2 and ODP2

Constitutive expression of WUS2 and ODP2 after Agrobacterium-mediated transformation of maize leaf segments resulted in production of embryogenic callus and/or rapidly formed somatic embryos which regenerate into healthy, fertile TO plants. The general protocol for Agrobacterium-mediated maize transformation described in Example above was used, with further detail on modifications below for using leaf tissue as the target explant.

In Vitro Seed Germination to Produce Seedling Target Tissue

Mature seeds of inbred PH85E were surface sterilized by immersion in a series of solutions under agitation using a magnetic stir bar; first in an 80% ethanol solution for 3 minutes, the ethanol solution was decanted and replaced with a 30% Clorox bleach solution containing 0.1% Tween-20 for 20 minutes, the Clorox bleach solution was decanted, and the mature seeds were rinsed (three 5-minute rinses) in autoclaved sterile water. The sterilized seeds were transferred onto solid 900 medium after the final sterile water rinse. In vitro germination and seedling growth were carried out at 26° C. with a 16 h light/8 h dark photoperiod. The first 2.5 to 3 cm of leaf whorl above the mesocotyl was removed from each 12-18 day-old seedling for further processing for transformation.

Alternatively, seeds may be sterilized by exposure to chlorine gas. Chlorine gas can be generated using a variety of compounds (or agents), including bleaching powders, calcium hypochlorite, sodium hypochlorite, industrial bleach, household bleach, chlorine dioxide monochloramine, dichloramine, and trichloramine. In addition, oxidizing agents can be used for seed sterilization. Oxidizing agents that can be used in the methods disclosed herein include but are not limited to, ozone, hydrogen peroxide, hypochlorous acid, hypobromous acid, chlorine dioxide, and ethylene dioxide.

Agrobacterium Preparation

Agrobacterium tumefaciens strain LBA4404 TD THY-harboring helper plasmid PHP71539 (SEQ ID NO: 4) (pVIR9, see US20190078106A1, herein incorporated by reference in its entirety) and a binary donor vector, PHP96037, containing a WUS2/ODP2 T-DNA with a selectable marker (ZM-ALS (HRA)) and a screenable marker (ZS-GREEN1) or a binary donor control vector containing a selectable marker and/or a screenable marker T-DNA (lacking WUS2/ODP2) was streaked out from a −80° C. frozen aliquot onto solid 12V medium and cultured at 28° C. in the dark for 2 days to make a master plate. A working plate was prepared by streaking 4-5 colonies from the 12V-grown master plate across fresh 810K media, incubating overnight in the dark at 28° C. prior to using for Agrobacterium infection. Additional helper plasmids (PHP70298, RV005393, and RV007497 (containing vir genes from A. rhizogenes)) useful in the methods of the disclosure are listed in Table 2.

Agrobacterium infection medium (700J medium, 10 ml) with the addition of 20 μL of acetosyringone and 20 μL of a previously 10-fold-diluted surfactant (Break Thru S 233, Evonik Industries GmbH, GoldschmidtstraBe 100, 45127 Essen, Germany) was added to a 50 mL conical tube in a hood. About 5 full loops of Agrobacterium were collected from the working plate, transferred to the infection medium in the 50 ml tube, and then vortexed until uniformly suspended. The suspension (1 ml) was transferred to a spectrophotometer tube and the optical density (550 nm) of the suspension was adjusted to a reading of 0.6. The final Agrobacterium suspension was aliquoted into Corning six-well plates containing 0.4 μm permeable culture inserts (Falcon, Part Numbers 353046 and 353090, respectively) with each well containing about 8 mL of the Agrobacterium suspension.

Seed of maize inbred PH85E were surface sterilized as previously described, and then germinated at 28° C. under low light on solid 90B medium (½ strength MS salts plus 20 g/l sucrose and 50 mg/l benomyl). The leaf base segment (an approximate 2.5-3.0 cm section above the mesocotyl) was removed from each 12-18 day-old in vitro-germinated seedling with sterilized scissors. These leaf segments were placed into a 150 mm×15 mm Petri dish. Forceps were used to hold each leaf whorl section at the upper green end and the section was bisected longitudinally into 2 lengthwise halves using a sterile #10 scalpel blade. The outer leaf was removed and the inner leaves of the whorl were then cross-cut (diced) into smaller sections (approximately 1 to 3 mm in size, preferably 2.5-3.0 mm in size). Small leaf sections were collected and directly transferred into the permeable culture inserts containing the Agrobacterium suspension and incubated at room temperature (25° C.) for a 15-minute infection period.

After infection, the culture insert containing the Agrobacterium-infected leaf segments was removed from the 8-well plate and placed on an autoclaved dry filter paper to wick up and remove any residual Agrobacterium solution. The infected leaf segments were then transferred onto a fresh filter paper (VWR 7.5 CM) resting on 710N solid co-cultivation medium. Forceps were used to evenly disperse the leaf segments on the 710N plates and to ensure they have enough room to grow. The infected leaf tissue was incubated at 21° C. in the dark for 2-3 days.

After 2-3d co-cultivation, the paper supporting the leaf tissue was removed from the 710N medium and transferred onto 605B medium for 4 week resting culture. Tissue was sub-cultured every 2 weeks. After the 4 weeks culture on resting medium (605B) the plates were placed into a controlled temperature/humidity incubator (45° C./70% RH) for a 2-hour heat treatment. The plates were removed from the incubator and kept at room temperature (25° C.) for 1-2 hours until the plates had cooled down. Depending on the maize inbred, a single two-hour heat treatment, or two 2-hour heat treatments on two consecutive days, were applied to stimulate the drought-inducible RAB17 promoter and induce CRE-mediated excision of WUS2, ODP2, and CRE recombinase.

After the heat treatment and temperature equilibration at room temperature, leaf segments with newly-developed somatic embryos were transferred onto 13329B maturation medium without filter papers, cultured in the dark at 28° C. for 2 weeks, and then moved into the a 26° C. light room for an additional week. Leaf segments that now supported small shoots were transferred onto 404J rooting medium for an additional 2-3 weeks until well formed roots had developed, at which point the plantlets were ready for transfer to the greenhouse.

Results from five experiments are shown in Table 15, in which 10 starting seedlings per experiment (50 total) were used to produce the starting leaf segments for Agrobacterium infection, the number of transgenic TO plants recovered ranged from 18 (Exp. 1) to 51 (Exp. 4), resulting in a mean transformation frequency of 360%+/−112 (Standard Deviation (SD)). This is in contrast to experiments in which only a selectable marker gene and/or a screenable marker gene (fluorescent protein gene) were contained in the T-DNA, in which no culture response was observed and no TO plants were produced.

In addition to a high transformation frequency, a high percentage of the recovered TO plants were single-copy (SC) for the T-DNA (containing the selectable marker and/or the screenable marker) with no contaminating sequences from Agrobacterium being detected. Such SC/No-Agro events (TO plants) ranged from 23% to 37% with a mean of 31.4% (+/−5.2% SD). By comparing the number of high-quality transgenic TO plants (SC for the T-DNA with no contaminating Agrobacterium backbone sequences) to the number of starting seedlings used in these experiments provided a clear measure of overall efficiency, with a mean frequency of 114% (+/−44% SD). This method using WUS2/ODP2 obviated the need for growing mature maize plants for 90-120 days in the greenhouse to produce immature embryo explants for transformation and provided transgenic events from leaf explants generated from germinated seed in the lab.

TABLE 15

| | | # of T0 | | # of SC | SC No- | SC No- | |
|---|---|---|---|---|---|---|---|
| | # of | plants | | No-Agro | Agro | Agro | Escape |
| Exp | Seedlings | (events) | T0 % | Seq | %* | %** | Freq. |
| 1 | 10 | 18 | 180% | 5 | 28% | 50% | 39% |
| 2 | 10 | 41 | 410% | 14 | 34% | 140% | 32% |
| 3 | 10 | 40 | 400% | 9 | 23% | 90% | 45% |
| 4 | 10 | 51 | 510% | 18 | 35% | 180% | 40% |
| 5 | 10 | 30 | 300% | 11 | 37% | 110% | 43% |
| | 50 | 180 | 360% | 57 | 29% | 114% | 39% |

*Frequency of T0 plants with single-copy T-DNA and no plasmid sequences, relative to the total number of T0 plants
**Frequency of T0 plants with single-copy T-DNA and no plasmid sequences, relative to the starting number of seedlings

### Example 5: Transformation of Sorghum Leaf Segments

Constitutive expression of WUS2 and ODP2 after Agrobacterium-mediated transformation of sorghum leaf segments results in production of embryogenic callus and/or rapidly formed somatic embryos which regenerate into healthy, fertile TO plants.

Agrobacterium strain, constructs, growth of seedlings, preparation of leaf material for transformation, Agrobacterium infection, co-culture, resting culture, maturation and rooting for sorghum were all the same as the methods developed for maize in Example 4. The purpose here was to determine how transferable the method was without any sorghum-specific optimization.

Results from four experiments using a WUS2/ODP2 T-DNA, along with one experiment in which the control T-DNA contained only a selectable marker and a fluorescent marker (HRA+ZS-GREEN) are shown in Table 16. Each experiment also contained a comparison between two resting media, 13266P (605B medium plus 50 mg/l meropenem) which contained no additional cupric sulfate or BAP and medium 13265L (13266P medium plus 100 uM cupric sulfate and 0.5 mg/l BAP).

As demonstrated for maize, sorghum treatments that contained WUS2 and ODP2 expression cassettes in the T-DNA (PHP96037) also resulted in high transformation frequencies, calculated based on the number oftransgenic TO plants recovered per starting seedling, with a mean (+/−SD) for 13266P and 13265L of 36.5 (+/−4.103) and 35.5% (+/−9.6) respectively, with no significant difference between the two media compositions (p=0.05). In contrast, the control treatment containing the selectable marker and/or the screenable marker with no WUS2/ODP2 in the T-DNA produced no transgenic events. The mean frequency of obtaining high-quality TO sorghum plants (single copy with no Agrobacterium backbone (SC/NA 0%)) when transformed with P1P96037 was between 367 to 38% for the two media.

As with maize, this method obviated the need for growing mature sorghum plants for 90-120 days in the greenhouse to produce immature embryos explants for transformation and provided transgenic events from leaf explants generated from germinated seed in the lab.

TABLE 16

| | | | | | # | | | |
|---|---|---|---|---|---|---|---|---|
| | | | | | SC/No | SC/ | | |
| | # | Treatment | # T0 | | Agro | NA | SC | # |
| Expt. No. | Seedlings | (Medium) | Plants | T0 % | (NA) | % | % | Escapes |
| 1 | 15 | 13265L | 65 | 433% | 26 | 40% | 30% | 2 |
| | 13 | 13266P | 18 | 138% | 7 | 39% | 7% | 2 |
| 2 | 15 | 13265L | 56 | 373% | 18 | 32% | 28% | 2 |
| | 15 | 13266P | 46 | 307% | 19 | 41% | 28% | 5 |
| 3 | 15 | 13265L | 32 | 213% | 7 | 22% | 7% | 0 |
| | 15 | 13266P | 14 | 93% | 5 | 36% | 13% | 0 |
| 4 | 16 | 13265L | 27 | 169% | 13 | 48% | 13% | 9 |
| | 16 | 13266P | 20 | 125% | 6 | 30% | 6% | 3 |
| Total | 61 | 13265L | 180 | 295% | 64 | 36% | 78% | 13 |
| Total | 59 | 13266P | 97 | 164% | 37 | 38% | 54% | 10 |
| No | 15 | 13265L | 0 | 0% | 0 | 0% | 0% | 0 |
| WUS/ODP2 | 15 | 13266P | 0 | 0% | 0 | 0% | 0% | 0 |
| Control | | | | | | | | |

### Example 6: Transformation of Cells with Two Rhizobiales Strains, One Comprising a Vector with an Integratable Polynucleotide and the Other Comprising a Vector with a Cas Endonuclease, Guide RNA, a Morphogenic Factor, and an Anti-Regeneration Gene

RV022904 (SEQ ID NO. 7) contained a donor sequence of HR arms (Homologous Recombination arms) flanking a UBIpro:NPTII, followed by a Ubipro:ZS-GREEN. RV022905 (SEQ ID NO. 8) contained triple enhancer PLTPpro:Wuschel, Ubipro:Cas9, a ZM U6 PolIII pro: gRNA cutting at TS45 in maize inbred ED85E, and a Nospro:CRC. Nospro:CRC inhibited regeneration if stably integrated by producing anthocyanin. Two strains of Agrobacterium tumefaciens, each harboring a binary donor RV022904 or RV022905 were struck out from a −80° C. frozen aliquot onto separate plates containing 810I medium and incubated for 1-2 days at 28° C. A single colony from each was picked and streaked out onto separate working plates and allowed to grow overnight at 28° C. The next morning in a sterile hood, the bacteria were individually scraped off and re-suspended in separate tubes of 14 ml infection media (700 medium; 5 ml) and 100 mM 3′-5′-Dimethoxy-4′-hydroxyacetophenone (acetosyringone; 5 μL) conical tube with a final OD of 0.1-0.2 at a wavelength of 550 nM. The Agrobacterium concentration was approximately 0.5 to 2.0×109 cfu/mL The Agrobacterium LBA4404 suspensions containing RV022904 and RV022905 were mixed in a 1:1 or 1:9 ratios with final volumes of 1 ml. In both cases, Agrobacterium containing RV022904 were the equal or larger volume. The suspensions were then used as soon as possible.

Ears of a maize (Zea mays L.) cultivar picked 11 days after pollination were surface-sterilized for 15-20 min in 20% (v/v) bleach (5.25% sodium hypochlorite) plus 1 drop of Tween 20 followed by 3 washes in sterile water. Immature embryos were isolated from ears and are placed in 2 ml of the infection medium with acetosyringone solution. Supernatant was removed and 1 ml of the Agro suspension was added to each tube of embryos. 500 embryos were transformed with the 1:1 ratio mix, and 500 embryos were transformed with the 1:9 ratio mix. A negative control 1:1 mix of RV022904 and RV022901 (SEQ ID NO. 9) (similar to RV022905 except not containing any guide RNA) was used to transform 200 embryos. After five minutes, embryos were removed, placed on solid co-cultivation medium plates, and oriented embryo axis down. Embryos were incubated at 21° C. in the dark for 1 day.

Embryos were then transferred to resting media and stored at 28C in the dark for 5-7 days. Tissues were then transferred to selection in the dark for 11-14 days.

At 7-12 days after Agrobacterium infection fluorescent multicellular structures were observed which continued to regenerate and produce plants. In contrast, multicellular structures containing anthocyanin did not regenerate.

Plantlets were moved to rooting with no selection for 7-14 days at 28° C. in the light. Leaves from individual plants were sampled for qRTPCR to confirm presence or absence of WUS and Cas9, and integration of UBIpro:NPTII at the SDN3 site. 27 Plantlets derived from the negative control Agro mix with no gRNA were sampled and tested for SDN3 frequency. None of the negative controls showed integration of UBIpro:NPTII at the cut site. 655 plantlets derived from the 1:1 Agrobacterium mix of RV022904 and RV022905 were sampled, of those 98 were escapes (negative for NPTII), 17 had low DNA, and 3 were PCR positive for both HR arms at the genomic cut site, demonstrating homology-dependent recombination (HDR). The overall HDR frequency of events was 0.54 percent. Of the positive SDN3 samples, all were PCR negative for presence WUS or Cas9 integration, indicating that the WUS and cas9 had acted transiently without integration.

About 456 plantlets derived from the 1:9 Agrobacterium mix of RV022904 and RV022905 were sampled. Of those, 147 were escapes and 24 had less DNA due to extraction differences. None of the samples yielded positive PCR for both HR arms at the genomic cut site, indicating no SDN3.

### Example 7: Transformation of Cells with Two Rhizobiales Strains, One Comprising a Vector with an Integratable Polynucleotide and a Guide RNA, and the Other Comprising a Vector with a Cas Endonuclease, Morphogenic Factor, and Anti-Regeneration Gene

RV022903 (SEQ ID NO. 10) contained a donor sequence of ZM U6 PolIII pro: gRNA cutting at TS45 in ED85E, HR arms flanking a UBIpro:NPTII, followed by a Ubipro:ZS-GREEN. RV022901 contained triple enhancer PLTPpro:Wuschel, Ubipro:Cas9, and a Nospro:CRC. Nospro:CRC inhibited regeneration if stably integrated by producing anthocyanin. As the guide RNA and Cas9 were split, cutting would only occur if both Agrobacteria transformed the same cell or if the guide RNA and/or Cas nuclease moves between cells (i.e., to another cell that was not directly transformed by the transformation process described herein to introduce the Cas enzyme, gRNA and/or the morphogenic factors). Two strains of Agrobacterium tumefaciens, each harboring a binary donor RV022901 or RV022903 were struck out from a −80° C. frozen aliquot onto separate plates containing 810I medium and incubated for 1-2 days at 28° C. A single colony from each was picked and streaked out onto separate working plates and allowed to grow overnight at 28° C. The next morning in a sterile hood, the bacteria were individually scraped off and re-suspended in separate tubes of 14 ml infection media (700 medium; 5 ml) and 100 mM 3′-5′-Dimethoxy-4′-hydroxyacetophenone (acetosyringone; 5 μL) conical tube with a final OD of 0.1-0.2 at a wavelength of 550 nM. The Agrobacterium concentration was approximately 0.5 to 2.0×10⁹ cfu/mL. The Agrobacterium LBA4404 suspensions containing RV022903 and RV022901 were mixed in a 1:1 or 1:9 ratios with final volumes of 1 ml. In both cases, Agrobacterium containing RV022903 were the equal or larger volume. The suspensions were then used as soon as possible.

Ears of a maize (Zea mays L.) cultivar picked 11 days after pollination were surface-sterilized for 15-20 min in 20% (v/v) bleach (5.25% sodium hypochlorite) plus 1 drop of Tween 20 followed by 3 washes in sterile water. Immature embryos were isolated from ears and are placed in 2 ml of the infection medium with acetosyringone solution. Supernatant was removed and 1 ml of the Agro suspension was added to each tube of embryos. 500 embryos were transformed with the 1:1 ratio mix, and 500 embryos were transformed with the 1:9 ratio mix. A negative control Agro mix not containing any guide RNA was used to transform 200 embryos. After five minutes, embryos were then removed and placed on solid co-cultivation medium plates and oriented embryo axis down. Embryos were incubated at 21° C. in the dark for 1 day.

Embryos were then transferred to resting media and stored at 28C in the dark for 5-7 days. Tissue were then transferred to selection in the dark for 11-14 days.

At 7-12 days after Agrobacterium infection fluorescent multicellular structures were observed which continued to develop and produce plants. In contrast, multicellular structures containing anthocyanin did not regenerate.

Plantlets were moved to rooting with no selection for 7-14 days at 28C in the light. Leafs from individual plants were sampled for qRTPCR to confirm presence or absence of WUS and Cas9, and integration of UBIpro:NPTII at the SDN3 site. 27 Plantlets derived from the negative control Agro mix with no gRNA were sampled and tested for SDN3 frequency. None of the negative controls showed integration of UBIpro:NPTII at the cut site. About 663 plantlets derived from the 1:1 Agrobacterium mix of RV022903 and RV022901 were sampled, of those 174 were escapes (negative for NPTII), 9 had low DNA, and 0 were PCR positive for both HR arms at the genomic cut site. About 472 plantlets derived from the 1:9 Agrobacterium mix of RV022903 and RV022901 were sampled, of those 147 were escapes (negative for NPTII), 8 had low DNA, and 2 were PCR positive for both HR arms at the genomic cut site, demonstrating homology-dependent recombination (HDR). The overall HDR frequency of events was 0.59 percent. Of the positive SDN3 samples, all were PCR negative for presence WUS or Cas9 integration, indicating that the WUS and Cas9 had acted transiently without integration.

### Example 8: Cutting Frequencies at 6 Days Post-Transformation

RV022903 contained a donor sequence of ZM U6 PolIII pro:gRNA cutting at TS45 in ED85E, HR arms flanking a UBIpro:NPTII, followed by a Ubipro:ZS-GREEN. RV022900 (SEQ ID NO. 11) contained triple enhancer PLTPpro:Wuschel:T2A:Cas9 (WUS and Cas9 linked by T2A (Liu et al. Sci Rep. 2017; 7(1):2193), and a Nospro:CRC. Nospro:CRC inhibited regeneration if stably integrated by producing anthocyanin. As the guide RNA and Cas9 were split, cutting would only occur if both agrobacteria transformed the same cell. Two strains of Agrobacterium tumefaciens, each harboring a binary donor RV022900 or RV022903 were struck out from a −80° C. frozen aliquot onto separate plates containing 810I medium and incubated for 1-2 days at 28° C. A single colony from each was picked and streaked out onto separate working plates and allowed to grow overnight at 28° C. The next morning in a sterile hood, the bacteria were individually scraped off and re-suspended in separate conical tubes of 14 ml infection media (700 medium; 5 ml) and 100 mM 3′-5′-Dimethoxy-4′-hydroxyacetophenone (acetosyringone; 5 μL) with a final OD of 0.4 at a wavelength of 550 nM. The Agrobacterium LBA4404 suspensions containing RV022903 and RV022900 were mixed in a 1:1 or 1:9 ratios with final volumes of lml. In both cases, Agrobacterium containing RV022903 were the equal or larger volume. The suspensions were then used as soon as possible.

Ears of a maize (Zea mays L.) cultivar picked 11 days after pollination were surface-sterilized for 15-20 min in 20% (v/v) bleach (5.25% sodium hypochlorite) plus 1 drop of Tween 20 followed by 3 washes in sterile water. Immature embryos were isolated from ears and are placed in 2 ml of the infection medium with acetosyringone solution. Supernatant was removed and 1 ml of the Agro suspension was added to each tube of embryos. 50 embryos were transformed with the 1:1 ratio mix, and 50 embryos were transformed with the 1:9 ratio mix. A negative control Agro mix not containing any guide RNA was used to transform 50 embryos. After five minutes, embryos were then removed and placed on solid co-cultivation medium plates and oriented embryo axis down. Embryos were incubated at 21° C. in the dark for 1 day.

Embryos were then transferred to resting media and stored at 28C in the dark for 5-7 days.

The above steps were also repeated for Agrobacterium containing RV022901 with RV022903, and RV022904 with RV022905. RV022905 alone was used as a positive control.

Immature embryos were collected and placed 20 per tube, frozen and lyophilized. After DNA extraction, cutting frequency was determined by PCR and sequencing over the TS45 cut site. Cut frequency was calculated as the number of sequence reads indicating a SNP or other polymorphism at TS45 divided by the total number of sequence reads. The average cut frequency for 40 embryos transformed with RV022900 with RV022903 at a 1:1 ratio was 0.62 percent, and 0.073 percent when transformed at a 1:9 ratio. The average cut frequency for 40 embryos transformed with RV022901 with RV022903 at a 1:1 ratio was 0.18, and 0.12 when transformed at a 1:9 ratio. RV022901 alone had a cut frequency of 0.01 percent. Positive control RV022905 alone had a cut frequency of 0.67 percent. The average cut frequency for 40 embryos transformed with RV022904 with RV022905 at a 1:1 ratio was 0.83 percent, and 0.28 percent when transformed at a 1:9 ratio.

TABLE 10
Cutting frequencies with different ratios of plasmids

| | Ratio/ | | Mutation |
|---|---|---|---|
| Replicate | Plasmid | Description | Frequency |
| A | 1:1 | RV022900:RV022903 | 0.03 |
| B | 1:1 00:03 | RV022900 & | 1.21 |
| | | RV022903 | |
| C | 1:9 00:03 | RV022900 & | 0.07 |
| | | RV022903 | |
| D | 1:9 00:03 | RV022900 & | 0.08 |
| | | RV022903 | |
| E | 01 Alone | neg control | 0.01 |
| F | 1:1 01:03 | RV022901 & | 0.18 |
| | | RV022903 | |
| G | 1:9 01:03 | RV022901 & | 0.12 |
| | | RV022903 | |
| H | 1:1 02:03 | RV022902 & | 0.00 |
| | | RV022903 | |
| I | 1:1 02:03 | RV022902 & | 0.02 |
| | | RV022903 | |
| J | 1:9 02:03 | RV022902 & | 0.00 |
| | | RV022903 | |
| K | 1:9 02:03 | RV022902 & | 0.00 |
| | | RV022903 | |
| L | 05 alone | positive control | 0.67 |
| | | RV022905 | |
| M | 1:1 05:04 | RV022905 & | 0.83 |
| | | RV022904 | |
| N | 1:9 05:04 | RV022905 & | 0.28 |
| | | RV022904 | |

### Example 9: Flanking Sites Added Outside of Hr Arms to Increase Sdn3 Frequency

P1TP34567 (SEQ ID NO. 12) contains a donor sequence of guide RNA cut sites outside of HR arms flanking a UBJpro:NPTII, followed by a Ubipro: Z S-GREEN. PLASMID E (RV022905) contains triple enhancer PLTPpro:Wuschel, Ubipro:Cas9, a ZM U6 Pol111 pro: gRNA cutting at TS45 in ED85E, and a Nospro:CRC. Nospro:CRC inhibits regeneration if stably integrated by producing anthocyanin. Two strains of Agrobacterium tumefaciens, each harboring a binary donor PHP34567 or RV022905 are struck out from a −80° C. frozen aliquot onto separate plates containing 810I medium and incubated for 1-2 days at 28° C. A control vector combination of vector RV022904, which does not contain gRNA cut sites flanking the donor sequence, and RV022905 is used as a control. A single colony from each is picked, struck out onto separate working plates, and allowed to grow overnight at 28° C. The next morning in a sterile hood, the bacteria are individually scraped off and re-suspended in separate conical tubes of 14 ml infection media (700 medium; 5 ml) and 100 mM 3′-5′-Dimethoxy-4′-hydroxyacetophenone (acetosyringone; 5 μL) in a conical tube with a final OD of 0.1-0.2 at a wavelength of 550 nM. The Agrobacterium concentration was approximately 0.5 to 2.0×109 cfu/mL The Agrobacterium LBA4404 suspensions containing PHP34567 and RV022905 are mixed in a 1:1 or 1:9 ratios with final volumes of 1 ml. In both cases, Agrobacterium containing PHP34567 are the equal or larger volume. The suspensions are then used as soon as possible.

Ears of a maize (Zea mays L.) cultivar picked 11 days after pollination are surface sterilized for 15-20 min in 20% (v/v) bleach (5.25% sodium hypochlorite) plus 1 drop of Tween 20 followed by 3 washes in sterile water. Immature embryos are isolated from ears and are placed in 2 ml of the infection medium with acetosyringone solution. Supernatant is removed and 1 ml of the Agrobacterium suspension is added to each tube of embryos. Embryos are transformed with the 1:1 ratio mix for both the control and experimental vector sets. Embryos are transformed with the 1:9 ratio mix for both the control and experimental vector sets. After five minutes, embryos are removed and placed on solid co-cultivation medium plates and oriented embryo axis down. Embryos are incubated at 21° C. in the dark for 1 day. During transformation using the experimental vector sets, Cas9 protein is activated and cuts at three locations: the two gRNA cut sites flanking the HR donor arms, and at the chromosomal/genomic cut site. This frees the donor segment of DNA and allows a higher frequency of HR integration into the genomic cut site.

Embryos are then transferred to resting media and stored at 28C in the dark for 5-7 days. Tissue are then transferred to selection in the dark for 11-14 days.

At 7-12 days after Agrobacterium infection fluorescent multicellular structures are observed which continue to develop and produce plants. In contrast, multicellular structures containing anthocyanin do not regenerate.

Plantlets are moved to rooting with no selection for 7-14 days at 28C in the light. Leafs from individual plants are sampled for qRTPCR to confirm presence or absence of WUS and Cas9, and integration of UBIpro:NPTII at the SDN3 site. Plantlets derived from the negative control Agro mix with no flanking gRNA cut sites are sampled and tested for SDN3 frequency. Some of the negative controls showed integration of UBIpro:NPTII at the cut site. Plantlets derived from the 1:1 Agrobacterium mix of PHP34567 and RV022905 are sampled. Of those, 4 times as many are positive for both HR arms at the genomic cut site, demonstrating homology-dependent recombination (HDR). Of the positive SDN3 samples, all are PCR negative for presence of WUS or Cas9 integration, indicating that the WUS and Cas9 acts transiently without integration. The overall HDR frequency is 4 times greater than the control configuration, which does not have flanking gRNA cut sites.

### Example 10: Co-Transformation Using One a Groba Cterium Containing a Helper Plasmid Plus Two Binary Plasmids, Each with its Own T-DNA. One Vector with a Low Copy Ori has T-DNA Encoding Non-Integrating Cas9 and Wus. The Second Vector with a Higher Copy Ori Contains T-DNA Encoding a Donor Template for Hdr

Co-transformation using one Agrobacterium containing the helper plasmid PHP71539 plus two binary plasmids, each with its own T-DNA. One vector with a low copy origin of replication has T-DNA encoding non-integrating Cas9, gRNA, WUS, and CRC. The second vector with a higher copy origin of replication contains T-DNA encoding a donor template for HDR. PHV00026 (SEQ ID NO. 13) contains a low copy number origin of replication repABC, a nopaline right border, triple enhancer PLTPpro:Wuschel, Ubipro:Cas9, a ZM U6 PolIII pro:gRNA cutting at TS45 in ED85E, and a Nospro:CRC, and an octopine left border. The succinamopine right border in an octopine strain could potentially yield fewer free T-strands created from the Agrobacterium (Sardesai et al. Transgenic Research 27, 539-550 (2018). The vector is in octopine strain LBA4404. A second vector PHV00027 (SEQ ID NO. 14) within the same Agrobacterium contains a pVS1 high copy number origin of replication, an octopine right border, and a donor sequence of HR arms flanking a UBIpro:NPTII, followed by a Ubipro:ZS-GREEN.

This strain ofAgrobacterium harboring PHP71539 plus both PHV00026 and PHV00027 plasmids is struck out from a −80° C. frozen aliquot onto a plate containing 810I medium and incubated for 1-2 days at 28° C. A single colony is picked, struck out onto a working plate, and allowed to grow overnight at 28° C. The next morning in a sterile hood, the bacteria are scraped off and re-suspended in a conical tube of 14 ml infection media (700 medium; 5 ml) and 100 mM 3′-5′-Dimethoxy-4′-hydroxyacetophenone (acetosyringone; 5 μL) with a final OD of 0.1-0.2 at a wavelength of 550 nM. The Agrobacterium LBA4404 suspension containing PHP71539, PHV00026, and PHV00027 are aliquoted to final volume of lml. The suspension is then used as soon as possible.

Ears of a maize (Zea mays L.) cultivar picked 11 days after pollination are surface-sterilized for 15-20 min in 20% (v/v) bleach (5.25% sodium hypochlorite) plus 1 drop of Tween 20 followed by 3 washes in sterile water. Immature embryos are isolated from ears and are placed in 2 ml of the infection medium with acetosyringone solution. Supernatant is removed and 1 ml of the Agro suspension is added to each tube of embryos. A negative control of an Agrobacterium containing similar vectors as outlined above, but not containing cassette for guide RNA is used to transform other embryos. After five minutes, embryos are removed and placed on solid co-cultivation medium plates and oriented embryo axis down. Embryos are incubated at 21° C. in the dark for 1 day. During transformation, two T-strands from the same Agro strain move into the plant cell. The vector with the lower origin of replication copy number and less efficient right border cutting has fewer T-strands produced. This T-strand contains Wus, Cas9, and CRC gene which will produce protein transiently in the transformed plant cell. Cas9 protein is activated and cuts at the chromosomal/genomic cut site, as well as on the gRNA cut sites flanking the HR arms on the second T-Strand, releasing the donor fragment for SDN3.

Embryos are then transferred to resting media and stored at 28° C. in the dark for 5-7 days. Tissue are then transferred to selection in the dark for 11-14 days.

At 7-12 days after Agrobacterium infection, fluorescent multicellular structures are observed which continue to develop and produce plants. In contrast, multicellular structures containing anthocyanin do not regenerate.

Plantlets are moved to rooting with no selection for 7-14 days at 28C in the light. Leaves from individual plants are sampled for qRTPCR to confirm presence or absence of WUS and Cas9, and integration of UBIpro:NPTII at the SDN3 site. 50 Plantlets derived from the negative control agro mix with no gRNA are sampled and tested for SDN3 frequency. None of the negative controls showed integration of UBIpro:NPTII at the cut site. Plantlets derived from the Agrobacterium containing are sampled. Of those, some are PCR positive for both HR arms at the genomic cut site, demonstrating homology-dependent recombination (HDR). Of the positive SDN3 samples, all are PCR negative for presence WUS or Cas9 integration, indicating that the WUS and Cas9 acts transiently without integration.

### Example 11: Sugarcane Inner Whorl Transient Cas9 Activity and Regeneration

A plasmid is used that contains a T-DNA with a 3×ENH::UBI PRO:ZM-WUS2+NOS PRO::CRC+UBI PRO::CAS9, +ZM-U6 PRO, +ALS-MUT1, where the gRNA directs cutting to the endogenous ALS2 gene, the ALS-MUT1 sequence provides a template for editing the endogenous ALS sequence, and the CRC gene inhibits regeneration if stably integrated by producing anthocyanin. The genomic cut site within the ALS gene in conjunction with the ALS-MUT1 editing template provide the components for altering a single base within the endogenous ALS gene, which would confer resistance to the sulfonylurea herbicide chlorsulfuron.

Sugarcane tops are harvested from cultivar CP 88-1762 when six to eight nodes are visible. The outermost leaf sheaths are sterilized 70% ethanol foam and removed under sterile conditions. Immature leaf whorl cross-sections of 2-3 mm thickness are cut from a 0.1 to 5-cm region above the apical meristem. 500 of these segments are co-cultivated with either LBA4404 or AGL1 containing PHPsugar for three days in the dark at 24° C.

Co-cultivated inner whorl segments are incubated for 3 days in the dark at 28° C. on direct embryogenesis medium. During this co-cultivation, T-DNA transiently moves into the nucleus of many cells, and transient expression of gRNA, Cas9, and wuschel occurs. Wuschel gives a boost to embryogenesis while Cas9/gRNA cut at a specific target site within the endogenous ALS gene. If stable integration of the T-DNA occurs, then the cell will not regenerate due to presence of CRCand WUS2. After 7 days, segments are transferred to direct embryogenesis media containing 150 uM chlrosulfuron. After embryogenic shoots start to emerge, the segments are transferred to light at 30 μmol m-2 s-1 with 16 h:8-h (light/dark) photoperiod at 28° C. Somatic embryos are observed between 14-21 days after transformation. Germination of somatic embryos initiate between 30 days after agro co-cultivation. Gene edited shoots, many of which are conferring resistance to chlorsulfuron emerge after 5 weeks and are transferred to rooting media. Shoots are analyzed from both LBA4404 co-cultivated cuttings, and AGL1 co-cultivated cuttings. Of the segments co-cultivated with LBA4404 a number of shoots emerge, and some are positive for edits at the ALS gene. Of these, most or all are negative for presence of the Wuschel, Cas9, and/or guide RNA genes.

### Example 12: Transient Expression for Gene Editing in Dicot Explants

Two Agrobacterium strains are used. The first Agrobacterium strain LBA4404 THY-harboring a T-DNA with a WUS expression cassette, a Cas9 expression cassette, gRNA expression cassette, and a gene expression cassette conferring anti-regeneration if stably expressed, is used. The second Agrobacterium strain contains a gene of interest (trait) and a selectable marker expression cassette. The two Agrobacterium strains are used to co-transform segments of tissue cut from in vitro-grown, sterile, immature soybean explants. Agrobacterium methods, transformation and media progression through co-cultivation and shoot elongation are as previously described.

Soybean lines such as elite lines, including, but not limited to, 93Y21 can be used in this process. Soy tissue explants are harvested and are cut into 3-8 mm sections. An Agrobacterium strain containing the vectors described above are used for the infections, and all bacterial cultures are adjusted to OD550 of 0.5 for infection. All vectors contain the selectable marker gene SPCN (spectinomycin). Explants are infected for 30 min and are placed on co-cultivation medium for three days at 21° C. in dark. After co-cultivation, explants are transferred to shoot regeneration medium. Infection frequency is evaluated by screening the transient expression of the selectable marker gene at 5 and 20 days after transformation. Transient expression of Cas9, gRNA, Wuschel (WUS), and a anti-regeneration gene occur from the first Agrobacterium T-DNA. Transient expression of Cas9 and gRNA create a cut in a specific genomic location/locus. Transient expression of Wuschel can induce cell division. If stable integration of this T-strand occurs, the cell will not divide or regenerate. Integration of a donor DNA carrying a selectable marker from the second T-DNA occurs at the genomic locus cut site. Shoot regeneration is observed about 20 days after infection. In addition, transgenic shoots are evaluated for the presence of the marker gene and absence of Wuschel and Cas9 genes.

Plantlets are moved to rooting with no selection for 1-2 weelks in the light. Leaves from individual plants are sampled for qRTPCR to confirm presence or absence of WUS and Cas9, and integration of selectable marker at the SDN3 site. Of the positive SDN3 samples, all are PCR negative for presence of WUS or Cas9 integration, indicating that the WUS and Cas9 acts transiently without integration. It is believed this editing method will work for other commercially important dicots.

### Example 13: Restoring Function to a Non-Functional Gene Product Via Non-Integrated Guided Cas Systems

As demonstrated in Patent Application US20180282763A1 (incorporated in its entirety here as reference), a disrupted mutant of a chlorsulfuron-resistant ALS allele in maize can be restored through repair of double-stranded breaks generated by Cas9-gRNA cutting in maize. Specifically, the ALS2-P165S gene conferring resistance to chlorsulfuron was further modified (resulting in a disrupted gene): the proline codon encoded by CCG (amino acid 165) in the ALSCas-7 target site was altered by removal of the G nucleotide at the wobble position (3rd nucleotide position in the codon) resulting in the translational frameshift and a disrupted gene (referred to as ALS2-P165S-CCA).

Double-Stranded Breaks (DSB) generated by the Cas9-gRNA system in maize are repaired predominantly through NHEJ often resulting in a single nucleotide insertion at the cleavage site. Due to the fact that proline is encoded by all four of the possible nucleotide insertions at the 3rd wobble position (CCA, CCG, CCT, and CCC) at roughly similar frequencies in maize, any of the four nucleotides being inserted at this position will functionally restore resistance to chlorsulfuron.

As described in patent application U.S. Ser. No. 10/934,536(B2), maize plants (Hi-II genotype) with the specifically-modified the ALS2 gene described above were generated, creating the ALSCas-7 target site that conferred resistance to chlorsulfuron. Then a second round of Cas9/gRNA editing was used to remove the third base in the underlined codon above (removing the “G” from the CCG codon) to produce a frameshift mutant that did not produce a functional protein to confer resistance (this new allele was referred to as ALS2-P165S-CCΔ).

The resultant germplasm containing the disrupted chlorsulfuron-resistant ALS gene (ALS2-P165S-CCΔ) can then be used for retransformation with Cas9 and an appropriate gRNA (ZM-ALS-CR7-1) that result in targeted cutting and insertion of a nucleotide at the cut-site through NHEJ, replacing the missing “G” in the codon and restoring chlorsulfuron resistance. In this manner, Cas9-mediated restoration of this disrupted gene can be used as a selection marker for events which have undergone Cas9-mediated editing. When performed in conjunction with another (unrelated) CAS-mediated genome edit using gRNAs for other genomic sequences/locations, selection using the restored ALS mutant gene enriches the population of TO plants for the second edit.

A. Using One Agrobacterium to Deliver Wus/Cas, and a Second Agrobacterium to Deliver Grna Expression Cassettes for Genome Edits.

A mixture of two Agrobacteria is used the first Agrobacterium containing a T-DNA with expression cassettes for WUS2, ODP2, CAS9, and ZS-GREEN (for example, see PHV00022 in Table 2 for detailed description), and the second Agrobacterium containing a T-DNA with two gRNA expression cassettes that will target cutting in the endogenous maize WAXY locus, plus the gRNA ZM-ALS-CR7-1, and the fluorescent marker DS-RED2 (PHP00024).

Specifically, the first Agrobacterium strain LBA4404 THY-TN- contains both the virulence-promoting helper plasmid (PHP71539; SEQ ID NO. 39) plus the T-DNA-containing plasmid PHV00022 (Table 2 and SEQ ID NO. 15) with the T-DNA containing LOXP +NOS::WUS2+3×ENH::UBI::ODP2+HSP::CRE+LOXP+UBI::CAS9+UBI::ZS-GREEN. The second Agrobacterium contains both PHP71539 plus a T-DNA-containing plasmid PHV00024 (Table 2 and SEQ ID NO. 17) with a T-DNA containing U6::w1 gRNA+U6::w2 gRNA+U6::ALS-CR7 gRNA+UBI::DS-RED2. The two Agrobacterium are grown overnight on solid medium and suspended in infection medium adjusted to the appropriate concentration (OD=0.5-0.6 at 550 nm) as described in EXAMPLE 4. The two Agrobacterium suspensions are then mixed at a 1:1 ratio, and the mixed Agrobacterium suspension is used for seedling-derived leaf transformation of inbred PHH5E, as described in EXAMPLE 4. After the one-week co-cultivation period, the transformed leaf segments are transferred onto chlorsulfuron-containing medium and after 3-4 weeks on selection, healthy somatic embryos are transferred onto maturation medium, then onto rooting medium and finally healthy TO plantlets are transferred to soil in the greenhouse. Using this method, it is expected that a high frequency of TO plants exhibiting resistance to the chlorsulfuron herbicide are recovered, and upon molecular analysis it is determined that a high frequency of the TO plants contain both the restored ALS allele that confers resistance to chlorsulfuron, and dropout of the WAXY gene.

A cut and edit at the ALS gene will confer resistance to the herbicide chlorsulfuron or ethametsulfuron. Although restoration of ALS has been used as an exemplary locus, one could use restoration of function or induction of function of any locus whether it is an endogenous locus or transgenic locus for gene function for positive selection. Agrobacterium methods, transformation and media progression through co-cultivation and shoot elongation are as previously described. Edits at the ALS gene to now confer resistance to chlorsulfuron. Edits to a second trait can also be made, utilizing the other gRNAs.

Plantlets are moved to rooting media for 7-14 days at 28° C. in the light. Leaves from individual plants are sampled for PCR to confirm presence or absence of WUS, ODP2 and Cas9, and base pair editing at the ALS locus. Some plants have an edit conferring resistance at the ALS edited locus and a second trait locus. All are PCR negative for presence WUS, ODP2, or Cas9 integration, indicating that these genes act transiently without integration. Plantlets derived from the negative control agro with no gRNA are sampled and tested editing frequency. None of the negative control plants show editing at the ALS/edited locus.

Based on the results described herein, one skilled in the art can use and expand the described approach to any similarly modified endogenous or pre-integrated exogenous gene(s) replacing co-delivery of a selectable marker gene currently used in plant genome editing experiments. Likewise, this strategy of restoring ALS resistance can be readily adapted to use alternative nucleases to CAS9, such as CAS-alpha10 (see WO2020123887 published 18 Jun. 2020 U.S. Ser. No. 10/934,536(B2)_RTS21920B—US-NP), or such diverse alternative nuclease system such as meganucleases, zinc-finger nucleases, or TALENs.

B. Using a Single Agrobacterium to Deliver a T-DNA Containing Wus/Odp2/Cas9 and Grna for Transient Edits.

An Agrobacterium strain LBA4404 THY-TN- contains both the virulence-promoting helper plasmid (PHP71539; SEQ ID NO. 39) plus the T-DNA-containing plasmid PHV00023 (Table 2 and SEQ ID NO. 16) with the T-DNA containing LOXP+NOS::WUS2+3×ENH::UBI::ODP2+HSP::CRE+LOXP+UBI::CAS9+U6::w1 gRNA+U6::w2 gRNA+U6::ALS-CR7 gRNA+UBI::ZS-GREEN.

The Agrobacterium is grown overnight on solid medium and suspended in infection medium adjusted to the appropriate concentration (OD=0.5-0.6 at 550 nm) as described in EXAMPLE 4, and then used for seedling-derived leaf transformation of inbred PHH5E, as described in EXAMPLE 4. After the one-week co-cultivation period, the transformed leaf segments are transferred onto chlorsulfuron-containing medium and after 3-4 weeks on selection, healthy somatic embryos are transferred onto maturation medium, then onto rooting medium and finally healthy TO plantlets are transferred to soil in the greenhouse. Using this method, it is expected that a high frequency of TO plants exhibiting resistance to the chlorsulfuron herbicide are recovered, and upon molecular analysis it is determined that a high frequency of the TO plants contain both the restored ALS allele that confers resistance to chlorsulfuron, and dropout of the WAXY gene.

A cut and edit at the ALS gene will confer resistance to the herbicide chlorsulfuron or ethametsulfuron. Although restoration of ALS has been used as an exemplary locus, one could use restoration of function or induction of function of any locus whether it is an endogenous locus or transgenic locus for gene function for positive selection. Agrobacterium methods, transformation and media progression through co-cultivation and shoot elongation are as previously described. Edits at the ALS gene now confer resistance to chlorsulfuron. Edits to a second trait can also be made, utilizing the other gRNAs.

Plantlets are moved to rooting media for 7-14 days at 28° C. in the light. Leaves from individual plants are sampled for PCR to confirm presence or absence of WUS, ODP2, and Cas9, and base pair editing at the ALS locus. Some plants have an edit conferring resistance at the ALS edited locus and a second trait locus. All are PCR negative for presence WUS, ODP2, or Cas9 integration, indicating that these genes act transiently without integration. Plantlets derived from the negative control Agrobacterium with no gRNA are sampled and tested editing frequency. None of the negative control plants show editing at the ALS/edited locus.

Based on the results described herein, one skilled in the art can use and expand the described approach to any similarly modified endogenous or pre-integrated exogenous gene(s) replacing co-delivery of a selectable marker gene currently used in plant genome editing experiments. Likewise, this strategy of restoring ALS resistance can be readily adapted to use alternative nucleases to CAS9, such as CAS-alpha10 (Patent Application BB2533PCT (need US Patent No. here, incorporated in its entirety here as reference), or such diverse alternative nuclease system such as meganucleases, zinc-finger nucleases, or TALENs.

### Example 14: Using Agrobacterium to Transform One Cell, but Cas-Alpha or Other Small Cas Endonucleases and Morphogenic Gene Proteins Move to Neighboring Cell(s) to Promote Gene Editing and Regeneration

In another example, any of the methods described herein may benefit from the usage of a smaller endonuclease, such as Cas-alpha (see WO2020123887 published 18 Jun. 2020 U.S. Ser. No. 10/934,536(B2)_RTS21920B—US-NP).

### Example 15: Expressing a Fusion of Dcas to a Cbf1a Activation Domain, Targeted to the R Promoter to Activate Anthocyanin Production

a. CBF1A Activation Domain

Two component transactivation expression systems have been used for many years in plants, for example, see Schwechheiner et al., 1998, Plant Mol. Biol. 36:195-204, in which a fusion protein consisting of the GAL4 DNA-binding domain and the herpes simplex virus PV16 activation domain is expressed behind a promoter such as CaMV 35S, which binds to upstream activation sequences (UAS) in front of a minimal −45 CaMV promoter and the reporter gene beta-glucuronidase. GAL4 UAS is methylated in plants which inhibits binding (Galweiler et al., 2000, Plant J. 23:143-157). As an alternative to GAL4˜VP16, the use of alternative DNA-binding domains such as dCAS9 fused to plant activation domains make a good alternative for two component expression in plants. One such plant transcriptional activation domain comes from the Arabidopsis CBF1A protein (Stockinger et al., 1997, PNAS 94:1035-1040; Wang et al., 2005, Plant Mol. Biol. 58:543-559). For these experiments, the activation domain of the Arabidopsis thaliana CBF1A protein was used (SEQ ID NO. 18).

b. Expression of the control plasmid CRC (or the separate C1 and R genes) produces the phenotype of anthocyanin accumulation in maize cells (Ludwig, S. et al. (1990) Science. 247, 449-450 and Grotewold, E. et al. (2000) Proc Natl Acad Sci USA. 97, 13579-13584).

The fusion protein CRC is composed of the two major domains of the maize C1 transcription factor, the C1-myb domain which binds it's cognate promoter binding sites in target genomic genes (triggering expression of many other genes in the anthocyanin pathway) and the C1 activation domain which stimulates transcription of the targeted genes. These two C1 domains were fused with the maize R gene (a second complementary transcription factor in the anthocyanin pathway) intervening between the two C1 domains, to form the CRC fusion protein (see Bruce et al, 2000, Plant Cell 12(1):65-80). When a construct containing NOS PRO::CRC::SB-GKAF TERM (PHP86489; SEQ ID NO. 20) was introduced into scutellar cells of maize immature embryos using particle bombardment or via Agrobacterium, visible anthocyanin pigment (which accumulates in vacuoles within the expressing cells) was observed starting at 1-4 days after transformation. In a similar manner, when constitutive promoters are used to drive expression of C1 and R in two separate expression cassettes, anthocyanin accumulation is also observed.

c. Expression of C1 alone is insufficient to produce anthocyanin (see Young, J. et al. (2019) Commun Biol. 2, 383).

As a control, particle bombardment is used to introduce the expression cassette CAMV35S ENH::PRO::C1::PINII TERM (PHP665; SEQ ID NO. 21) into scutellar cells of immature embryos, and no anthocyanin is observed. After 1-2 days post-bombardment, and even after weeks of culture, no anthocyanin was observed.

a. As described in Young, J. et al. 2019, Expression of dCAS9:CBF1A+U6 PRO:gRNAr1+U6 PRO:gRNAr2+U6 PRO:gRNAr3+CAMV35S::C1 result in anthocyanin accumulation.

Briefly, plasmids containing a constitutively expressed dCAS9:CBF1A fusion protein (PV225; SEQ ID NO. 22), multiple constitutively expressed gRNA sequences that target the endogenous R promoter (PV521, PV523 and PV528; SEQ ID NO. 23, 24, and 25, respectively), plus a constitutively expressed maize C1 gene (PHP665), were introduced into maize immature embryos. The particle gun was used to introduce UBI PRO::dCAS9:CBF1A::PINII TERM+U6 PRO:gRNAr1+U6 PRO:GRNAr2+U6 PRO:gRNAr3+CAMV35S::C1. One to four days after particle delivery, anthocyanin accumulation was observed in scutellar cells of the infected immature embryos.

In a similar manner, dCASa10 can be fused to repressor motifs or epigenome-modifying factors such as histone deacylase inhibitors (Gilbert et al., 2013, Cell 154(2):442-51), or to CBF300-type proteins (histone acetyltransferase) which locally activate endogenous gene expression (Cheng et al., 2016, Cell Res 26, 254-257).

### Example 16: Expressing a Fusion of Dcas-Alpha10 to a Maize-Optimized Cbf1a Activation Domain, Targeted to the R Promoter to Activate Anthocyanin Production

In this example, the anthocyanin pigmentation pathway in Zea mays cells was activated. Since the coordinated action of two transcription factors, R and C1, are needed to stimulate the production of anthocyanin resulting in a red cellular phenotype (Grotewold et al. (2000) Proceedings of the National Academy of Sciences of the United States of America. 97, 13579-13584), the r gene was targeted for transcriptional upregulation with Cas-alpha while the cl gene was overexpressed from a transgenic construct similar to that described earlier using components of Cas9 or type I-E CRISPR systems (Young et al. (2019) Communications Biology. 2, 383).

A Cas-alpha nuclease expression construct was first engineered to encode a Cas-alpha protein capable of target binding and gene activation. As an example, see FIG. 1 where the A40G+E81G+A87K+T335R (SEQ ID NO: 26) Cas-alpha 10 variant was converted to a nuclease inactive or dead (d) Cas-alpha 10 (SEQ ID NO: 28) and linked with the transcriptional activation domain from the CBF1 protein (SEQ ID NO: 18) using a sequence encoding a flexible connecting peptide (SEQ ID NO: 43). To test the effect of the CBF1 fusion on Cas-alpha 10 dimerization, target recognition and subsequent gene activation, a second Cas-alpha 10 expression construct without the CBF1 domain was also engineered (FIG. 2). Three targets were next selected in the r gene promoter region and sgRNA expression constructs produced. The resulting expression constructs were then co-delivered along with the C1 overexpression cassette into immature corn embryos using particle mediated biolistic transformation as described in EXAMPLE 3. Initially, two experiments were performed. The first experiment used only the CBF1 linked dCas-alpha 10 (FIG. 1) and sgRNA expression cassettes while the second was assembled using a 1:4 mixture of unlinked (FIG. 2) and CBF1 linked dCas-alpha 10 (FIG. 1) expression plasmids along with the sgRNA constructs. After transformation, embryos were exposed to 37° C. for 3 days following transformation as shown in FIG. 3, regimen 2. Four days after transformation, a red anthocyanin phenotype was observed on the surface of embryos from both treatments. Negative control experiments performed without the constructs encoding the r promoter sgRNAs produced no anthocyanin pigmentation indicating that the observed phenotypic change was directly related to the recruitment of CBF1 by dCas-alpha 10 and its sgRNA to the promoter of the r gene. Additionally, experiments performed using only the dCas-alpha10-CBF1 and respective sgRNA expression cassettes resulted in the largest number of anthocyanin positive cells showing that a dimer comprised just of dCas-alpha 10-CBF1 more efficiently activates gene expression than a heterodimer comprised of dCas-alpha 10 and dCas-alpha 10-CBF1 (FIG. 4). Additional Cas-alpha10 variants are disclosed in PCT/US2021/071839, which is incorporated by reference herein, including all of the sequences contained therein.

In a similar manner, dCASa10 can be fused to repressor motifs or epigenome-modifying factors such as histone deacylase inhibitors (Gilbert et al., 2013, Cell 154(2):442-51), or to CBF300-type proteins (histone acetyltransferase) which locally activate endogenous gene expression (Cheng et al., 2016, Cell Res 26, 254-257.

### Example 17. Agrobacterium-Mediated Delivery of a T-DNA Containing Cassettes for High Level Expression of Wus2 and a Diffusible Dcas-Alpha10 Ribonucleoprotein (Rnp) Targeted to Produce Cuts Flanking Waxy, Stimulating Both Somatic Embryo Formation and Waxy Deletions in Neighboring Cells

The Agrobacterium strain LBA4404 THY-TN- (with helper plasmid PHP71539; SEQ ID NO. 39) is used that also contains a plasmid (PHV00015; SEQ ID NO. 42) with a T-DNA containing RB+3×ENH::PLTP PRO::WUS2+NOS::CRC+UBI1ZM PRO::KN1CPP:PROTEIN LINKER1:CAS-ALPHA10 (ALT 35)+U6 PRO:gRNA-w1+U6 PRO:gRNA-w2+LB (where w1 and w2 are gRNA sequences targeting endogenous sequences flanking the maize WAXY gene). In this construct, the gene encoding CAS-ALPHA10 is fused to a flexible linker peptide (PROTEIN LINKER1, SEQ ID NO. 43) and the Cell Penetrating Peptide (CPP) from the maize transcription factor Knotted1 (SEQ ID NO. 44). Other CPP sequences are also known and suitable for use (Numata et al., 2018, Sci Rep 8, 10966; Kardani et al., 2021, J Mol Biol 433:16708). The CPP confers on the fusion protein (KN1CPP:PROTEIN LINKER1:CAS-ALPHA10) the ability to move from cell-to-cell (i.e. from the cell containing the T-DNA expressing the fusion protein into adjacent cells that contain no T-DNA).

A. Diffusible or Intercellular Transportable CAS-ALPHA10 Facilitates Editing in Cells that do not Receive and/or Integrate T-DNA.

Agrobacterium preparation, transformation maize immature embryos, resting, selection, maturation and rooting were done as described in previous EXAMPLES, using the T-DNA-containing plasmid PHV00015. After Agrobacterium infection and co-cultivation, the immature embryos are moved onto resting medium 605B for 7 days, and then all treatments are moved onto selection medium 13266N (13266P plus 150 mg/l G418) for 3 weeks, at which point all somatic embryos are moved through the maturation and rooting steps.

It is expected that i) high levels of WUS2 protein in the cells that receive the T-DNA will stimulate rapid somatic embryo formation in neighboring cells that did not receive the T-DNA, ii) continued high levels of WUS2 and CRC expression in cells that integrate the T-DNA will inhibit regeneration in these cells, and iii) cells that receive the T-DNA will also express KN1CPP:CAS-Alpha10 and the gRNA molecules w1 and w2, which will form KN1CPP:CAS-Alpha10/w1 and KN1CPP:CAS-Alpha10/w2 diffusible RNPs that migrate into neighboring cells and affect CAS-Alpha10-mediated cutting at the w1 and w2 endogenous target sites resulting in deletion of the WAXY gene. In this manner, both stimulation of rapid somatic embryo formation and WAXY deletion occur in neighboring cells to produce edited RO plants.

It should be noted that the duration for the composite culture steps in this protocol are: Agrobacterium infection—30 minutes; co-cultivation—2 days; resting culture—one week; selection culture—3 weeks; maturation—2 weeks; and rooting—2-3 weeks. At this point TO plants are sent to the greenhouse. This timeframe from Agrobacterium infection until the maturation stage is only 4 weeks, 2 days. This demonstrates that Agrobacterium-mediated delivery of WUS2 and KN1CPP:CAS-Alpha10 for targeted genome modification and rapid somatic embryo formation represents a substantially more rapid process than the morphogenic gene-enhanced transformation method reported in the literature by Lowe et al. (2016, Plant Cell 28:1998-2015, incorporated herein for reference) and used for maize CAS9-mediated editing (for example, see (Svitashev et al., 2015, Plant physiology 169:931-945, and Svitachev et al., 2016, Nat Commun 7:13274, incorporated herein for reference).

B. Use of a CAS-ALPHA10 variant allows temperature-controlled editing in cells that do not receive T-DNA

The method described in section “A” above, is used with a plasmid derived from PHV00016 with a T-DNA containing RB+3×ENH::PLTP PRO::WUS2+NOS::CRC+UBI1ZM PRO::KN1CPP:PROTEIN LINKER1:CAS-ALPHA10 (ALT 4)+U6 PRO:gRNA-w1+U6 PRO:gRNA-w2+NOS::CRC+LB, where the CAS-ALPHA10 (ALT4) coding sequence (SEQ ID NO. 54) has been altered to encode a CAS-ALPHA10 (ALT4) protein (SEQ ID NO. 60) which shows no cutting activity at room temperature but high cutting efficiency at 37° C.

When Agrobacterium strain LBA4404 THY-TN- (with helper plasmid PHP71539) containing PHV00016 is used for transformation of PHHSE immature embryos, both the WUS2 and the RNPs produced in the scutellar cell containing the T-DNA move into neighboring scutellar cells, stimulating somatic embryo formation in the neighboring cell. However, cutting at the two genomic target sequences flanking the endogenous WAXY gene (targeted by the gRNAs w1 and w2) does not occur at room temperature, but only after the immature embryos are cultured at 37° C. In this manner, CAS-ALPHA10-mediated editing in cells that have not received (or not integrated) the T-DNA is stimulated under this higher temperature.

C. Delivery of a T-DNA with expression cassettes for WUS2 and two WAXY gRNA sequences into an inbred that contains a pre-integrated UBI1ZM PRO::CAS-ALPHA10 (ALT4).

Stable single-copy transgenic lines of maize inbred PHH5E are produced using Agrobacterium strain LBA4404 THY-TN-(with helper plasmid PHP71539) harboring a plasmid PHV00017 (SEQ ID NO. 55) with a T-DNA containing an cassette for aleurone-specific expression of anthocyanin (Hv-LTP2 PRO::Zm-CRC) plus a constitutively expressed CAS-ALPHA10 (ALT4) gene (RB+Hv-LTP2 PRO::CRC+UBI1ZM PRO::CAS-ALPHA10 (ALT 4)+LB).

Into this temperature-conditional PHH5E inbred containing the pre-integrated temperature-conditional CAS-ALPHA10 (ALT4), a second Agrobacterium strain LBA4404 THY-TN- (with helper plasmid PHP71539) containing the plasmid PHV00018 (SEQ ID NO. 56) is used to deliver a T-DNA containing RB+3×ENH::PLTP PRO::WUS2+U6 PRO:gRNA-w1+U6 PRO:gRNA-w2+Si-UBI PRO::ZS-GREEN+LB. After delivery of this second WUS2/gRNAs/ZS-GREEN T-DNA to a scutellar cell of an immature embryo, both WUS2 protein and the expressed gRNA-w1 and gRNA-w2 diffuse to neighboring cells (not containing the WUS2/gRNAs/ZS-GREEN T-DNA). This initiates rapid somatic embryo formation in the neighboring cell, and when the immature embryo explant is incubated at 37° C., cutting of the w1 and w2 endogenous target sites occurs and the WAXY gene is deleted. After regeneration and crossing of plants containing the WAXY-deleted locus, the pre-existing CAS-ALPHA10 (ALT4) locus is segregated away, to produce progeny plants that contain only with WAXY deletion and no transgene T-DNAs.

D. Inbred maize that containing an integrated single copy of a T-DNA with high temperature conditional CAS-ALPHA10 (ALT4) plus a high temperature-inducible promoter driving expression of WUS2, for later altruistic expression of gRNAs and HDR templates.

Stable single-copy transgenic lines of maize inbred PHH5E are produced using Agrobacterium strain LBA4404 THY-TN-(with helper plasmid PHP71539) harboring a plasmid PHV00019 (SEQ ID NO. 57) with a T-DNA containing RB+HSP17.7 PRO::WUS2+UBI1ZM PRO::CAS-ALPHA10 (ALT 4)+LTP2::CRC+LB, where the HSP17.7 PRO drives no expression (or very attenuated expression) at room temperature, but drives high levels of expression when exposed to 37° C. overnight, or to 45° C. pulses (i.e. for 2-3 hours). This T-DNA is designated as CAS/WUS below, and the maize inbred containing a single copy of the integrated CAS/WUS T-DNA is referred to as PHH5E-CAS/WUS.

Into this single copy inbred line that now exhibits the phenotype of CAS-ALPHA10 (ALT4) cutting and WUS2-stimulated somatic embryo formation upon exposure to high temperature, an Agrobacterium strain LBA4404 THY-TN- (with helper plasmid PHP71539) containing the plasmid PHV00020 (SEQ ID NO. 58) is used to deliver a T-DNA containing RB+U6 PRO::gRNA-53.66+CHR1-53.66 TARGET SITE::SEQ 11-HR1:SI-UBI PRO::NPTII::SI_UBI TERM:SEQ12-HR2::CHR1-53.66 TARGET SITE+SB-UBI PRO::ZS-GREEN:: SB-UBI TERM+LB is used for transformation. This Agrobacterium containing the helper plasmid PHP71539 plus PHV00020 (Agrobacterium 1+Agro1) is adjusted to an OD of 0.5-0.6 at 550 nm and used to transform PHHSE-CAS/WUS immature embryos alone or is mixed with a second Agrobacterium adjusted to the same OD containing only the helper plasmid PHP71539 (Agrobacterium 2+Agro2). The Agro mixture is then used to transform the PHH5E (CAS/WUS) immature embryos. Agro1 can be mixed with Agro2 at a ratio of 100:0, 1000:1, 99:1, 90:10, 75:25, 50:50, 25:75, 10:90, 1:99, 1:1000. At a chosen ratio of Agro1:Agro2 of 10:90, the overall concentration of virulent Agrobacterium cells in the suspension remains high enough for optimal virulence and T-DNA delivery, while the copy number of T-DNAs delivered per cell is reduced, resulting in a high proportion of transformed cells that transiently express the gRNAs and contain the HDR template for targeted integration. In this manner, it is expected that successful HDR with no integration of the PHV00020 template T-DNA occurs at a high frequency.

### Example 18: Expressing Dcas-Alpha10:Cbf1a, Targeted to the Endogenous Wus2 and Bbm Promoter to Stimulate Rapid Somatic Embryo Formation

Agrobacterium strain LBA4404 THY-TN- harboring PHP71539 plus a T-DNA-containing plasmid PHV00021 (SEQ ID NO. 69) was used to transform maize inbred PHH5E immature embryos as described in EXAMPLE 4. This results in the delivery into the plant cells of the T-DNA containing RB+UBI1ZM PRO::dCAS9-ALPHA10:CBF1A::PINII TERM+U6 PRO:gRNA-wus1+U6 PRO:gRNA-wus2+U6 PRO:gRNA-wus3++U6 PRO:gRNA-bbm1+U6 PRO:gRNA-bbm2+U6 PRO:gRNA-bbm3+SB-UBI PRO::ZS-GREEN::OS-UBI TERM+LB.

One to four days after Agrobacterium infection, somatic embryos are formed on the scutellar epithelium of the infected immature embryos, which can rapidly be germinated into fertile plants.

Such a T-DNA can be delivered in a manner that favors transient expression of dCAS9-ALPHA10:CBF1A and the panel of gRNAs, while reducing the likelihood of T-DNA integration. For example, an Agrobacterium containing the helper plasmid PHP71539 plus PHV00021 (Agrobacterium 1+Agro1) is adjusted to an OD of 0.5-0.6 at 550 nm and used to transform PHH5E-CAS/WUS immature embryos alone or is mixed with a second Agrobacterium (both adjusted to the same OD) containing only the helper plasmid PHP71539 (Agrobacterium 2=Agro2). The Agro mixture is then used to transform the PHHSE (CAS/WUS) immature embryos. Agro1 can be mixed with Agro2 at a ratio of 100:0, 1000:1, 99:1, 90:10, 75:25, 50:50, 25:75, 10:90, 1:99, 1:1000. At a preferred ratio of Agro1:Agro2 of 10:90, the overall concentration of virulent Agrobacterium cells in the suspension remains high enough for optimal virulence and T-DNA delivery, while the copy number of T-DNAs delivered per cell is reduced, resulting in a high proportion of transformed cells that transiently express the gRNAs and contain the HDR template for targeted integration. In this manner, it is expected that successful stimulation of somatic embryo formation with no integration of the PHV00021 T-DNA occurs at a high frequency.

We claim:

1. A method of modifying a target site in the genome of a plant cell, comprising:
a. providing to the cell two distinct Rhizobiales bacteria,
i. wherein the first of the two bacteria comprises a first vector, wherein the first vector comprises a heterologous polynucleotide, wherein the heterologous polynucleotide is flanked by polynucleotides comprising homology to a nucleotide sequence at the target site,
ii. wherein the second of the two bacteria comprises a second vector, wherein the second vector comprises a polynucleotide encoding a Cas endonuclease, a guide RNA, at least one morphogenic factor, and an anti-regeneration factor;
wherein the ratio of the amounts of the first vector and the second vector or the ratio of the amount of the two distinct Rhizobiales bacteria is approximately 1:1 to about 10:1, and wherein the Cas endonuclease creates a double-strand break at or near the target site of the plant cell but the polynucleotide encoding the Cas endonuclease does not integrate into the genome of the same plant cell.

2. A method of modifying a target site in the genome of a plant cell, comprising:
a. providing to the cell at least two distinct Rhizobiales bacteria,
i. wherein the first of the two bacteria comprises a first vector, wherein the first vector comprises a gene encoding a guide RNA and a heterologous polynucleotide, wherein the heterologous polynucleotide is flanked by polynucleotides comprising homology to a nucleotide sequence at the target site,
ii. wherein the second of the two bacteria comprises a second vector, wherein the second vector comprises one or more polynucleotides encoding a Cas endonuclease, at least one morphogenic factor, and a gene conferring anti-regeneration properties;
wherein the ratio of the amounts of the first vector and the second vector or the ratio of the amount of the two distinct Rhizobiales bacteria is approximately 1:1 to about 10:1, and wherein the Cas endonuclease creates a double-strand break at or near the target site of the genome of the plant cell but the T-DNA comprising the Cas endonuclease does not integrate into the genome of the same plant cell.

3. A method of modifying a target site in the genome of a plant cell, comprising:
a. providing to the cell one Rhizobiales strain comprising first and second binary plasmids, wherein each of the two binary plasmids comprise distinct T-DNA sequences,
i. wherein the first binary plasmid comprises a low-copy ORI, and a T-DNA encoding a Cas endonuclease and a morphogenic factor,
ii. wherein the second binary plasmid comprises a higher copy ORI and comprises a T-DNA encoding a heterologous polynucleotide,
wherein the ratio of the amounts of the first plasmid and the second plasmid is approximately 1:1 to about 1:10, and wherein the Cas endonuclease creates a double-strand break at or near the target site of the plant cell but does not integrate into the genome of the same plant cell.

4. The method of any one of claims 1-3, wherein the heterologous polynucleotide is integrated through site-directed homologous recombination.

5. The method of any one of claims 1-3, wherein the plant cell is a monocot cell or a dicot cell.

6. The method of any one of claims 1-3, wherein the morphogenic factor is Wuschel.

7. The method of any one of claims 1-3, wherein the Cas endonuclease comprises fewer than about 1500, 1200, 100, 800 and 500 amino acids.

8. The method of any one of claims 1-3, wherein the heterologous polynucleotide is a template for homology-directed repair of a double-strand break at the target site.

9. The method of any one of claims 1-3, wherein at least one of the Rhizobiales is Agrobacterium.

10. The method of any one of claims 1-3, wherein the heterologous polynucleotide is a donor polynucleotide for integration at the target site.

11. A synthetic composition comprising two distinct Rhizobiales bacteria,
a. wherein the first of the two bacteria comprises a first vector, wherein the first vector comprises a heterologous polynucleotide, wherein the heterologous polynucleotide is flanked by polynucleotides comprising homology to a nucleotide sequence at the target site,
b. wherein the second of the two bacteria comprises a second vector, wherein the second vector comprises one or more polynucleotides encoding a Cas endonuclease, a guide RNA, and at least one morphogenic factor;
wherein the ratio of the amounts of the first vector and the second vector is approximately 1:1 to about 10:1.

12. A synthetic composition comprising two different Rhizobiales bacteria,
a. wherein the first of the two bacteria comprises a first vector, wherein the first vector comprises a gene encoding a guide RNA and a heterologous polynucleotide, wherein the heterologous polynucleotide is flanked by polynucleotides comprising homology to a nucleotide sequence at the target site,
b. wherein the second of the two bacteria comprises a second vector, wherein the second vector comprises one or more polynucleotides encoding a Cas endonuclease and at least one morphogenic factor;
wherein the ratio of the amounts of the first vector and the second vector is approximately 1:1 to about 10:1.

13. A synthetic composition comprising one Rhizobiales strain comprising first and second plasmids, wherein each of the two plasmids comprise one or more distinct T-DNA sequences,
a. wherein the first plasmid comprises a low-copy ORI and comprises a T-DNA encoding a Cas endonuclease and a morphogenic factor,
b. wherein the second plasmid comprises a higher copy ORI and comprises a T-DNA encoding a heterologous polynucleotide,
wherein the ratio of the amounts of the first plasmid and the second plasmid is approximately 1:1 to about 1:10.

14. The synthetic composition of any one of claims 11-13 is a plant cell.

15. The synthetic composition of claim 14, wherein the plant cell is a monocot cell or a dicot cell.

16. The synthetic composition of any one of claims 11-13, wherein at least one of the Rhizobiales is Agrobacterium.

17. The synthetic composition of any of claims 11-13, wherein the Cas endonuclease comprises fewer than about 1500, 1200, 100, 800 and 500 amino acids.

18. The synthetic composition of any of claims 11-13 further comprising an anti-regeneration factor.

19. The method of any one of claims 1-3, wherein the plant cell comprising in its genome the polynucleotide encoding the Cas endonuclease is not selected due to the presence or activity of the anti-regeneration factor.

20. The method of any one of claims 1-3, wherein the Cas endonuclease is a Type II or Type V CRISPR-Cas endonuclease.

21. The method of any one of claims 1-3, wherein the plant cell is a haploid plant cell.

22. The method of any one of claims 1-3, wherein the plant cell is a haploid plant cell and the DNA modification happens before or during chromosome doubling stage.

23. The method of any one of claims 1-3, wherein at least one of the Cas endonuclease, the morphogenic factor, and the anti-regeneration factor the plant cell comprises a heterologous cell penetrating peptide (CPP) or an intracellular transfusion domain.

24. A method of modifying a target site in the genome of a plant cell through endogenous activation of one or more genes, the method comprising providing to the cell (a) a first polynucleotide encoding a deactivated CRISPR-Cas polypeptide (dCas) that is capable of site-specifically binding to an endogenous target site comprising a polynucleotide involved in cellular regeneration, but incapable of introducing a double-strand break at the endogenous target site, wherein the dCas is operably linked to a transcriptional activator, the transcriptional activator capable of increasing the expression of the polynucleotide involved in cellular regeneration; (b) providing a second polynucleotide comprising a heterologous polynucleotide flanked by polynucleotides comprising homology to a nucleotide sequence at the target site to be modified; (c) providing a third polynucleotide encoding a Cas endonuclease, a guide RNA, and an anti-regeneration factor;
wherein the ratio of the amounts of the polynucleotide comprising the Cas endonuclease and the polynucleotide comprising the heterologous polynucleotide is approximately 1:1 to about 1:10, and wherein the Cas endonuclease creates a double-strand break at or near the target site of the plant cell but the polynucleotide encoding the Cas endonuclease does not integrate into the genome of the same plant cell.

25. The method of claim 24, wherein the dCas is fused to a transcriptional activation domain capable of initiating transcription from an endogenous morphogenic gene is WUSCHEL, BBM or a combination thereof.

26. The method of claim 24, wherein the polynucleotides are provided through one or more strains of Rhizobiales.

27. The method of claim 24, wherein the polynucleotides are provided through particle bombardment.

28. The method of claim 26, wherein the polynucleotides are present in one or more T-DNAs in the same Agrobacterium strain or two or more distinct Agrobacteria strains.

29. The method of claim 24, wherein the dCas polypeptide is a Type II or a Type V CRISPR-Cas polypeptide.

30. A synthetic composition comprising (a) a first polynucleotide encoding a deactivated CRISPR-Cas polypeptide (dCas) that is capable of site-specifically binding to an endogenous target site comprising a polynucleotide involved in cellular regeneration, but incapable of introducing a double-strand break at the endogenous target site, wherein the dCas is operably linked to a transcriptional activator, the transcriptional activator capable of increasing the expression of the polynucleotide involved in cellular regeneration; (b) providing a second polynucleotide comprising a heterologous polynucleotide flanked by polynucleotides comprising homology to a nucleotide sequence at the target site to be modified; (c) providing a third polynucleotide encoding a Cas endonuclease, a guide RNA, and an anti-regeneration factor.

31. The synthetic composition of claim 30, wherein one or more of the dCas is fused to a cell penetrating peptide.

32. A method of regeneration of a genome modified plant, the method comprising providing one or more polynucleotides that encode a site-specific DNA modifying agent and optionally, a morphogenic factor to a plant cell, wherein the DNA modifying agent and optionally, the morphogenic factor diffuses or is transported into one or more adjacent plant cells; modifying the genome of the one or more adjacent plant cells; regenerating the one or more adjacent plant cells into one or more genome modified plants, wherein the one or more genome modified plants do not comprise the polynucleotide sequence encoding the DNA modifying agent and optionally the morphogenic factor in the absence of a separate segregation step by crossing with another plant.

33. The method of claim 32, wherein the DNA modifying agent is a CRISPR-Cas polypeptide that is capable of moving from one cell to another as a polypeptide or in the form of an RNA sequence capable of being translated into a polypeptide.

34. The method of claim 32, wherein the DNA modifying agent is a CRISPR-Cas polypeptide and is operably linked to a cell penetrating peptide.

35. The method of claim 32, wherein the optional morphogenic factor is operably linked to a cell penetrating peptide.

36. The method of claim 32, wherein the cell that receives the polynucleotide encoding the DNA modifying agent is not selected, but the genome modified cell is regenerated into a genome modified plant.

37. The method of claim 32, wherein the DNA modifying agent is a CRISPR-Cas polypeptide and the guide RNA is encoded by the same polynucleotide that encodes the CRISPR-Cas polypeptide.

38. The method of claim 32, wherein the optional morphogenic factor is encoded by an expression construct that is distinct from the polynucleotide that encodes the DNA modifying agent.

39. The method of claim 32, wherein the polynucleotide that encodes the DNA modifying agent, a guide RNA and the optional morphogenic factor are present in one or more distinct expression cassettes.

40. The method of claim 32, wherein the DNA modifying agent is a CRISPR-Cas deactivated polypeptide (dCas).

41. The method of claim 32, further comprising a heterologous donor polynucleotide.

42. The method of claim 32, wherein the polynucleotide that encodes the DNA modifying agent and the optional morphogenic factor are present in the plant cell at a ratio of about 1:1 to about 1:5, 1:8, 1:10 and to about 1:100.

43. The method of claim 32, wherein the DNA modifying agent is a base editor.
